SEQUENCE LISTING (37 sequences)

SEQ ID NO: 1 - DNA, 2399 bp, Mus musculus
cccgcccacccgccctttatattccgggggtctgcgcggccgaggacccctgggctgcgctgctctcagctgccgggtccgactcgcctcactcagctcccctcctgcctcctgaagggcagggcttcgccgacgcttggcgggaaaaagaagggaggggagggatcctgagtcgcagtataaaagaagcttttcgggcgtttttttctgactcgctgtagtaattccagcgagagacagagggagtgagcggacggttggaagagccgtgtgtgcagagccgcgctccggggcgacctaagaaggcagctctggagtgagaggggctttgcctccgagcctgccgcccactctccccaaccctgcgactgacccaacatcagcggccgcaaccctcgccgccgctgggaaactttgcccattgcagcgggcagacacttctcactggaacttacaatctgcgagccaggacaggactccccaggctccggggagggaatttttgtctatttggggacagtgttctctgcctctgcccgcgatcagctctcctgaaaagagctcctcgagctgtttgaaggctggatttcctttgggcgttggaaaccccgcagacagccacgacgatgcccctcaacgtgaacttcaccaacaggaactatgacctcgactacgactccgtacagccctatttcatctgcgacgaggaagagaatttctatcaccagcaacagcagagcgagctgcagccgcccgcgcccagtgaggatatctggaagaaattcgagctgcttcccaccccgcccctgtccccgagccgccgctccgggctctgctctccatcctatgttgcggtcgctacgtccttctccccaagggaagacgatgacggcggcggtggcaacttctccaccgccgatcagctggagatgatgaccgagttacttggaggagacatggtgaaccagagcttcatctgcgatcctgacgacgagaccttcatcaagaacatcatcatccaggactgtatgtggagcggtttctcagccgctgccaagctggtctcggagaagctggcctcctaccaggctgcgcgcaaagacagcaccagcctgagccccgcccgcgggcacagcgtctgctccacctccagcctgtacctgcaggacctcaccgccgccgcgtccgagtgcattgacccctcagtggtctttccctacccgctcaacgacagcagctcgcccaaatcctgtacctcgtccgattccacggccttctctccttcctcggactcgctgctgtcctccgagtcctccccacgggccagccctgagcccctagtgctgcatgaggagacaccgcccaccaccagcagcgactctgaagaagagcaagaagatgaggaagaaattgatgtggtgtctgtggagaagaggcaaacccctgccaagaggtcggagtcgggctcatctccatcccgaggccacagcaaacctccgcacagcccactggtcctcaagaggtgccacgtctccactcaccagcacaactacgccgcacccccctccacaaggaaggactatccagctgccaagagggccaagttggacagtggcagggtcctgaagcagatcagcaacaaccgcaagtgctccagccccaggtcctcagacacggaggaaaacgacaagaggcggacacacaacgtcttggaacgtcagaggaggaacgagctgaagcgcagcttttttgccctgcgtgaccagatccctgaattggaaaacaacgaaaaggcccccaaggtagtgatcctcaaaaaagccaccgcctacatcctgtccattcaagcagacgagcacaagctcacctctgaaaaggacttattgaggaaacgacgagaacagttgaaacacaaactcgaacagcttcgaaactctggtgcataaactgacctaactcgaggaggagctggaatctctcgtgagagtaaggagaacggttccttctgacagaactgatgcgctggaattaaaatgcatgctcaaagcctaacctcacaaccttggctggggctttgggactgtaagcttcagccataattttaactgcctcaaacttaaatagtataaaagaacttttttttatgcttcccatcttttttctttttccttttaacagatttgtatttaattgtttttttaaaaaaatcttaaaatctatccaattttcccatgtaaatagggccttgaaatgtaaataactttaataaaacgtttataacagttacaaaagattttaagacatgtaccataattttttttatttaaagacattttcatttttaaagttgatttttttctattgtttttagaaaaaaataaaataattggaaaaaatac

SEQ ID NO: 2 - protein, 454 aa, Mus musculus
MDFLWALETPQTATTMPLNVNFTNRNYDLDYDSVQPYFICDEEENFYHQQQQSELQPPAPSEDIWKKFELLPTPPLSPSRRSGLCSPSYVAVATSFSPREDDDGGGGNFSTADQLEMMTELLGGDMVNQSFICDPDDETFIKNIIIQDCMWSGFSAAAKLVSEKLASYQAARKDSTSLSPARGHSVCSTSSLYLQDLTAAASECIDPSVVFPYPLNDSSSPKSCTSSDSTAFSPSSDSLLSSESSPRASPEPLVLHEETPPTTSSDSEEEQEDEEEIDVVSVEKRQTPAKRSESGSSPSRGHSKPPHSPLVLKRCHVSTHQHNYAAPPSTRKDYPAAKRAKLDSGRVLKQISNNRKCSSPRSSDTEENDKRRTHNVLERQRRNELKRSFFALRDQIPELENNEKAPKVVILKKATAYILSIQADEHKLTSEKDLLRKRREQLKHKLEQLRNSGA

SEQ ID NO: 3 - DNA, 2377 bp, Homo sapiens
acccccgagctgtgctgctcgcggccgccaccgccgggccccggccgtccctggctcccctcctgcctcgagaagggcagggcttctcagaggcttggcgggaaaaagaacggagggagggatcgcgctgagtataaaagccggttttcggggctttatctaactcgctgtagtaattccagcgagaggcagagggagcgagcgggcggccggctagggtggaagagccgggcgagcagagctgcgctgcgggcgtcctgggaagggagatccggagcgaatagggggcttcgcctctggcccagccctcccgctgatcccccagccagcggtccgcaacccttgccgcatccacgaaactttgcccatagcagcgggcgggcactttgcactggaacttacaacacccgagcaaggacgcgactctcccgacgcggggaggctattctgcccatttggggacacttccccgccgctgccaggacccgcttctctgaaaggctctccttgcagctgcttagacgctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaaggaaaagtaaggaaaacgattccttctaacagaaatgtcctgagcaatcacctatgaacttgtttcaaatgcatgatcaaatgcaacctcacaaccttggctgagtcttgagactgaaagatttagccataatgtaaactgcctcaaattggactttgggcataaaagaacttttttatgcttaccatcttttttttttctttaacagatttgtatttaagaattgtttttaaaaaattttaagatttacacaatgtttctctgtaaatattgccattaaatgtaaataactttaataaaacgtttatagcagttacacagaatttcaatcctagtatatagtacctagtattataggtactataaaccctaattttttttatttaagtacattttgctttttaaagttgatttttttctattgtttttagaaaaaataaaataactggcaaatatatcattgagccaaaaaaaaaaaaaaaaaaaaa

SEQ ID NO: 4 - protein, 454 aa, Homo sapiens
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 5 - DNA, 5148 bp, Mus musculus
  CDS (217)..(2106)
ccacttccctagttacagacaccacttaacagcaggaaacctccacttgctctcctctgccttctctccattgcagccattcccattttcaagactgccagtctggtcctttcaacgttccatgctgcctggtgcccgggagcccaggccttctgtgactccaggacaggaaggtacttctccatacacttcccagaactctgaaggggtgcagacatggcgggaagtgctgtggacagcgccaaccacctgacctacttttttggcaacatcacccgggaagaggctgaagactacctggtccagggaggcatgaccgatgggctctacctgctacgccagagccgcaattacctgggtggttttgctttgtcggtggctcacaacaggaaggcacaccactacaccatcgagagggaacttaatggcacctacgccatctccgggggcagggcccatgccagcccagcagacctctgccattaccactcccaggaacctgatggccttatctgcctccttaagaagcccttcaaccggcccccgggagtacagcccaagaccggaccctttgaggacctgaaggagaacctcatcagggaatatgtgaaacagacctggaaccttcagggccaggctctggagcaagccatcatcagccagaagccccagctggagaagctgatcgccaccacggcccatgagaagatgccctggttccatggcaacatctccagagatgaatcagagcagacggtcctcatagggtcaaagaccaatggaaaattcctgatcagggccagagacaacagcggctcctatgctctgtgcctgctgcacgaagggaaagtattgcactaccgcattgacagggacaagaccgggaagctctccattcctgaggggaagaagtttgacaccctctggcagctagtggaacattactcttacaagccagatgggctactaagagtcctcacggtaccatgccaaaagattggtgcacagatgggccacccaggaagcccaaatgcccatcccgtgacttggtcaccgggtggaataatctcaaggatcaaatcctactccttcccaaagcctggccacaaaaagcctgccccaccccaagggagccgtccagagagcactgtgtccttcaacccctatgagccaacgggagggccctggggcccagacagaggccttcagagagaagccctgcccatggacacagaggtgtacgagagcccttatgctgaccctgaagagatccggcccaaagaggtctacctggacaggagcctgctgaccctggaggacaatgaactgggctccggtaacttcgggactgtgaaaaagggatactaccaaatgaaaaaagttgtgaaaaccgtggctgtgaaaatcctgaagaacgaggccaacgacccggctttgaaggacgagctgctggcagaggcgaacgtcatgcagcagctggacaacccctacattgtgcgcatgatcggaatctgcgaggcggagtcctggatgctggtgatggagatggcggagctggggccgctcaacaagtacctgcagcagaacaggcacattaaggataagaacatcatagagctggttcaccaggtttccatggggatgaagtatttggaagagagcaactttgtgcacagagatctggctgcgcggaacgtgcttctggtcacacagcactatgccaagatcagcgatttcggtctttccaaagccctgcgtgctgatgaaaactactacaaggcccagacccacgggaagtggcccgtgaagtggtacgcccccgaatgcatcaactactacaagttctccagtaagagtgacgtctggagcttcggagtcctgatgtgggaagcgttctcctatgggcagaagccctacagagggatgaaagggagcgaagtgaccgccatgctggagaaaggagagcggatggggtgccctgcaggatgcccgagagagatgtacgacctgatgaacctgtgctggacttacgatgtggagaacaggccaggattcacagctgtggaactgaggcttcgcaattactactacgacgtggttaactaacagctccggcgcctgtccgtgcacaccacggattcccaagcgatcacaggaaattcattcagatgaactggctctcagagtttcatctccctctgcccggagtgagagctaagctaacataggactcaccctcacaacaggtctggtgcccagagacagacaagcagcaagaccttggggcctgtggagtcgtctctctggtctttgtttccatctgtgtggtcttcgctatcggtcacgtttgggagccatttccaaattctcttgacatcattccgttcctctgggtccaggatttcagtgtcccctgaagatcagaaaagaaaagtgctttgccactccagcaaaggacataaagagaaacctcgctggtgcagtcagcaagctggaaggtggttaaggacaatgacaagcctggacactggttttactctcagcgctgagacagagatactgtgtggatgacatgctgagatgtgctggcttggctgacacgcccctggagtccacctccctgcagtcgtgtgtgtttgctggaggaggtgtgaataacgttctgtgtggaggctgccgggggaggtgagctcccatgatcctctgcgactgagcagaagtgtgtcaggcaagggtcagactcccctctctgcagagggaatgcatccaggactccttgttgctgccactaaaaaattgccaaccagcaacttaagggaggaatggctcatttgagctcacagttggaggggataggccatcatggtggaaaagacaacaggcaggaaaggcttggtggcatgaaaaggaagctggaaggtcacatggcatccacattcaggaaacaaagaatgaacaggaagtggggccaggctataaggccctaaaggccacctccggtgacccagttccttcagatccatctatcaaagattccacaggcctctcagacactgaatgccaacagctggggaccaaatgttcaagcacacaaggtgcagtgccaggcaggggccttcctgaatgattacacagtgtggtatcctgacagagcgggtgagccatctcaggtctgtccccttatgagccccgtttaccatcccagattcccccttgggacccccctcatctggatcattcccctccccccaggccctatctccaaacacctaggtactaagtgaacctaggtactaagtcttcaacacatgaattctgaggggacacattcatactggagccgatggcagcctggtgaccaagaccatctcaggatgagtttctgaatggaatcttcattcgccataaaaccagagatgtgaccttccccacaaggaagccttgctaagtgcgacattgagctgaggacacacaagctgagaattccccctctgtcctcctagccttggctaactactgaccatacaccgaatgtcacccattcacggcacttttgctgaggacctaaaagctacaaaggctgcatagccttccatctaggtctagggacatgtacagatgggagcatctcaccttcacctctgagacactgggacattcacagccagggatggtacaggagtccctgctggtggtgggctcaatgaacagagacgatggtgaaatgggtgcctctctttagaccagtggttctcaaccttcctaatgctgagatctttcaatacaggtccttgcgttgtggcgactcccaaccatgaagttattttgttgctacattgtcactgtaattttgctactatgatgaatcgtagtgtagatatctgatatgcaggatatcggatatgtaaatatctggtatgcaggatatgtgaaatgagggcacatcccacaggttgggaaccactgcctttgatagggaggcactatgcttagagcatggttaggaagtatttagaagctattggtgtgtcttggaattgtgttagtaggtcaaccgtcctgtggagggaaggaagggggtggaggaaatccctgtatgttgaattcattgagcagacctgcagaacacagccttggtgaggaagtcccatgggtcagaccacctgtagatttagtcaggagaggaagggccactgtatagttatggagacaggactgtctgctctgagcaagaagttctctgctgagctcctagcctaccttcccttgtccccagggctatagaaaggccacctcgaagaccagggagcatgtgtgagaagtctgtggaggcctctgccttcttcctggcctctctagcagtgcctctccttcccagggatttgggactgaccgttatctggctatgactgagcagagtggagggatgggctttgccccccttcagaaagccgaaaccggttcccatgtgcagagtcacatgtggccatcgaccagggctctcatctggtgaaaactgtgtcttactgtcaagattaattctctcccctgggccactgaggagggcagatggcaagaggatcctccctagaggatttagactatgaatgcccactaaatttgcaaggtcagaaactagccaaggtccttctcaggcatctatccttaacttggtctctccacaagctgcctctgatgctccgagccccttctgcctggcagctgggctccctgtgtgcttttacctatgacccttggtcagcagagtacaaggagcacctaagctgcctcgccacctcccactgactgcaaacaccacatacctcatgcggaaccgaatgtctctccagtaaacaccccagccagccattcataaaaacctgtctctgtgtgcgctgggacatgtcctccctgtaccccgggcctgctttgtgtgccagacagtgactccacagggatgccaggtctgtgattgcattgctccttgcagagggaagccctcggttgtccttctctgggaatgctgccacccagagcagcacacagtgattccctgttatttcagaaattattttaaataaagatctactattagtcttgtgaaaaaaaaaaaaaaaaaaaaaaaaaaaaaaaaaaaaaaaaaaaaaa

SEQ ID NO: 6 - protein, 629 aa, Mus musculus
MAGSAVDSANHLTYFFGNITREEAEDYLVQGGMTDGLYLLRQSRNYLGGFALSVAHNRKAHHYTIERELNGTYAISGGRAHASPADLCHYHSQEPDGLICLLKKPFNRPPGVQPKTGPFEDLKENLIREYVKQTWNLQGQALEQAIISQKPQLEKLIATTAHEKMPWFHGNISRDESEQTVLIGSKTNGKFLIRARDNSGSYALCLLHEGKVLHYRIDRDKTGKLSIPEGKKFDTLWQLVEHYSYKPDGLLRVLTVPCQKIGAQMGHPGSPNAHPVTWSPGGIISRIKSYSFPKPGHKKPAPPQGSRPESTVSFNPYEPTGGPWGPDRGLQREALPMDTEVYESPYADPEEIRPKEVYLDRSLLTLEDNELGSGNFGTVKKGYYQMKKVVKTVAVKILKNEANDPALKDELLAEANVMQQLDNPYIVRMIGICEAESWMLVMEMAELGPLNKYLQQNRHIKDKNIIELVHQVSMGMKYLEESNFVHRDLAARNVLLVTQHYAKISDFGLSKALRADENYYKAQTHGKWPVKWYAPECINYYKFSSKSDVWSFGVLMWEAFSYGQKPYRGMKGSEVTAMLEKGERMGCPAGCPREMYDLMNLCWTYDVENRPGFTAVELRLRNYYYDVVN

SEQ ID NO: 7 - DNA, 2672 bp, Homo sapiens
  CDS (148)..(2055)
aggaagagccgcgggcccggcggctgaggccaccccggcggcggctggagagcgaggaggagcgggtggccccgcgctgcgcccgccctcgcctcacctggcgcaggtggacacctgcgcaggtgtgtgccctccggcccctgaagcatggccagcagcggcatggctgacagcgccaaccacctgcccttctttttcggcaacatcacccgggaggaggcagaagattacctggtccaggggggcatgagtgatgggctttatttgctgcgccagagccgcaactacctgggtggcttcgccctgtccgtggcccacgggaggaaggcacaccactacaccatcgagcgggagctgaatggcacctacgccatcgccggtggcaggacccatgccagccccgccgacctctgccactaccactcccaggagtctgatggcctggtctgcctcctcaagaagcccttcaaccggccccaaggggtgcagcccaagactgggccctttgaggatttgaaggaaaacctcatcagggaatatgtgaagcagacatggaacctgcagggtcaggctctggagcaggccatcatcagtcagaagcctcagctggagaagctgatcgctaccacagcccatgaaaaaatgccttggttccatggaaaaatctctcgggaagaatctgagcaaattgtcctgataggatcaaagacaaatggaaagttcctgatccgagccagagacaacaacggctcctacgccctgtgcctgctgcacgaagggaaggtgctgcactatcgcatcgacaaagacaagacagggaagctctccatccccgagggaaagaagttcgacacgctctggcagctagtcgagcattattcttataaagcagatggtttgttaagagttcttactgtcccatgtcaaaaaatcggcacacagggaaatgttaattttggaggccgtccacaacttccaggttcccatcctgcgacttggtcagcgggtggaataatctcaagaatcaaatcatactccttcccaaagcctggccacagaaagtcctcccctgcccaagggaaccggcaagagagtactgtgtcattcaatccgtatgagccagaacttgcaccctgggctgcagacaaaggcccccagagagaagccctacccatggacacagaggtgtacgagagcccctacgcggaccctgaggagatcaggcccaaggaggtttacctggaccgaaagctgctgacgctggaagacaaagaactgggctctggtaattttggaactgtgaaaaagggctactaccaaatgaaaaaagttgtgaaaaccgtggctgtgaaaatactgaaaaacgaggccaatgaccccgctcttaaagatgagttattagcagaagcaaatgtcatgcagcagctggacaacccgtacatcgtgcgcatgatcgggatatgcgaggccgagtcctggatgctagttatggagatggcagaacttggtcccctcaataagtatttgcagcagaacagacatgtcaaggataagaacatcatagaactggttcatcaggtttccatgggcatgaagtacttggaggagagcaattttgtgcacagagatctggctgcaagaaatgtgttgctagttacccaacattatgccaagatcagtgatttcggactctccaaagcactgcgtgctgatgaaaactactacaaggcccagacccatggaaagtggcctgtcaagtggtacgctccggaatgcatcaactactacaagttctccagcaaaagcgatgtctggagctttggagtgttgatgtgggaagcattctcctatgggcagaagccatatcgagggatgaaaggaagtgaagtcaccgctatgttagagaaaggagagcggatggggtgccctgcagggtgtccaagagagatgtacgatctcatgaatctgtgctggacatacgatgtggaaaacaggcccggattcgcagcagtggaactgcggctgcgcaattactactatgacgtggtgaactaaccgctcccgcacctgtcggtggctgcctttgatcacaggagcaatcacaggaaaatgtatccagaggaattgattgtcagccacctccctctgccagtcgggagagccaggcttggatggaacatgcccacaacttgtcacccaaagcctgtcccaggactcaccctccacaaagcaaaggcagtcccgggagaaaagacggatggcaggatccaaggggctagctggatttgtttgttttcttgtctgtgtgattttcatacaggttatttttacgatctgtttccaaatccctttcatgtctttccacttctctgggtcccggggtgcatttgttactcatcgggcccagggacattgcagagtggcctagagcactctcaccccaagcggccttttccaaatgcccaaggatgccttagcatgtgactcctgaagggaaggcaaaggcagaggaatttggctgcttctacggccatgagactgatccctggccactgaaaagctttcctgacaataaaaatgttttgaggctttaaaaagaaaatcaagtttgaccagtgcagtttctaagcatgtagccagttaaggaaagaaagaaaaaaaaaaaaaaaaaa

SEQ ID NO: 8 - protein, 635 aa, Homo sapiens
MASSGMADSANHLPFFFGNITREEAEDYLVQGGMSDGLYLLRQSRNYLGGFALSVAHGRKAHHYTIERELNGTYAIAGGRTHASPADLCHYHSQESDGLVCLLKKPFNRPQGVQPKTGPFEDLKENLIREYVKQTWNLQGQALEQAIISQKPQLEKLIATTAHEKMPWFHGKISREESEQIVLIGSKTNGKFLIRARDNNGSYALCLLHEGKVLHYRIDKDKTGKLSIPEGKKFDTLWQLVEHYSYKADGLLRVLTVPCQKIGTQGNVNFGGRPQLPGSHPATWSAGGIISRIKSYSFPKPGHRKSSPAQGNRQESTVSFNPYEPELAPWAADKGPQREALPMDTEVYESPYADPEEIRPKEVYLDRKLLTLEDKELGSGNFGTVKKGYYQMKKVVKTVAVKILKNEANDPALKDELLAEANVMQQLDNPYIVRMIGICEAESWMLVMEMAELGPLNKYLQQNRHVKDKNIIELVHQVSMGMKYLEESNFVHRDLAARNVLLVTQHYAKISDFGLSKALRADENYYKAQTHGKWPVKWYAPECINYYKFSSKSDVWSFGVLMWEAFSYGQKPYRGMKGSEVTAMLEKGERMGCPAGCPREMYDLMNLCWTYDVENRPGFAAVELRLRNYYYDVVN

SEQ ID NO: 9 - DNA, 60 bp, Mus musculus
tactctgcacactgggacggtgattactgcggccaacggtctctggtctctgtctctgca

SEQ ID NO: 10 - DNA, 60 bp, Mus musculus
tattgtgcaaactgggaccgtgattaccgcggccaagggactctggtccctgtctctgca

SEQ ID NO: 11 - DNA, 60 bp, Mus musculus
tactgtgcaaactaagacggtgattactgcggctaagggactctggtcactgtctctgca

SEQ ID NO: 12 - DNA, 60 bp, Mus musculus
tactgtgcaaactgggacggtgattactgcagtcaagggcctctggtctctgtctctgca

SEQ ID NO: 13 - DNA, 60 bp, Mus musculus
tcctatgcaaactgggacggtgattactgcggccaagggactctggtcactgtctctgca

SEQ ID NO: 14 - DNA, 60 bp, Mus musculus
tactgtgcaaactgggacggtgattactgcggccaagggactctgatcattgtctcggca

SEQ ID NO: 15 - DNA, 60 bp, Mus musculus
tactgtgcaaactgggacgatgattcctgcggccttgggactctggtcgctgtctctgca

SEQ ID NO: 16 - DNA, 60 bp, Mus musculus
tactgtgcaaactgggacggtgattactgctgccacgggtccctggtccctgtctcttca

SEQ ID NO: 17 - DNA, 60 bp, Mus musculus
tactgtgcaaactagaacggtgattactgcggccaagggaccctggtcactgtctctgca

SEQ ID NO: 18 - DNA, 60 bp, Mus musculus
tactgtgcaaactgggacggtgattactacgtccaagggactctggtcactgtctctgca

SEQ ID NO: 19 - DNA, 60 bp, Mus musculus
tactgtgcaaactgggacggtgattactgcggccaagggactctggtcactgtctctgca

SEQ ID NO: 20 - DNA, 19 bp, Homo sapiens
gtcgagcattattcttata

SEQ ID NO: 21 - DNA, 19 bp, Homo sapiens
ggaataatctcaagaatca

SEQ ID NO: 22 - DNA, 19 bp, Homo sapiens
aagctttcctgacaataaa

SEQ ID NO: 23 - DNA, 19 bp, Mus musculus
ggcagctagtggaacatta

SEQ ID NO: 24 - DNA, 19 bp, Mus musculus
gcaactttgtgcacagaga

SEQ ID NO: 25 - DNA, 19 bp, Mus musculus
gtggaacattactcttaca

SEQ ID NO: 26 - DNA, 55 bp, Homo sapiens
tgtcgagcattattcttatattcaagagatataagaataatgctcgacttttttc

SEQ ID NO: 27 - DNA, 55 bp, Homo sapiens
tggaataatctcaagaatcattcaagagatgattcttgagattattccttttttc

SEQ ID NO: 28 - DNA, 55 bp, Homo sapiens
taagctttcctgacaataaattcaagagatttattgtcaggaaagcttttttttc

SEQ ID NO: 29 - DNA, 55 bp, Mus musculus
tggcagctagtggaacattattcaagagataatgttccactagctgccttttttc

SEQ ID NO: 30 - DNA, 55 bp, Mus musculus
tgcaactttgtgcacagagattcaagagatctctgtgcacaaagttgcttttttc

SEQ ID NO: 31 - DNA, 55 bp, Mus musculus
tgtggaacattactcttacattcaagagatgtaagagtaatgttccacttttttc

SEQ ID NO: 32 - DNA, 59 bp, Homo sapiens
tcgagaaaaaagtcgagcattattcttatatctcttgaatataagaataatgctcgaca

SEQ ID NO: 33 - DNA, 59 bp, Homo sapiens
tcgagaaaaaaggaataatctcaagaatcatctcttgaatgattcttgagattattcca

SEQ ID NO: 34 - DNA, 59 bp, Homo sapiens
tcgagaaaaaaaagctttcctgacaataaatctcttgaatttattgtcaggaaagctta

SEQ ID NO: 35 - DNA, 59 bp, Mus musculus
tcgagaaaaaaggcagctagtggaacattatctcttgaataatgttccactagctgcca

SEQ ID NO: 36 - DNA, 59 bp, Mus musculus
tcgagaaaaaagcaactttgtgcacagagatctcttgaatctctgtgcacaaagttgca

SEQ ID NO: 37 - DNA, 59 bp, Mus musculus
tcgagaaaaaagtggaacattactcttacatctcttgaatgtaagagtaatgttccaca

(19) United States
(12) Patent Application Publication
(10) Pub. No.: US 2008/0070256 A1
(43) Pub. Date: Mar. 20, 2008

(54) Non-Human Animal Models for B-cell Non-Hodgkin's Lymphoma and Uses Thereof

(75) Inventors: Yosef Refaeli, Denver, CO (US); Brian Curtis Turner, Denver, CO (US); Ryan Young, Denver, CO (US)

Correspondence Address: SHERIDAN ROSS PC, 1560 BROADWAY, SUITE 1200, DENVER, CO, 80202, US

(73) Assignee: NATIONAL JEWISH MEDICAL AND RESEARCH CENTER, Denver, CO (US)

(21) Appl. No.: 11/829,058

(22) Filed: Jul. 26, 2007

Related U.S. Application Data
(60) Provisional application No. 60/820,478, filed on Jul. 26, 2006.

(51) Int. Cl. A01K 67/00; C12N 5/00; C12N 5/02; C12Q 1/68

(52) U.S. Cl. 435/6; 435/355; 435/375; 800/10

(57) ABSTRACT

Disclosed are non-human animal models, and preferably, rodent models, and more preferably, mouse models, of B-cell Non-Hodgkin's Lymphoma (NHL). In particular, the present invention provides animal models of B cell NHL including, B cell chronic lymphocytic leukemia/lymphoma (B-CLL), Burkitt's lymphoma (BL), Follicular-like lymphoma (FLL) and Diffuse large B-cell lymphoma (DLBCL), as well as various methods for producing these non-human animal models. These animal models, as well as cell lines produced from or derived from these models, are useful tools for a variety of methods, including, but not limited to, preclinical testing of drug candidates, and particularly drug candidates that are specific for human proteins, and any research, development, pharmaceutical, or clinical purpose, including but not limited to, the identification, development, and/or testing of drugs (therapeutics, prophylactics, etc.), targets, markers, and/or research tools for use in the diagnosis of, study of, or treatment of any Non-Hodgkin's Lymphoma, such as those described herein, or for any related condition.

### CROSS-REFERENCE TO RELATED APPLICATIONS

This application claims the benefit of priority under 35 U.S.C. §119(e) from U.S. Provisional Application No. 60/820,478, filed Jul. 26, 2006. The entire disclosure of U.S. Provisional Application No. 60/820,478 is incorporated herein by reference for all purposes.

### FIELD OF THE INVENTION

This invention generally relates to the provision of several different non-human animal models for various forms of B-cell non-Hodgkin's Lymphomas (NHL), as well as cells, cell lines and tumors derived from these models. The invention also relates to methods of use of such animal models, as well as cell lines produced or derived from these animal models, for the preclinical testing of drug candidates, including those specific for human proteins, as well as a variety of research, development, pharmaceutical, or clinical purposes, including the identification of novel molecular targets important in B cell NHL.

### BACKGROUND OF THE INVENTION

The notion of a role for chronic inflammation in lymphomagenesis has been popular for many years. There have been indications that antigenic stimulus can play a role in lymphomagenesis. First, retroviral infection of mice elicits T-cell lymphomas only in those strains of mice that can mount an immune response to the virus (McGrath, M. S., and Weissman, I. L. (1979) Cell 17, 65-75; Lee, J. C., and Ihle, J. N. (1981) Nature 289, 407-9). Second, infection with Helicobacter pylori is an apparent cause of human lymphomas in mucosal associated lymphoid tissue (MALT) and gut associated lymphoid tissue (GALT) (Jones, R. G., Trowbridge, D. B., and Go, M. F. (2001). Front. Biosci. 6, E213-26). Treatment with antibiotics to eradicate infection elicits remission of these tumors, as if they might have been sustained by antigenic stimulus from the microbe (Casella et al. (2001) Anticancer Res 21, 1499-502; Montalban et al., (2001). Gut 49, 584-7). Third, mice with graft versus host disease consequent to bone marrow transplantation frequently develop T-cell lymphomas; immunosuppression of the mice prevents the tumors (Gleichmann et al. (1971). Verh. Dtsch. Ges. Inn. Med. 77, 1153-4). Fourth, chronic antigenic stimulation by infection may contribute to the genesis of Burkitt's lymphoma (BL) (Klein, U., Klein, G., Ehlin-Henriksson, B., Rajewsky, K., Kuppers, R. (1995). Burkitt's lymphoma is a malignancy of mature B-cells expressing somatically mutated V region genes. Mol Med 1, 495-505; Chapman, C. J., Wright, D., Sevenson, F. K. (1998) Insight into Burkitt's lymphoma from immunoglobulin variable region gene analysis. Leuk lymphoma 30, 257-67). Fifth, the gene expression profiles of diffuse large B-cell lymphomas resemble those of B-cells that have mounted a response to antigen (Alizadeh et al., (2000). Nature 403, 503-11), and the tumor cells display high affinity antigen receptors on their surface, as if they had been subjected to the selective pressure of an antigen (Kuppers, R., Rajewski, K., and Hansmann, M. L. (1997). Diffuse large cell lymphomas are derived from mature B cells carrying V region genes with a high load of somatic mutation and evidence of selection for antibody expression. Eur J Immunol 27, 1398-1405; Ottesmeier, C. H., Thompsett, A. R., Zhu, D., Wilkins, B. S., Sweetenham, J. W., and Stevenson, F. K. (1998). Analysis of Vh genes in follicular and diffuse lymphoma shows ongoing somatic mutation and multiple isotype transcripts in early disease with changes during disease progression. Blood 91, 4292-4299; Lossos, I. S., Alizabeth, A. A., Eisen, M. B., Chan, W. C., Brown, P. O., Botstein, D., Staudt, L. M., and Levy, R. (2000). Ongoing immunoglobulin somatic mutation in germinal center B cell-like but not in activated B cell-like diffuse large cell lymphomas. Proc. Natl. Acad. Sci. USA 97, 10209-13; Lossos, I. S., Okada, C. Y., Warnke, J. M., Greiner, T. C., and Levy, R. (2000). Molecular analysis of immunoglobulin genes in diffuse large B-cell lymphomas. Blood 95, 1797-1803). These findings prompt the hypothesis that an antigenic stimulus may cooperate with other tumorigenic influences in the genesis of lymphoma.

A number of genetic lesions have been implicated in the genesis of lymphoid tumors. One such genetic alteration involves the dysregulation of the proto-oncogene MYC. The MYC gene encodes a short-lived, transcriptionally active protein that is expressed in many tissues. MYC is highly regulated in lymphoid tissues. This gene was originally identified as the cellular version of the viral oncogene, v-myc. The proto-oncogene MYC plays an important role in the control of cellular proliferation, size, differentiation and apoptosis. The molecular mechanism by which MYC regulates those cellular processes remains unclear; however, it probably involves some form of transcriptional activity. Overexpression of MYC has been implicated in diverse forms of human tumors. The overexpression can result from a variety of mechanisms, including chromosomal translocation and gene amplification. Lymphomas figure prominently among the tumors in which MYC has been incriminated. This may reflect important roles played by MYC in the regulation of lymphoid-cell development, proliferation and survival.

The number of newly diagnosed cases of non-Hodgkin's lymphoma (NHL) has increased by almost 80% in the last 25 years. This dramatic increase in newly diagnosed cases does not correlate with age, gender or infectious agents, and cannot be accounted for by the onset of HIV-associated B-cell lymphomas. As a result, NHL currently account as the fifth most common form of cancer in the United States, after breast, prostate, lung and colon cancer. NHL is one of the few cancers whose incidence and mortality rates have risen in the past 35 years. Despite the increase in the incidence of NHL, the etiology of these lymphomas remains elusive, and current therapeutic approaches rely on traditional, non-specific chemotherapeutic approaches. Accordingly, there is a need in the art for improved therapies and therapeutic approaches for the treatment of NHL, as well as for a better understanding of the mechanisms by which the various forms of cancer encompassed by NHL are initiated and progress. To this end, animal models of NHL, including the individual forms of NHL, that closely mimic the human disease, would be exceedingly valuable.

### SUMMARY OF THE INVENTION

One embodiment of the invention relates to a non-human animal model of B cell chronic lymphocytic leukemia/lymphoma (B-CLL), wherein the animal model is a transgenic non-human animal that expresses the following transgenes: (a) a MYC transgene that is overexpressed in the B cell lineage in the animal; and (b) a transgene encoding a pre-rearranged B cell receptor (BCR) that is overexpressed in the B cell lineage of the animal.

Another embodiment of the invention relates to a non-human animal model of B cell chronic lymphocytic leukemia/lymphoma (B-CLL), wherein the animal model is a transgenic non-human animal that expresses the following transgenes: (a) a MYC transgene that is overexpressed in the B cell lineage in the animal; (b) a transgene encoding a non-rearranged Ig heavy chain of a B cell receptor (BCR) that selectively binds to an antigen; (c) a transgene encoding a lambda light chain of a BCR that binds to the antigen of (b).

Yet another embodiment of the invention relates to a non-human animal model of Burkitt's Lymphoma (BL), wherein the animal model is a transgenic non-human animal that expresses the following transgenes: (a) a MYC transgene, wherein the MYC transgene comprises a nucleic acid sequence encoding MYC coupled to an inducible expression control sequence, wherein B cell-specific expression of the MYC transgene can be selectively repressed; (b) a transgene encoding a pre-rearranged B cell receptor (BCR) that is overexpressed in the B cell lineage of the animal and that selectively binds to an antigen; and (c) a transgene encoding a soluble form of the antigen bound by the BCR in (ii), wherein the transgene is expressed so that it is available systemically in the animal. The B cell-specific expression of the MYC transgene is not repressed in the animal.

Another embodiment of the invention relates to a non-human animal model of Burkitt's Lymphoma (BL), wherein the animal model is a transgenic non-human animal that expresses the following transgenes: (a) a MYC transgene that is overexpressed in the B cell lineage in the animal; (b) a pre-rearranged VDJ region of an Ig heavy chain of a B cell receptor (BCR) that selectively binds to an antigen, wherein the transgene is designed to be integrated into the Ig heavy chain locus of the animal; (c) a transgene encoding a lambda light chain of a BCR that binds to the antigen of (ii); and (d) a transgene encoding a soluble form of the antigen in (ii) and (iii), wherein the transgene is expressed so that it is available systemically in the animal.

Yet another embodiment of the present invention relates to a non-human animal model of Burkitt's Lymphoma (BL), wherein the animal model is a transgenic non-human animal that expresses the following transgenes: (a) a MYC transgene that is overexpressed in the B cell lineage in the animal; and (b) a transgene encoding a pre-rearranged B cell receptor (BCR) that is overexpressed in the B cell lineage of the animal and that binds to an endogenous self-antigen expressed by the animal.

Another embodiment of the present invention relates to a non-human animal model of Follicular Like Lymphoma (FLL), comprising a transgenic non-human animal that expresses: (a) a MYC transgene, wherein the MYC transgene comprises a nucleic acid sequence encoding MYC coupled to an inducible expression control sequence, wherein B cell-specific expression of the MYC transgene can be selectively repressed; (b) a transgene encoding a pre-rearranged B cell receptor (BCR) that is overexpressed in the B cell lineage of the animal and that selectively binds to an antigen; and (c) a transgene encoding a soluble form of the antigen bound by the BCR in (b), wherein the transgene is expressed so that it is available systemically in the animal. The B cell-specific expression of the MYC transgene was repressed in the animal from the birth of the animal until the animal was an adult, followed by a lowered level of continued repression of the expression of the MYC transgene, to induce FLL in the animal.

Yet another embodiment of the invention relates to a non-human animal model of Follicular Like Lymphoma (FLL), comprising a transgenic non-human animal that expresses: (a) a MYC transgene, wherein the MYC transgene comprises a nucleic acid sequence encoding MYC coupled to an inducible expression control sequence, wherein B cell-specific expression of the MYC transgene can be selectively repressed; (b) a transgene encoding a pre-rearranged B cell receptor (BCR) that is overexpressed in the B cell lineage of the animal and that binds to an endogenous self-antigen expressed by the animal. The B cell-specific expression of the MYC transgene was repressed in the animal from the birth of the animal until the animal was an adult, followed by a lowered level of continued repression of the expression of the MYC transgene, to induce FLL in the animal.

Another embodiment of the invention relates to a non-human animal model of Diffuse Large B Cell Lymphoma (DLBCL), comprising a transgenic non-human animal that expresses: (a) a MYC transgene, wherein the MYC transgene comprises a nucleic acid sequence encoding MYC coupled to an inducible expression control sequence, wherein B cell-specific expression of the MYC transgene can be selectively repressed; (b) a transgene encoding a pre-rearranged B cell receptor (BCR) that is overexpressed in the B cell lineage of the animal and that selectively binds to an antigen; and (c) a transgene encoding a soluble form of the antigen bound by the BCR in (b), wherein the transgene is expressed so that it is available systemically in the animal. The B cell-specific expression of the MYC transgene in the animal was repressed from the birth of the animal until the animal was an adult, followed by cessation of the repression of the expression of the MYC transgene in the animal, to induce DLBCL in the animal.

Yet another embodiment of the invention relates to a non-human animal model of Diffuse Large B Cell Lymphoma (DLBCL), comprising a transgenic non-human animal that expresses: (a) a MYC transgene, wherein the MYC transgene comprises a nucleic acid sequence encoding MYC coupled to an inducible expression control sequence, wherein B cell-specific expression of the MYC transgene can be selectively repressed; (b) a transgene encoding a pre-rearranged B cell receptor (BCR) that is overexpressed in the B cell lineage of the animal and that binds to an endogenous self-antigen expressed by the animal. The B cell-specific expression of the MYC transgene in the animal was repressed from the birth of the animal until the animal was an adult, followed by cessation of the repression of the expression of the MYC transgene in the animal, to induce DLBCL in the animal.

In any of the above-described embodiments of the invention, in one aspect, the MYC transgene comprises a nucleic acid sequence encoding MYC coupled to an Ig heavy chain enhancer. In one aspect, the MYC transgene is Eμ-MYC.

In any of the above-described embodiments of the invention, in one aspect, the MYC transgene can comprise a nucleic acid sequence encoding MYC coupled to an inducible expression control sequence, wherein B cell-specific expression of the MYC transgene can be selectively repressed. In one aspect, such a MYC transgene comprises a nucleic acid sequence encoding Myc coupled to a tetracycline (TET) responsive expression control element (TRE), the expression of which is controlled by a B-cell specific TET-off repressor. In one aspect, the B-cell specific TET-off repressor is a mouse mammary tumor virus long term repeat (MMTV-LTR) driving expression of reverse tetracycline-dependent transactivator (rtTA) (MMTV-rtTA). In one aspect, the MYC transgene is TRE-MYC and wherein the animal also expresses an MMTV-rtTA transgene. In one aspect, the expression of the MYC transgene can be selectively repressed by administration of tetracycline or doxycycline.

In any of the above-described embodiments of the invention, the BCR transgene can be expressed under the control of the endogenous immunoglobulin promoter. In one aspect, the BCR transgene selectively binds to hen egg lysozyme (HEL).

In any of the above-described embodiments of the invention, the BCR transgene can comprise: (a) a pre-rearranged VDJ region of an Ig heavy chain of a B cell receptor (BCR) that selectively binds to an antigen, wherein the transgene is designed to be integrated into the Ig heavy chain locus of the animal; and (b) a transgene encoding a lambda light chain of a BCR that binds to the antigen of (b). In one aspect, the BCR transgene selectively binds to hen egg lysozyme (HEL).

In any of the above-described embodiments of the invention, if the BCR binds to a self-antigen, the can BCR binds to arsenate and to an endogenous self-antigen in the animal. Such a BCR transgene includes, but is not limited to, ARS.A1.

In any of the above-described embodiments of the invention, in one aspect, the transgene encoding the soluble form of the antigen is sHEL.

In any of the above-described embodiments of the invention, in one aspect, the animal is a rodent, including, but not limited to, a mouse.

One embodiment of the invention relates to a non-human animal model of B-CLL that is a transgenic mouse that expresses the following transgenes: (a) Eμ-MYC; and (b) BCL^{HEL}.

Another embodiment of the invention relates to a non-human animal model of B-CLL that is a transgenic mouse that expresses the following transgenes: (a) TRE-MYC; (b) MMTV-rtTA; and (c) BCL^{HEL}.

Yet another embodiment of the invention relates to a non-human animal model of B-CLL that is a transgenic mouse that expresses the following transgenes: (a) Eμ-MYC; (b) VDJki; and Lt-tg.

Another embodiment of the invention relates to a non-human animal model of B-CLL that is a transgenic mouse that expresses the following transgenes: (a) TRE-MYC; (b) MMTV-rtTA; (c) VDJki; and (d) Lt-tg.

Another embodiment of the invention relates to a non-human animal model of BL that is a transgenic mouse that expresses the following transgenes: (a) TRE-MYC; (b) MMTV-rtTA; (c) BCL^{HEL}; and (d) sHEL. B cell-specific expression of the TRE-MYC transgene is not repressed in the animal.

Yet another embodiment of the invention relates to a non-human animal model of BL that is a transgenic mouse that expresses the following transgenes: (a) Eμ-MYC; (b) VDJki; (c) Lt-tg; and (d) sHEL.

Another embodiment of the invention relates to a non-human animal model of BL that is a transgenic mouse that expresses the following transgenes: (a) Eμ-MYC; and (b) Ars.A1.

Another embodiment of the invention relates to a non-human animal model of FLL that is a transgenic mouse that expresses the following transgenes: (a) TRE-MYC; (b) MMTV-rtTA; (c) BCL^{HEL}; and (d) sHEL. B cell-specific expression of the TRE-MYC transgene was repressed in the animal by administration of tetracycline or doxycycline from the birth of the animal until the animal was an adult, followed by continued administration of a lower amount of tetracycline or doxycycline to the animal, to induce FLL in the animal.

Yet another embodiment of the invention relates to a non-human animal model of FLL that is a transgenic mouse that expresses the following transgenes: (a) TRE-MYC; (b) MMTV-rtTA; and (c) ARS.A1. B cell-specific expression of the TRE-MYC transgene was repressed in the animal by administration of tetracycline or doxycycline from the birth of the animal until the animal was an adult, followed by continued administration of a lower amount of tetracycline or doxycycline to the animal, to induce FLL in the animal.

Another embodiment of the invention relates to a non-human animal model of DLBCL that is a transgenic mouse that expresses the following transgenes: (a) TRE-MYC; (b) MMTV-rtTA; (c) BCL^{HEL}; and (d) sHEL. B cell-specific expression of the TRE-MYC transgene in the animal was repressed by administration of tetracycline or doxycycline from the birth of the animal until the animal was an adult, followed by cessation of the administration of tetracycline or doxycycline to the animal, to induce DLBCL in the animal.

Yet another embodiment of the invention relates to a non-human animal model of DLBCL that is a transgenic mouse that expresses the following transgenes: (a) TRE-MYC; (b) MMTV-rtTA; and (c) ARS.A1. B cell-specific expression of the TRE-MYC transgene in the animal was repressed by administration of tetracycline or doxycycline from the birth of the animal until the animal was an adult, followed by cessation of the administration of tetracycline or doxycycline to the animal, to induce DLBCL in the animal.

Another embodiment of the invention relates to a B cell isolated from any of the non-human animal models described herein.

Yet another embodiment of the invention relates to a B cell line produced from a B cell isolated from any of the non-human animal models described herein.

Another embodiment of the invention relates to B cell tumors isolated from any of the non-human animal models described herein.

Yet another embodiment of the invention relates to a panel of transgenic mice for evaluation of non-Hodgkin's lymphomas (NHL), comprising two or more different transgenic mouse models selected from any of the models as described herein and/or a transgenic mouse model of Burkitt's Lymphoma (BL), wherein the transgenic mouse expresses: (a) a MYC transgene that is overexpressed in the B cell lineage in the animal; (b) a transgene encoding a pre-rearranged B cell receptor (BCR) that is overexpressed in the B cell lineage of the animal; and (c) a transgene encoding a soluble form of the antigen bound by the BCR in (b), wherein the transgene is expressed so that it is available systemically in the animal. In one aspect, the transgenic mouse expresses the following transgenes: (i) Eμ-MYC; (ii) BCR^{HEL}; and (iii) sHEL.

Another embodiment of the invention relates to a panel of two or more B cell lines, comprising at least one B cell line was produced from B cells isolated from any transgenic mouse as described herein.

Yet another embodiment of the invention relates to a method for preclinical testing of drug candidates for non-Hodgkin's Lymphoma (NHL), comprising administering to any one or more of the non-human animal models described herein a candidate drug for NHL, and detecting whether the candidate drug inhibits tumors in the animal model, wherein candidate drugs that inhibit tumors in the animal model are selected for clinical testing.

Another embodiment of the invention relates to a method for preclinical testing of drug candidates for non-Hodgkin's Lymphoma (NHL), comprising administering to two or more mouse models in any of the panels of mice described herein, a candidate drug for NHL, and detecting whether the candidate drug inhibits tumors in the one or more of the mouse models in the panel of mice, wherein candidate drugs that inhibit tumors in at least one mouse model is selected for clinical testing. In one aspect, a candidate drug that inhibits tumors in a first mouse model but not in a second mouse model is selected for clinical testing as a specific inhibitor of the form of NHL exhibited by the first mouse model.

In any of the above-described methods, in one aspect, the mouse model has been further genetically modified to comprise a human nucleic acid molecule of interest or to express a human protein, wherein the nucleic acid molecule or protein is a target for human NHL, and wherein the method includes a step of detecting whether the candidate drug changes the expression or biological activity of the target as compared to in the absence of the candidate drug. In one aspect, the target is expressed by the tumors of the mouse model.

Yet another embodiment of the invention relates to a method for preclinical testing of drug candidates for non-Hodgkin's Lymphoma (NHL), comprising contacting any one or more of the B cell lines described herein with a candidate drug for NHL, and detecting whether the B cell line is sensitive to the candidate drug, wherein candidate drugs to which the B cell line is sensitive is selected for clinical testing.

Another embodiment of the invention relates to a method for preclinical testing of drug candidates for non-Hodgkin's Lymphoma (NHL), comprising administering to two or more of the B cell lines in any of the panels of B cell lines described herein, a candidate drug for NHL, and detecting whether one or more of the B cell lines is sensitive to the candidate drug, wherein candidate drugs to which at least one B cell line is sensitive is selected for clinical testing. In one aspect, a candidate drug to which a B cell line from a first mouse model is sensitive, but to which a B cell line from a second mouse model is not sensitive, is selected for clinical testing as a specific inhibitor of the form of NHL exhibited by the first mouse model.

In one aspect of any of the above-described methods, the B cell line has been genetically modified to comprise a human nucleic acid molecule of interest or to express a human protein, wherein the nucleic acid molecule or protein is a target for human NHL, and wherein the method includes a step of detecting whether the candidate drug changes the expression or biological activity of the target as compared to in the absence of the candidate drug.

Yet another embodiment of the invention relates to a method to identify a target for use in the diagnosis, study or treatment of a Non-Hodgkin's Lymphoma (NHL) or condition related thereto, comprising comparing the expression of genes by any one or more of the animal models described herein to the expression of genes by a control animal that does not have an NHL, and identifying genes that are differentially expressed in the NHL animal model, or the proteins encoded by the genes, as targets for use in the diagnosis, study or treatment of NHL.

Another embodiment of the invention relates to a method to identify a target for use in the diagnosis, study or treatment of a Non-Hodgkin's Lymphoma (NHL) or condition related thereto, comprising comparing the expression of genes by two or more of any of the mouse models in any panel of mice described herein to each other and to the expression of the genes by a control mouse that does not have an NHL, and identifying genes that are differentially expressed in one or more of the NHL mice models, or the proteins encoded by the genes, as targets for use in the diagnosis, study or treatment of NHL. In one aspect, a gene that is differentially expressed in a first mouse model but not in a second mouse model is selected as a specific target for the form of NHL exhibited by the first mouse model.

Yet another embodiment of the invention relates to a method to identify a target for use in the diagnosis, study or treatment of a Non-Hodgkin's Lymphoma (NHL) or condition related thereto, comprising comparing the expression of genes by any B cell line described herein to the expression of genes by a control B cell line from an animal that does not have NHL, and identifying genes that are differentially expressed in the B cell line from the NHL animal model, or the proteins encoded by the genes, as targets for use in the diagnosis, study or treatment of NHL.

Another embodiment of the invention relates to a method to identify a target for use in the diagnosis, study or treatment of a Non-Hodgkin's Lymphoma (NHL) or condition related thereto, comprising comparing the expression of genes by two or more B cell lines from any of the panels of B cell lines described herein to each other and to the expression of genes by a control B cell line from an animal that does not have NHL, and identifying genes that are differentially expressed in one or more of the B cell line from the NHL mouse models, or the proteins encoded by the genes, as targets for use in the diagnosis, study or treatment of NHL. In one aspect, a gene that is differentially expressed in B cell line from a first mouse model but not in a B cell line from a second mouse model is selected as a specific target for the form of NHL exhibited by the first mouse model.

Another embodiment of the invention relates to a method to identify a target for use in the diagnosis, study or treatment of a Non-Hodgkin's Lymphoma (NHL) or condition related thereto, comprising comparing a biological activity of a gene or protein any of the animal models described herein to the biological activity of the gene or protein in a control animal, wherein genes or proteins with a difference in biological activity in the animal model as compared to the control animal are selected for use in the diagnosis, study or treatment of NHL.

Yet another embodiment of the invention relates to a method to identify a target for use in the diagnosis, study or treatment of a Non-Hodgkin's Lymphoma (NHL) or condition related thereto, comprising comparing a biological activity of a gene or protein two or mice in any of the panels of mice described herein to each other and to the biological activity of the gene or protein by a control mouse that does not have an NHL, wherein genes or proteins with a difference in biological activity in one or more of the mouse models as compared to the control animal, are selected for use in the diagnosis, study or treatment of NHL. In one aspect, genes or proteins having a difference in biological activity in a first mouse model but not in a second mouse model is selected as a specific target for the form of NHL exhibited by the first mouse model.

Another embodiment of the invention relates to a method to identify a target for use in the diagnosis, study or treatment of a Non-Hodgkin's Lymphoma (NHL) or condition related thereto, comprising comparing a biological activity of a gene or protein in any B cell line described herein to the biological activity of the gene or protein in a control B cell line from an animal that does not have NHL, wherein genes or proteins with a change in biological activity in the B cell line from the animal model as compared to the B cell line from the control animal, are selected for use in the diagnosis, study or treatment of NHL.

Yet another embodiment of the invention relates to a method to identify a target for use in the diagnosis, study or treatment of a Non-Hodgkin's Lymphoma (NHL) or condition related thereto, comprising comparing a biological activity of a gene or protein in two or more B cell lines from any panel of B cell lines described herein to each other and to the biological activity of the gene or protein in a control B cell line from an animal that does not have NHL, wherein genes or proteins with a change in biological activity in a B cell line from the animal model as compared to the B cell line from the control animal, are selected for use in the diagnosis, study or treatment of NHL. In one aspect, a gene or protein having a difference in biological activity in a B cell line from a first mouse model but not in a B cell line from a second mouse model is selected as a specific target for the form of NHL exhibited by the first mouse model.

In one aspect of any of the above-described methods, the method comprises a first step of contacting the animal or cells with a test compound, prior to the step of comparing.

Another embodiment of the invention relates to a method to inhibit a B cell Non-Hodgkin's Lymphoma (NHL), comprising inhibiting tec tyrosine kinase Syk expression or activity. In one aspect, the NHL is selected from the group consisting of: B-cell chronic lymphocytic leukemia, Burkitt's lymphoma, Follicular like lymphoma and Diffuse large B-cell lymphoma. In one aspect, the method comprises administering to an NHL an shRNA that selectively binds to Syk and inhibits the expression of Syk. In one aspect, the method comprises administering to an NHL a drug that inhibits the expression or activity of Syk.

Yet another embodiment of the invention relates to a non-human animal model of Non-Hodgkin's Lymphoma (NHL), wherein the animal model is a transgenic non-human animal that overexpresses MYC in an autoreactive B cell background.

Another embodiment of the invention relates to a non-human animal model of Non-Hodgkin's Lymphoma (NHL), wherein the animal model is a transgenic non-human animal that overexpresses MYC in a background where the B cell receptor is tonically or constitutively expressed.

In either of the above embodiments, in one aspect, the non-human animal model is a mouse.

Another embodiment of the invention relates to an isolated non-human animal egg, wherein the egg contains the transgenes expressed by any one of the non-human animal models described herein.

Yet another embodiment of the invention relates to a part of any of the non-human animal models described herein, selected from the group consisting of: a cell, a tissue, an organ or a bodily fluid.

Another embodiment of the invention relates to the use of any of the non-human animal models described herein for preclinical testing of drug candidates.

Yet another embodiment of the invention relates to the use of any of the non-human animal models described herein to identify, develop, and/or test a compound for use in the diagnosis of, study of, or treatment of any Non-Hodgkin's Lymphoma or condition related thereto.

Another embodiment of the invention relates to the use of any of the non-human animal models described herein to identify, develop, and/or test a target for use in the diagnosis of, study of, or treatment of any Non-Hodgkin's Lymphoma or condition related thereto.

Another embodiment of the invention relates to the use of an inhibitor of tec tyrosine kinase Syk in the preparation of a medicament for the prophilaxis and treatment of B-cell Non-Hodgkin's Lymphomas, including B-cell chronic lymphocytic leukemia, Burkitt's lymphomas, Follicular like lymphomas and Diffuse large B-cell lymphomas.

### BRIEF DESCRIPTION OF THE DRAWINGS OF THE INVENTION

FIG. 1 is a graph showing that the clonal B-cell antigen receptor cooperates with MYC in the development of B cell lymphomas.

FIGS. 2A-2D are graphs showing lymphomagenesis in the mouse model for B-CLL. Single cell suspensions were generated from lymph nodes (FIG. 2A; six nodes for each mouse—a pair of inguinal, axillary and brachial lymph nodes), spleens (FIG. 2B), thymii (FIG. 2C) and jaw-tumors (FIG. 2D). Open bars represent normal mice. Filled bars represent tumor-bearing mice. The graphs represent the total number of cells (×10-6) obtained for the indicated organs. Counts represent the mean derived from 10 independent mice±the standard deviation for those values.

FIGS. 3A-3F are digital images showing the histological analysis of tumors in Eμ-MYC/BCR^{HEL} mice. Tissues were sectioned, stained with hematoxylin and eosin, and microscopic images (FIGS. 3A and 3D show spleen from a normal wild type mouse; FIGS. 3B and 3E show lymph node tumor from an Eμ-MYC mouse; FIGS. 3C and 3F show spleen tumor from an Eμ-MYC/BCR^{HEL} mouse).

FIGS. 4A-4I are graphs showing the suppression of tumor growth by pharmacological agents. The recipient mice were held until tumors became clinically apparent. Tumor recipient (clear bars) and wild type (filled bars) mice then received daily injections of the indicated drugs for 7 days of either cyclosporine A (csa), FK506, rapamycin (rap) or cyclophosphamide (cyph). For FIGS. 4A-4H, the mice were euthanized 24 hours after the last injection of drug, and lymph nodes were harvested for analysis of either total number of cells (FIGS. 4A-4D) or surface markers of donor cells (FIGS. 4E-4I). For FIG. 4I, the mice were observed over a span of 100 days and deaths recorded, as shown. (FIGS. 4A and 4E show Eμ-MYC tumors; FIGS. 4B and 4F show Eμ-MYC/BCR^{HEL} tumors; FIGS. 4C and 4G show Eμ-MYC/BCR^{HEL}/sHEL tumors; FIGS. 4D and 4H show MMTV-rtTA/TRE-MYC/BCR^{HEL}/sHEL tumors; FIG. 4I shows the survival of animals bearing Eμ-MYC/BCR^{HEL}/sHEL tumors.

FIGS. 5A-5F are digital images showing the histological analysis of tumors in the mouse models of BL (FIGS. 5A and 5D show spleen from a normal wild type mouse; FIGS. 5B and 5E show spleen tumor from an Eμ-MYC/BCR^{HEL}/sHEL mouse; FIGS. 5C and 5F show jaw tumor from an MMTV-rtTA/TRE-MYC/BCR^{HEL}/sHEL mouse.

FIGS. 6A-6F are graphs showing the establishment and maintenance of murine BL by antigenic stimulus and MYC overexpression (FIGS. 6A and 6B show primary transplants; FIGS. 6C and 6D show secondary transplants; FIGS. 6E and 6F show B-cell lymphomas regress after MYC overexpression is extinguished; FIGS. 6A and 6C show cohorts of mice on regular food; FIGS. 6B and 6D show cohorts of mice on doxycycline-containing food; FIG. 6E shows a cohort of mice on regular food until 16 days after development of externally visible lymphadenopathy, followed by a doxycycline-containing diet; FIG. 6F shows a cohort of mice on regular food until 40 days after spontaneous tumor development, followed by a doxycycline containing diet.)

FIGS. 7A-7H are digital images showing the clonality of tumors in the mouse models of the invention (FIG. 7A shows wild type spleen; FIG. 7B shows spleen cells from a 6 month old MRLlpr/lpr mouse with lymphoproliferative disease; FIG. 7C shows spleen tumor from an Eμ-MYC/BCR^{HEL} mouse; FIG. 7D shows spleen cells from a mouse 60 days after receiving a transplant of the cells analyzed in FIG. 7C; FIG. 7E shows spleen tumor from an Eμ-MYC/BCR^{HEL}/sHEL mouse; FIG. 7F shows spleen cells from a mouse 23 days after receiving a transplant of the cells analyzed in FIG. 7E; FIG. 7G shows a jaw tumor from an MMTV-rtTA/TRE-MYC/BCR^{HEL}/sHEL mouse; FIG. 7H shows spleen cells from a mouse 14 days after receiving a transplant of cells analyzed in FIG. 7G.

FIG. 8 is a series of flow cytometry images showing the detection of tumor cells in FLL and DLBCL transgenic mice of the invention using flow cytometry. Lymphoid organs obtained from different mice were used to generate single cell suspensions. Those cells were stained for surface expression of either B220 (Y-axis), or Thy1.1 and Mac-1 (X-Axis), and analyzed by flow cytometry.

FIG. 9 is a series of digital images showing the histological analysis of tumors in FLL, DLBCL, and BL mouse models of the invention. Spleens were sectioned, stained with hematoxylin and eosin, and microscopic images obtained. Magnification was 10× for top panels, 25× for middle panels and 100× for bottom panels.

FIG. 10 is a series of digital images showing the histological analysis of tumors in FLL and DLBCL mice of the invention. Spleens were sectioned, stained with hematoxylin and eosin, and microscopic images obtained. Magnification was 100× for all panels.

FIG. 11 shows the differential expression of chemokine receptors on large B-cell lymphomas established in mice. Single cell suspensions were generated from the spleens obtained from tumor bearing mice (WT, wild-type; Q-BL, Burkitt's like tumor with jaw tumor, FLL, MMTV-tTA/TRE-MYC/BCR^{HEL}/sHEL mice that were transferred to a diet containing a low concentration of Doxycycline at 4 months of age; or DLBCL, MMTV-tTA/TRE-MYC/BCR^{HEL}/sHEL mice that were transferred to a diet containing no Doxycycline at 4 months of age). The cells were stained with antibodies to CXCR5 or BLR-1 (Burkitt's Lymphoma receptor 1), or CCR7 and analyzed by flow cytometry.

FIG. 12 is a graph showing suppression of tumor growth in FLL and DLBCL mice by pharmacological agents. Tumor cells were harvested from lymph nodes and spleens and transplanted. The recipient mice were held until tumors became clinically apparent. Tumor recipient (clear bars) and wild type (filled bars) mice then received daily injections of the indicated drugs for 7 days of either cyclosporine A (csa), FK506, rapamycin (rap) or cyclophosphamide (cyph). The mice were euthanized 24 hours after the last injection of drug, and lymph nodes were harvested for analysis of either total number of cells or surface markers of donor cells (not shown).

FIG. 13 is a graph showing suppression of tumor growth by pharmacological agents in tumors derived from quadruply transgenic mice with adult onset lymphoma. Tumor cells were harvested from lymph nodes and spleens and transplanted as described. The recipient mice were held until tumors became clinically apparent. The mice were then either maintained on a normal mouse diet, or switched on to a diet containing doxycycline, that shut off MYC overexpression. The mice were euthanized at the indicated times, and the lymph nodes were collected, used to generate single cell suspensions, cell counts and flow cytometric analysis.

FIG. 14 is a digital image showing the histological appearance of the tumorous spleens collected from mice that were withdrawn from doxycycline completely at 4 months of age (DLBCL-like), or switched from a dose of 200 mg/kg to one of 50 mg/kg of doxycycline.

FIG. 15 is a digital image showing the conversion of FLL into DLBCL tumors. Histological appearance of tumorous spleens collected from mice that were maintained on 200 mg/kg of doxycycline throughout the experiment (MW-tTA Q), switched to 50 mg/kg of doxycycline at four months of age, and maintained in that dose until they were euthanized, or mice that were switched to a dose of doxycycline of 20 mg/kg upon initial presentation of clinical signs associated with lymphoma.

FIG. 16 is a digital image showing the validation of MYC protein level following titration of doxycycline in vivo. Splenic B-cells were obtained from mice that were maintained on a high dose of oral doxycycline (200 mk/kg), or normal food (No Dox), or were maintained at either 20 mg/lg or 50 mg/kg of doxycycline). The cells were lysed using a 0.1% triton X-100 buffer and used for western blot analysis. The blots were probed with an antibody to MYC (recognizes human and mouse forms), or stripped and re-probed with an antibody to β-actin, as a loading control.

FIG. 17 is a graph showing the establishment of murine BL by intrinsic antigen. Primary transplants. Spleen and lymph node cells were harvested from an Eμ-MYC/BCR^{HEL} mouse at 4 weeks of age. Cells from spleen and lymph nodes were pooled at a 1:1 ratio, and 10⁶ cells were introduced into either syngeneic wild type mice (empty circles) or sHEL transgenic mice (filled circles) by intravenous injection. Tissues were collected at indicated time points from lymph nodes and analyzed for total number of cells. Samples taken from wild type mice were analyzed at the same times (empty squares). Values in Y-axis represent millions of cells (×10⁶).

FIG. 18 is a graph showing the maintenance of murine BL by intrinsic antigen. Secondary transplants. Spleen and lymph node cells were collected from the mice whose cell counts are shown in FIG. 17, 16 days after transplantation. Cells from spleen and lymph nodes were pooled at a 1:1 ratio, and 10⁵ cells were introduced into either wild type recipients (empty circles) or sHEL transgenic mice (filled circles) by intravenous injection. Cells were collected from 6 peripheral lymph nodes (pairs of ingunal, axillary and brachial nodes) at the indicated times after the transplantation and analyzed as in FIG. 17. Values in Y-axis represent millions of cells (×10⁶).

FIGS. 19A-19D is a series of graphs showing the detection of multiple immunoglobulin isotypes in the serum of tumor-bearing Eμ-MYC/VDJki/Lt-Tg/sHEL mice. Sera were obtained from groups of 8 mice of the specified genotypes and assayed in triplicate by ELISA against HEL, using isotype-specific secondary antibodies (FIG. 19A, IgM; FIG. 19B, IgG1; FIG. 19C, IgG2; FIG. 19D, IgG3; FIG. 19E, IgA). The sera were obtained from wild type mice lane 1, naïve BCR^{HEL} mice lane 2, BCR^{HEL} that were immunized with HEL emulsified in complete Freund's adjuvant 14 days prior to bleeding lane 3, naïve VDJki/Lt-Tg mice lane 4, VDJki/Lt-Tg mice that were immunized with HEL emulsified in complete Freund's adjuvant 14 days prior to bleeding, tumor-bearing lane 5, Eμ-MYC/BCR^{HEL}/sHEL mice lane 6, or tumor bearing Eμ-MYC/VDJki/Lt-Tg/sHEL mice lane 7. The results shown here are from one representative experiment of three.

FIGS. 20A-20C are graphs showing the suppression of tumor growth by drugs (FIG. 20A, Eμ-MYC tumors; FIG. 20B, Eμ-MYC/BCR^{HEL}/sHEL tumors; FIG. 20C, tumors obtained from the jaw of a 6 week old MMTV-rtTA/TRE-MYC/BCR^{HEL}/sHEL mouse. Tumor cells were harvested from lymph nodes and spleens and transplanted as described in FIG. 17. The recipient mice were held until tumors became clinically apparent. Tumor recipient (clear bars) and wild type (filled bars) mice then received daily injections of the indicated drugs for 7 days. All mice were euthanized 24 hours after the last injection of drug, and lymph nodes were harvested for analysis of either total number of cells. A. The drugs used in these experiments were cyclosporin A (CsA), FK506, rapamycin (rap), and cyclophosphamide (cyph).

FIGS. 21A-21D are graphs showing the suppression of proliferation and survival of normal and transformed B-lymphocytes. B-cells were obtained from the spleens of either BCR^{HEL} mice (FIGS. 21A and 21B), or tumor bearing Eμ-MYC/BCR^{HEL}/sHEL mice (FIGS. 21C and 21D). These cells were then cultured in the presence of antibodies to IgM and CD40 (FIGS. 21A and 21B), or in media alone (FIGS. 21C and 21D), with the indicated amounts of drugs. Proliferation was determined as counts per minute (c.p.m.) of H³-thymidine that were incorporated following a 12-hour pulse. Apoptosis was determined by flow cytometric analysis of 7AAD staining.

FIGS. 22A-22F are cytometric analysis images and graphs showing the suppression of proliferation and survival of murine and human BL cell lines. B-cell lines were established from cells obtained from an Eμ-MYC/BCR^{HEL}/sHEL tumorous lymph node. These cells grow in lymphocyte media, and appear to be blasting, as determined by their forward and side scatter characteristics. Data is shown for one representative cell line, TBL-4 (FIG. 22A). These cells also retain expression of B220 and BCR^{HEL} on their surface (FIG. 22B). TBL-4 cells (FIGS. 22C and D), or the human BL cell line Raji cells (FIGS. 22E and F) were cultured with the indicated amounts of drugs. Proliferation was determined as counts per minute (c.p.m.) of H³-thymidine that were incorporated following a 12-hour pulse. Apoptosis was determined by flow cytometric analysis of 7AAD staining. Similar observations were obtained with five other murine BL-cell lines, as well as with two other human BL cell lines, Ramos and Daudi (not shown).

FIGS. 23A-23C are digital images showing the biochemical analysis of early BCR signaling in normal activated B-cells and BL tumor cells. (FIG. 23A, Cell lysates were obtained from BCR^{HEL} B-cells activated for 12 hours (lane A) or 24 hours (lane B), or a tumor obtained form an Eμ-MYC/BCR^{HEL}/sHEL mouse (lane C), or a tumor obtained from a MMTV-rtTA/TRE-MYC/BCR^{HEL}/SHEL mouse (lane D). The lysates were run on a 10% SDS gel and probed with antibodies to Syk (upper panel) or Actin (lower panel)). (FIG. 23B. Lysates were obtained from four different tumors obtained in Eμ-MYC/BCR^{HEL}/sHEL mice (lanes A-D), or BCR^{HEL} B-cells that were activated for 24 hours (lane E)). The lysates were run on 12% SDS-PAGE gels and probed with antibodies to phospho-Lyn (Y508), phospho-Syk (Y525/526), phospho-BLNK (Y96) and actin, or phospho-tyrosine (4G10) (FIG. 23C).

FIG. 24 is a series of flow cytometry images showing that the shRNA-mediated knockdown of Syk confers a competitive disadvantage to established murine BL cell lines.

FIG. 25A is a flow cytometry image and FIG. 25B is a digital image showing the validation of the Syk-specific shRNAs. FIG. 25A represents the GFP expression level in the non-transduced TBL-8 cells (filled histogram), the GFP+ sorted cells, harboring an intact, and inactive pSICO virus, or the transduced cells that had been sorted and cultured in the presence of 40HT for 48 hours. FIG. 25B shows the result of the western blot analysis for Syk protein level from cell lysates obtained in the cells represented in FIG. 25A.

FIG. 26 is a graph showing that Syk expression is required for the maintenance of B-cell lymphomas.

FIGS. 27A and 27B show the in vivo effects of shRNA-mediated Syk knockdown on the competitive fitness of murine B-CLL tumors. FIG. 27A shows photographs in either brightfield, or with GFP fluorescence for a representative spleen and tumor nodules that form on the liver of tumor-bearing Rag-1−/− mice. The summary of the flow cytometric data is presented in FIG. 27B.

FIGS. 29A-29D are graphs showing the response of murine and human BL cell lines to the Syk pharmacological inhibitor R408. 2×10⁴ cells from the following murine BL cell lines: TBL-1, TBL-8, TBL-9, TBL-14 (FIGS. 29A and 29B), or the human cell lines (Raji, Ramos, Daudi; FIGS. 29C and 29D) in increasing concentrations of either a pharmacological inhibitor of Syk (R408; FIGS. 29A and 29C), or an inactive chemical analogue (FIGS. 29B and 29D), as a negative control.

FIG. 29 is a graph showing the specificity of Syk inhibitor R406 to murine B-cell lymphomas.

FIG. 30 is a schematic drawing showing a protocol for testing of drug candidates in vivo, using mouse models of NHL of the invention.

FIG. 31 is a graph showing that pharmacological inhibition of Syk in B-cell lymphomas that do not express a B cell receptor on the surface (resemble pre-B cell Acute lymphocytic leukemia/lymphoma of humans) does not extend the survival of tumor-bearing mice.

FIGS. 32A and 32B, are graphs showing that pharmacological inhibition of Syk in mice harboring murine B-CLL tumors caused significant remission (FIG. 32A) and extended lifespan (FIG. 32B) of treated tumor-bearing mice.

FIG. 33 is a graph showing that pharmacological inhibition of Syk in mice harboring murine BL tumors caused significant remission and extended lifespan of treated tumor-bearing mice.

FIGS. 34A and 34B are graphs showing that human BL cell lines undergo apoptosis in vitro following pharmacological inhibition of Syk.

### DETAILED DESCRIPTION OF THE INVENTION

The present invention generally relates to non-human animal models, and preferably, rodent models, and more preferably, mouse models, of B-cell Non-Hodgkin's Lymphoma (NHL). In particular, the present invention provides animal models of B cell NHL including, B cell chronic lymphocytic leukemia/lymphoma (B-CLL), Burkitt's lymphoma (BL), Follicular-like lymphoma (FLL) and Diffuse large B-cell lymphoma (DLBCL), as well as various methods for producing these non-human animal models. These animal models, as well as cell lines produced from or derived from these models, are useful tools for a variety of methods, including, but not limited to, preclinical testing of drug candidates, and particularly drug candidates that are specific for human proteins, and any research, development, pharmaceutical, or clinical purpose, including but not limited to, the identification, development, and/or testing of drugs (therapeutics, prophylactics, etc.), targets, markers, and/or research tools for use in the diagnosis of, study of, or treatment of any Non-Hodgkin's Lymphoma, such as those described herein, or for any related condition.

The present invention also relates to the targeting of the tec tyrosine kinase, Syk, for the prophilaxis and treatment of B cell NHL, including B cell chronic lymphocytic leukemia/lymphoma (B-CLL), Burkitt's lymphoma (BL), Follicular-like lymphoma (FLL) and Diffuse large B cell lymphoma (DLBCL). Although the invention is described below with particular reference to murine (mouse) models, it is to be understood that the invention can be applied to other rodent models, and to other non-human animals, as desired. In addition, while various embodiments of the present invention have been described in detail, it is apparent that modifications and adaptations of those embodiments will occur to those skilled in the art, including modifications to the genes, constructs, and method steps described herein. It is to be expressly understood, however, that such modifications and adaptations are within the scope of the present invention.

General Definitions

The practice of the present invention will employ, unless otherwise indicated, conventional techniques of molecular biology (including recombinant techniques), microbiology, cell biology, biochemistry, nucleic acid chemistry, and immunology, which are well known to those skilled in the art. Such techniques are explained fully in the literature, such as, Molecular Cloning: A Laboratory Manual, second edition (Sambrook et al., 1989) and Molecular Cloning: A Laboratory Manual, third edition (Sambrook and Russel, 2001), jointly referred to herein as “Sambrook”); Current Protocols in Molecular Biology (F. M. Ausubel et al., eds., 1987, including supplements through 2001); PCR: The Polymerase Chain Reaction, (Mullis et al., eds., 1994); Harlow and Lane (1988) Antibodies, A Laboratory Manual, Cold Spring Harbor Publications, New York; Harlow and Lane (1999) Using Antibodies: A Laboratory Manual Cold Spring Harbor Laboratory Press, Cold Spring Harbor, N.Y. jointly referred to herein as “Harlow and Lane”), Beaucage et al. eds., Current Protocols in Nucleic Acid Chemistry John Wiley & Sons, Inc., New York, 2000); “Manipulating the Mouse Embryo” (Andras Nagy, Marina Gertsenstein, Cold Spring Harbor Laboratory Press, 2003, incorporated herein by reference in its entirety), “Gene Targeting: A Practical Approach” (Edited by Alexandra A. Joyner, Oxford University Press, 2000), “Mouse Genetics and Transgenics: A Practical Approach” (Ian J. Jackson and Catherine A. Abbott, editors, Oxford University Press, 2000), “The Laboratory Mouse: Handbook of Experimental Animals”, Hans Hedrich, editor, Elselvier Academic Press, 2004), “Transgenic Animals” (F. Grosveld and G. Kollias, editors, Academic Press; 1st edition, 1992), or “Transgenic Animal Technology: a Laboratory Handbook (2nd Ed.)” (Carl A. Pinkert, Lavoisier, 2002).

According to the present invention, the general use herein of the term “antigen” refers: to any portion of a protein (peptide, partial protein, full-length protein), wherein the protein is naturally occurring or synthetically derived, to a cellular composition (whole cell, cell lysate or disrupted cells), to an organism (whole organism, lysate or disrupted cells) or to a carbohydrate (such as those expressed on cancer cells), or other molecule, or a portion thereof. An antigen elicits an antigen-specific immune response (e.g., a humoral and/or a cell-mediated immune response) against the same or similar antigens that are encountered within the cells and tissues of an individual to which the antigen is administered. Alternatively, an antigen can act as a toleragen.

According to the present invention, the phrase “selectively binds to” refers to the ability of a protein (e.g., an antigen or an antibody) to preferentially bind to another specified protein or proteins. More specifically, the phrase “selectively binds” refers to the specific binding of one protein to another, wherein the level of binding, as measured by any standard assay (e.g., an immunoassay), is statistically significantly higher than the background control for the assay. For example, when performing an immunoassay, controls typically include a reaction well/tube that contain an antibody or receptor (a BCR) (i.e., in the absence of antigen), wherein an amount of reactivity (e.g., non-specific binding to the well) by the antibody or receptor in the absence of the antigen is considered to be background. Binding can be measured using a variety of methods standard in the art including enzyme immunoassays (e.g., ELISA), immunoblot assays, etc.).

Reference to an isolated protein or polypeptide in the present invention includes full-length proteins, fusion proteins, or any fragment, domain, conformational epitope, or homologue of such proteins. More specifically, an isolated protein, according to the present invention, is a protein (including a polypeptide or peptide) that has been removed from its natural milieu (i.e., that has been subject to human manipulation) and can include purified proteins, partially purified proteins, recombinantly produced proteins, and synthetically produced proteins, for example. As such, “isolated” does not reflect the extent to which the protein has been purified. Preferably, an isolated protein of the present invention is produced recombinantly. According to the present invention, the terms “modification” and “mutation” can be used interchangeably, particularly with regard to the modifications/mutations to the amino acid sequence of proteins or portions thereof (or nucleic acid sequences) described herein.

As used herein, the term “homologue” is used to refer to a protein or peptide which differs from a naturally occurring protein or peptide (i.e., the “prototype” or “wild-type” protein) by minor modifications to the naturally occurring protein or peptide, but which maintains the basic protein and side chain structure of the naturally occurring form. Such changes include, but are not limited to: changes in one or a few amino acid side chains; changes one or a few amino acids, including deletions (e.g., a truncated version of the protein or peptide) insertions and/or substitutions; changes in stereochemistry of one or a few atoms; and/or minor derivatizations, including but not limited to: methylation, glycosylation, phosphorylation, acetylation, myristoylation, prenylation, palmitation, amidation and/or addition of glycosylphosphatidyl inositol. A homologue can have either enhanced, decreased, or substantially similar properties as compared to the naturally occurring protein or peptide. A homologue can include an agonist of a protein or an antagonist of a protein. Homologues can be produced using techniques known in the art for the production of proteins including, but not limited to, direct modifications to the isolated, naturally occurring protein, direct protein synthesis, or modifications to the nucleic acid sequence encoding the protein using, for example, classic or recombinant DNA techniques to effect random or targeted mutagenesis.

A homologue of a given protein may comprise, consist essentially of, or consist of, an amino acid sequence that is at least about 45%, or at least about 50%, or at least about 55%, or at least about 60%, or at least about 65%, or at least about 70%, or at least about 75%, or at least about 80%, or at least about 85%, or at least about 90%, or at least about 95% identical, or at least about 95% identical, or at least about 96% identical, or at least about 97% identical, or at least about 98% identical, or at least about 99% identical (or any percent identity between 45% and 99%, in whole integer increments), to the amino acid sequence of the reference protein. In one embodiment, the homologue comprises, consists essentially of, or consists of, an amino acid sequence that is less than 100% identical, less than about 99% identical, less than about 98% identical, less than about 97% identical, less than about 96% identical, less than about 95% identical, and so on, in increments of 1%, to less than about 70% identical to the naturally occurring amino acid sequence of the reference protein.

As used herein, unless otherwise specified, reference to a percent (%) identity refers to an evaluation of homology which is performed using: (1) a BLAST 2.0 Basic BLAST homology search using blastp for amino acid searches and blastn for nucleic acid searches with standard default parameters, wherein the query sequence is filtered for low complexity regions by default (described in Altschul, S. F., Madden, T. L., Schääffer, A. A., Zhang, J., Zhang, Z., Miller, W. & Lipman, D. J. (1997) “Gapped BLAST and PSI-BLAST: a new generation of protein database search programs.” Nucleic Acids Res. 25:3389-3402, incorporated herein by reference in its entirety); (2) a BLAST 2 alignment (using the parameters described below); (3) and/or PSI-BLAST with the standard default parameters (Position-Specific Iterated BLAST. It is noted that due to some differences in the standard parameters between BLAST 2.0 Basic BLAST and BLAST 2, two specific sequences might be recognized as having significant homology using the BLAST 2 program, whereas a search performed in BLAST 2.0 Basic BLAST using one of the sequences as the query sequence may not identify the second sequence in the top matches. In addition, PSI-BLAST provides an automated, easy-to-use version of a “profile” search, which is a sensitive way to look for sequence homologues. The program first performs a gapped BLAST database search. The PSI-BLAST program uses the information from any significant alignments returned to construct a position-specific score matrix, which replaces the query sequence for the next round of database searching. Therefore, it is to be understood that percent identity can be determined by using any one of these programs.

Two specific sequences can be aligned to one another using BLAST 2 sequence as described in Tatusova and Madden, (1999), “Blast 2 sequences—a new tool for comparing protein and nucleotide sequences”, FEMS Microbiol Lett. 174:247-250, incorporated herein by reference in its entirety. BLAST 2 sequence alignment is performed in blastp or blastn using the BLAST 2.0 algorithm to perform a Gapped BLAST search (BLAST 2.0) between the two sequences allowing for the introduction of gaps (deletions and insertions) in the resulting alignment. For purposes of clarity herein, a BLAST 2 sequence alignment is performed using the standard default parameters as follows.

For blastn, using 0 BLOSUM62 matrix:

Reward for match=1

Penalty for mismatch=−2

Open gap (5) and extension gap (2) penalties

gap x_dropoff (50) expect (10) word size (11) filter (on)

For blastp, using 0 BLOSUM62 matrix:

Open gap (11) and extension gap (1) penalties

gap x_dropoff (50) expect (10) word size (3) filter (on).

An isolated nucleic acid molecule is a nucleic acid molecule that has been removed from its natural milieu (i.e., that has been subject to human manipulation), its natural milieu being the genome or chromosome in which the nucleic acid molecule is found in nature. As such, “isolated” does not necessarily reflect the extent to which the nucleic acid molecule has been purified, but indicates that the molecule does not include an entire genome or an entire chromosome in which the nucleic acid molecule is found in nature. An isolated nucleic acid molecule can include a gene. An isolated nucleic acid molecule that includes a gene is not a fragment of a chromosome that includes such gene, but rather includes the coding region and regulatory regions associated with the gene, but no additional genes that are naturally found on the same chromosome. An isolated nucleic acid molecule can also include a specified nucleic acid sequence flanked by (i.e., at the 5′ and/or the 3′ end of the sequence) additional nucleic acids that do not normally flank the specified nucleic acid sequence in nature (i.e., heterologous sequences). Isolated nucleic acid molecule can include DNA, RNA (e.g., mRNA), or derivatives of either DNA or RNA (e.g., cDNA). Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a protein or domain of a protein.

A recombinant nucleic acid molecule is a molecule that can include at least one of any nucleic acid sequence encoding any one or more proteins described herein operatively linked to at least one of any transcription control sequence capable of effectively regulating expression of the nucleic acid molecule(s) in the cell to be transfected. Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a protein. In addition, the phrase “recombinant molecule” primarily refers to a nucleic acid molecule operatively linked to a transcription control sequence, but can be used interchangeably with the phrase “nucleic acid molecule”.

One type of recombinant nucleic acid molecule is a transgene. A transgene is a gene or genetic material which has been transferred by any of a number of genetic engineering techniques from one organism to another.

A recombinant nucleic acid molecule includes a recombinant vector, which is any nucleic acid sequence, typically a heterologous sequence, which is operatively linked to the isolated nucleic acid molecule encoding a fusion protein of the present invention, which is capable of enabling recombinant production of the fusion protein, and which is capable of delivering the nucleic acid molecule into a host cell according to the present invention. Such a vector can contain nucleic acid sequences that are not naturally found adjacent to the isolated nucleic acid molecules to be inserted into the vector. The vector can be either RNA or DNA, either prokaryotic or eukaryotic, and preferably in the present invention, is a virus or a plasmid. Recombinant vectors can be used in the cloning, sequencing, and/or otherwise manipulating of nucleic acid molecules, and can be used in delivery of such molecules (e.g., as in a DNA vaccine or a viral vector-based vaccine). Recombinant vectors are preferably used in the expression of nucleic acid molecules, and can also be referred to as expression vectors. Preferred recombinant vectors are capable of being expressed in a transfected host cell.

In a recombinant molecule of the present invention, nucleic acid molecules are operatively linked to expression vectors containing regulatory sequences such as transcription control sequences, translation control sequences, origins of replication, and other regulatory sequences that are compatible with the host cell and that control the expression of nucleic acid molecules of the present invention. In particular, recombinant molecules of the present invention include nucleic acid molecules that are operatively linked to one or more expression control sequences. The phrase “operatively linked” refers to linking a nucleic acid molecule to an expression control sequence in a manner such that the molecule is expressed when transfected (i.e., transformed, transduced or transfected) into a host cell.

According to the present invention, the term “transfection” is used to refer to any method by which an exogenous nucleic acid molecule (i.e., a recombinant nucleic acid molecule) can be inserted into a cell. The term “transformation” can be used interchangeably with the term “transfection” when such term is used to refer to the introduction of nucleic acid molecules into microbial cells, such as algae, bacteria and yeast. In microbial systems, the term “transformation” is used to describe an inherited change due to the acquisition of exogenous nucleic acids by the microorganism and is essentially synonymous with the term “transfection.” Therefore, transfection techniques include, but are not limited to, transformation, chemical treatment of cells, particle bombardment, electroporation, microinjection, lipofection, adsorption, infection and protoplast fusion.

Animal Models Exemplifying the Invention

The present invention discloses the generation of a variety of non-human animal models, exemplified by mouse models, of large B cell lymphomas (Non-Hodgkin's Lymphomas, or NHLs), which are MYC-driven and dependent upon antigenic stimulation for their maintenance, or alternatively, which are MYC-driven and have tonic or constitutive signals through their B cell receptors (BCRs). These mouse models can be engineered to either express human proteins, or to replace the endogenous murine protein with a human counterpart, in order to directly test drug candidates, including small molecules, antibodies, proteins and nucleic acids, for their efficacy in vivo, in a preclinical setting, for example.

The invention specifically provides various animal models for each of the following forms of NHL: B cell chronic lymphocytic leukemia/lymphoma (B-CLL), Burkitt's lymphoma (BL), Follicular-like lymphoma (FLL) and Diffuse large B-cell lymphoma (DLBCL). While each model is produced using different combinations of genetic manipulations and in some cases, chemical manipulation, certain aspects of these animal models are shared among the models, as discussed below.

First, in all of the animal models described herein, the animal overexpresses MYC. The MYC gene encodes a 64-kDa transcription factor that is expressed in many tissues (Boxer and Dang, (1999) Oncogene 20, 5595-5610). MYC was originally identified as the cellular progenitor of the viral oncogene, v-myc, and overexpression of MYC has been implicated in many human tumors (Nesbit et al., (1999) Oncogene 13, 3004-3016; Pelengaris et al., (2002) Nat. Rev. Cancer 2, 764-776). Prominent among these tumors are diverse forms of lymphoma (Look, A. T. (1997) Science 278, 1059-1064). Accordingly, the normal function of MYC has important roles in the development, proliferation, and survival of lymphocytes. The nucleic acid sequence and amino acid sequence of MYC are well known in the art for a variety of animal species. The nucleic acid sequence of murine MYC is represented herein by SEQ ID NO:1. SEQ ID NO:1 encodes the amino acid sequence of murine MYC represented herein by SEQ ID NO:2. The nucleic acid sequence of human MYC is represented herein by SEQ ID NO:3. SEQ ID NO:1 encodes the amino acid sequence of human MYC represented herein by SEQ ID NO:4.

Indeed, any nucleic acid molecule encoding MYC that can be overexpressed in a non-human animal model of the invention (or progeny thereof) is encompassed by the invention, with preference given to MYC nucleotides from the same species as the animal model. Any nucleic acid molecule overexpressing MYC can be used in any of the animal models described herein, although some preferred nucleic acid molecules (transgenes) are described. Several transgenes for achieving MYC overexpression are known in the art and are described herein. It is not necessary to produce a MYC-overexpressing transgenic animal de novo, since any MYC-overexpressing animal may be used with the current invention (although de novo production of a transgenic animal for use in the invention can readily be done, if desired, using common techniques for the production of transgenic animals that are well known in the art). Particularly useful animals overexpress MYC predominantly in the B cell population (B cell compartment), such as, for example, the Eμ-MYC mouse strain described by Adams et al. (1985) Nature 318, 533-538, which is a commercially available mouse (Jackson Laboratories), or the MMTV-rtTA/TRE-MYC mouse strain, which is a double transgenic mouse produced by cross-breeding the MMTV-rtTA transgenic mouse and the TRE-MYC mouse (mice described in Felsher and Bishop, (1999) Mol. Cell 4, 199-207; Hennighausen et al., (1995) J. Cell Biochem. 59, 463-472). These transgenes can be used in any of the models of the invention.

In one embodiment, the MYC transgene comprises a nucleic acid sequence encoding MYC coupled to an Ig heavy chain enhancer. The Ig heavy chain enhancer results in overexpression of the gene primarily in the B cell compartment. The MYC transgene described herein and known in the art as Eμ-MYC is such a nucleic acid sequence. The Eμ-MYC transgene and a mouse that expresses this transgene is described in detail in Adams et al. (1985) Nature 318, 533-538. These transgenes can be used in any of the models of the invention.

In one aspect of the invention, animals that overexpress MYC in an inducible, controllable manner are used. In these animals, MYC overexpression can be regulated in a temporal manner, e.g., suppressed (repressed) until a specified time point. The control of MYC expression can be regulated by any suitable technique, which includes repression or induction through the use of compounds that repress or activate, respectively, transcription of the transgene encoding MYC. For example, the MMTV-rtTA/TRE-MYC mouse strain mentioned above and known in the art may be administered doxycycline or tetracycline to repress MYC expression, and MYC expression can be commenced by removing the doxycycline or tetracycline from the diet of the mice. Furthermore, the level of MYC overexpression can be regulated by modulating the amount of repressor used, which becomes important for the inventors' discoveries related to the NHL animal models. Many additional inducible gene expression/repression systems suitable for use in the present invention are known in the art.

In one aspect, the MYC transgene comprises a nucleic acid sequence encoding MYC coupled to an inducible expression control sequence, wherein B cell-specific expression of the MYC transgene can be selectively repressed. For example, the MYC transgene can include a nucleic acid sequence encoding MYC coupled to a tetracycline (TET) responsive expression control element (TRE), the expression of which is controlled by a B-cell specific TET-off repressor. An example of a nucleic acid sequence encoding MYC coupled to a TRE is exemplified in the transgene known as TRE-MYC. A nucleic acid sequence for the TRE-MYC transgene is described in detail in Felsher and Bishop, (1999) Mol. Cell 4, 199-207, along with a mouse that is expresses this transgene. An example of a B-cell specific TET-off repressor is a mouse mammary tumor virus long term repeat (MMTV-LTR) driving expression of reverse tetracycline-dependent transactivator (rtTA) (MMTV-rtTA). A nucleic acid sequence for MMTV-rtTA transgene is described in detail in Hennighausen et al., (1995) J. Cell Biochem. 59, 463-472, along with a mouse that expresses this transgene. In the case of the gene coupled to the TET repressor, the expression of the gene can be regulated by provision (or withdrawal) of a compound that regulates the repressor, which includes tetracycline, doxycycline, and functional derivatives thereof. These transgenes can be used in any of the models of the invention.

In the animal models of the invention, the overexpression of MYC is done in the context of the concurrent overexpression of a defined B cell receptor (BCR), and in most of the models (but not B-CLL), in the context of a defined autoreactive B cell background (e.g., the BCR binds to an antigen that is an autoantigen, or that is recognized as a “self” or “autoantigen” by the animal into which the BCR transgene is placed). The defined autoreactive B cell background can be achieved by a variety of methods. For example, a defined B cell receptor is most typically provided by overexpressing a BCR transgene (or transgenes whose products combine to form a BCR), so that the BCR is expressed abundantly or exclusively on the surface of B cells in the animal. In one aspect of this embodiment, the BCR can be a pre-rearranged BCR. In another aspect of this embodiment, the BCR can be provided in the form of a BCR VDJ prearranged transgene that can be introduced into the Ig heavy chain locus of the animal (a “knock-in”) along with a second transgene that encodes the corresponding Ig light chain, wherein the antigen specificity of the resulting BCR is defined, and wherein BCR VDJ rearrangement and isotype switching can be identified and evaluated, if desired. Both of these types of BCRs are exemplified herein. These transgenes can be used in any of the models of the invention.

An example of a transgene encoding a prerearranged BCR of defined antigen specificity is the BCR^{HEL} transgene, which is a pre-rearranged BCR that selectively binds to hen egg lysozyme (HEL). A nucleic acid sequence for the BCR^{HEL} transgene is described in Goodnow et al., (1988) Nature 334, 676-682), along with a description of a mouse that expresses this transgene. These transgenes can be used in any of the models of the invention.

The present invention also provides an example of two transgenes that when expressed, produce a BCR that recognizes HEL, and that can be further evaluated for affinity maturation through somatic mutation and clonal selection in the germinal center (GC), as well as isotype switching, can be identified and evaluated. A nucleic acid sequence for a transgene identified herein as VDJki, which encodes a portion of a heavy chain of a BCR that recognizes HEL and is designed to be knocked into the Ig heavy chain locus of a mouse, is described in Pape et al., (2003) J Exp Med 197, 1677-87, along with a description of a mouse that expresses this transgene. A nucleic acid sequence for a transgene that encodes a lambda light chain (denoted Lt-tg) that recognizes HEL is described in Pape et al., (2003) J Exp Med 197, 1677-87, along with a description of a mouse that expresses this transgene. These transgenes can be used in any of the models of the invention.

In one embodiment, the BCR selectively binds to (is specific for, recognizes) any antigen that is or “becomes” a self antigen or an autoantigen because it is expressed by the animal model via introduction of another transgene that expresses the antigen. For example, in the animals exemplified herein, a pre-rearranged BCR transgene is expressed that recognizes the well-known antigen, hen egg lysozyme (HEL) (e.g., refer to the above-described BCR^{HEL} transgene or the VDJki/Lt-tg combination). However, HEL is not an antigen that is endogenously expressed by a mouse, for example, and so to create the autoreactive B cell environment described above, a transgene encoding HEL is also expressed in the animal. In the examples described herein, the HEL is soluble HEL (sHEL) and is ubiquitously expressed or expressed so that it is available systemically in the animal. A nucleic acid sequence for a transgene that encodes sHEL is described in Goodnow et al., (1988) Nature 334, 676-682), along with a description of a mouse that expresses this transgene. However, it is to be understood that a variety of other BCR/antigen combinations can be used in this embodiment, including antigens that are soluble or membrane-expressed antigens. These transgenes can be used in any of the models of the invention.

In another embodiment of the invention, a particular BCR antigen specificity is chosen because the antigen recognized by the BCR is an endogenous antigen expressed by the animal (i.e., in this case, the BCR is naturally an autoreactive BCR because it recognizes as an antigen a protein that is endogenously expressed by the animal). In this aspect, the autoreactivity can be due to the presence in the animal of the primary antigen recognized by the BCR or due to cross-reactivity of a BCR that otherwise recognizes a “foreign” or “non-self” antigen with an antigen that is endogenously expressed by the host animal. This embodiment may be preferred in some circumstances, because fewer transgenes are required to be introduced into the animal model, facilitating production of the animals, and allowing for a more natural autoreactive environment which may have advantages for comparison to naturally occurring NHL. As one non-limiting example of such a BCR, the inventors describe herein the use of a transgene denoted ARS.A1, which recognizes arsenate, but can also become anergic in response to binding to a low-affinity self-antigen present in the mouse (ssDNA), and in the case of the animal models described herein, creates the prescribed autoreactive B cell environment by introduction of only the BCR transgene (i.e., a transgene for the antigen is not required). A nucleic acid sequence for the transgene known as ARS.A1 is described in Benschop et al., (2001) Immunity 14, 33-43, along with a description of a mouse that expresses this transgene. These transgenes can be used in any of the models of the invention.

Another aspect of the invention related to certain animal models of the invention relates to animals that overexpress MYC in an inducible (or repressible) manner, wherein the timing of the repression of MYC expression is critical to the induction of a particular form of NHL in the animal. The inventors have surprisingly discovered that changes in the regulation of the timing of expression and the level of expression of MYC in an autoreactive B cell environment in genetically identical mice will result in different forms of NHL that closely mimic their corresponding human forms. Therefore, the inventors have created a controlled, powerful system for investigating differences in different NHL forms, which can be used to identify unique targets specific to one form of the NHL and not another, and to identify highly specific therapeutic compounds for treatment of specific forms of NHL.

More specifically, the present inventors have established that in an animal model that overexpresses MYC in an inducible (or repressible) manner in an autoreactive B cell background (described above), if the expression of the MYC transgene is not expressed in the transgenic animals as of the birth of the animal (no repression is applied, or induction is continuously provided), the animals will develop a form of NHL that closely mimics human Burkitt's Lymphoma (BL; see Example 2). In contrast, if the expression of MYC is repressed in the genetically same animals from birth until adulthood (in mice, this occurs at approximately 4 months of age), at which time the animals are not released from repression of MYC, but rather, a low level of MYC expression is allowed by reduction in the amount of the repressive agent (in the examples, doxycycline in the diet), the animals will develop an NHL that closely mimics human Follicular-like lymphoma (FLL), instead of BL. Alternatively, if the if the expression of MYC is repressed in the genetically same animals from birth until adulthood, at which point repression of MYC is completely released (the animals are removed from doxycycline diets, in the examples provided herein), the animals will develop a form of NHL that closely mimics Diffuse large B-cell lymphoma (DLBCL), instead of BL or FLL. This discovery is remarkable, and completely surprising.

The present invention also includes panels of non-human animal models, including panels of NHL non-human animal models that include any one, two, three, four, five, six, seven, eight, or more of the non-human animal models of NHL of the invention. The panel of animal models may include one or more of any other animal model of NHL not encompassed by the present invention, any animal model of a related disease or condition, or any animal model for an unrelated disease or condition, or any control animal (negative or positive). Such panels of animal models are useful in any of the methods described herein (any research, development, pharmaceutical, or clinical purpose, including but not limited to, the identification, development, and/or testing of drugs (therapeutics, prophylactics, etc.), targets, markers, and/or research tools for use in the diagnosis of, study of, and/or treatment of any Non-Hodgkin's Lymphoma, such as those described herein, or for any related condition). In particular, the panels of animal models, since they may animal models of different forms of NHL described herein, can be extremely valuable in identifying drug candidates that are specific for a particular form or forms of NHL, and/or for differentiating the causes of, targets of, diagnosis of, and/or therapies for a particular form or forms of NHL.

The present invention also includes an egg containing the transgenes and genetic modifications described herein, wherein the egg is capable of maturing into a transgenic non-human animal of the present invention.

The present invention also includes any isolated part of any of the non-human animal models described herein, including any cell, any bodily fluid, and any tissue or organ, particularly those that contain the transgenes genetic modifications described herein.

Mouse Model for B-Cell Chronic Lymphocytic Leukemia/Lymphoma (B-CLL)

One embodiment of the invention relates to mouse models of MYC-driven, antigen-dependent B cell chronic lymphocytic Leukemia/Lymphoma (B-CLL), as well as methods to produce such mouse models, and methods of using such mouse models. Prior to the present invention, there was a lack of a good mouse model for B-CLL for the further understanding of the molecular and genetic basis of the pathogenesis of the disease, as well as the use of the model for as a preclinical tool for the development of novel therapies, and identification of therapeutically relevant targets. The present invention provides a solution to this need in the art by providing a mouse model of B-CLL that resembles the human disease in terms of the flow cytometric profile of the tumors, the sites of anatomical presentation, the histopathology, the grade of the disease (aggressiveness of the tumor), the anatomical site of MYC overexpression and age of onset. The key aspect for this model is the overexpression of oncogenes in the context of the overexpression of a B cell receptor (or tonic or constitutive expression of a B cell receptor). The mouse model is ready for use in testing compounds of pharmaceutical or research interest. In one embodiment, human proteins of interest will be retrovirally overexpressed in B-CLL tumors in order to directly determine the efficacy of anti-human monoclonal antibodies for preclinical use.

There are currently no mouse models that resemble B-CLL in any tangible manner. In particular, current mouse models do not give rise to tumors that satisfy the six criteria defined herein for B-CLL models (see Table 2). In particular, the B-CLL animal model of the invention mimics the human B-CLL disease on the level of relevant latency to onset, aggressiveness, anatomical presentation and evolution, histopathology, immunophenotype, and MYC overexpression. This mouse model, and various other aspects of this embodiment of the invention are described Example 1.

Accordingly, one embodiment of the invention relates to a non-human animal model of B cell chronic lymphocytic leukemia/lymphoma (B-CLL). This animal model of NHL is a transgenic non-human animal that expresses the following transgenes: (a) a MYC transgene that is overexpressed in the B cell lineage in the animal; and (b) a transgene encoding a pre-rearranged B cell receptor (BCR) that is overexpressed in the B cell lineage of the animal.

In one aspect of this embodiment, the MYC transgene comprises a nucleic acid sequence encoding MYC coupled to an Ig heavy chain enhancer. Such transgenes have been discussed above, and include, for example, Eμ-MYC. In one aspect of this embodiment, the MYC transgene comprises a nucleic acid sequence encoding MYC coupled to an inducible expression control sequence, wherein B cell-specific expression of the MYC transgene can be selectively repressed. For example, the MYC transgene can include a nucleic acid sequence encoding Myc coupled to a tetracycline (TET) responsive expression control element (TRE), the expression of which is controlled by a B-cell specific TET-off repressor, such as MMTV-rtTA. Such transgenes have been described above, and include, for example, TRE-MYC expressed in conjunction with MMTV-rtTA. Repression can be controlled for example by administration of a compound that regulates expression, such as tetracycline or doxycycline in the case of the MMTV-rtTA/TRE-MYC combination described herein.

In one aspect of this embodiment, the BCR transgene is expressed under the control of the endogenous immunoglobulin promoter. In one aspect, the BCR transgene selectively binds to hen egg lysozyme (HEL), such as the BCR^{HEL} transgene described herein.

In one aspect of this embodiment, the BCR transgene is expressed under the control of the endogenous immunoglobulin promoter. In one aspect, the transgene encodes a pre-rearranged VDJ region of an Ig heavy chain of a B cell receptor (BCR) that selectively binds to an antigen, wherein the transgene is designed to be knocked in to the Ig heavy chain locus of the animal. Such a transgene is exemplified by the VDJki transgene described herein. In addition to this knock-in, the animal expresses a transgene encoding a lambda light chain of the BCR that binds to the antigen of (b), exemplified by the transgene denoted Lt-tg and described herein.

Mice according to this embodiment of the invention can be produced by producing mice expressing one or more of each transgene, followed by cross-breeding, as needed to place all of the transgenes in the same genetic background, and/or by obtaining transgenic mice expressing one or more transgenes (e.g., commercially or from another source), and cross-breeding as necessary to place all of the transgenes in the same genetic background. The invention encompasses the parental transgenic lines and progeny thereof that continue to express the requisite transgenes.

Mouse Models for Burkitt's Lymphoma (BL)

One embodiment of the invention relates to mouse models of MYC-driven, antigen dependent Burkitt's Lymphoma, as well as methods to produce such mouse models, and methods of using such mouse models. Prior to the present invention, there was lack of a good mouse model for Burkitt's lymphoma for the further understanding of the molecular and genetic basis of the pathogenesis of the disease, as well as the use of the model for as a preclinical tool for the development of novel therapies, and identification of therapeutically relevant targets. The present invention provides a solution to this need in the art by providing several mouse models of Burkitt's lymphoma that resemble the human disease in terms of the flow cytometric profile of the tumors, the sites of anatomical presentation, the histopathology, the grade of the disease (aggressiveness of the tumor), the anatomical site of MYC overexpression, and age of onset (see Table 4). A key aspect for this model is the overexpression of oncogenes in the context of a defined autoreactive B-cell background. The mouse model is ready for use in testing compounds of pharmaceutical or research interest. In one embodiment, human proteins of interest will be retrovirally overexpressed in BL tumors in order to directly determine the efficacy of anti-human monoclonal antibodies for preclinical use.

The currently available models of Burkitt's Lymphoma (BL) rely on the overexpression of MYC early in the B-cell compartment (Eμ-MYC) in order to recapitulate the t8;14 translocation that juxtaposes the MYC ORF onto the IgH promoter/enhancer elements. Those mice give rise to Pre/Pro B-cell tumors that do not resemble BL. Moreover, the current mouse models do not give rise to tumors that satisfy the criteria defined herein for the present inventors' BL models. This mouse model, and various other aspects of this embodiment of the invention are described Example 2.

Accordingly, one embodiment of the invention relates to a non-human animal model of Burkitt's Lymphoma (BL), wherein the animal model is a transgenic non-human animal that expresses the following transgenes: (a) a MYC transgene, wherein the MYC transgene comprises a nucleic acid sequence encoding MYC coupled to an inducible expression control sequence, wherein B cell-specific expression of the MYC transgene can be selectively repressed; (b) a transgene encoding a pre-rearranged B cell receptor (BCR) that is overexpressed in the B cell lineage of the animal and that selectively binds to an antigen; and (c) a transgene encoding a soluble form of the antigen bound by the BCR in (ii). Preferably, the transgene of (c) is expressed ubiquitously in the animal or is otherwise expressed so that it is available systemically (e.g., the antigen could be secreted, shed, etc.). In this embodiment of the invention, B cell-specific expression of the MYC transgene is not repressed in the animal (i.e., from birth forward, the overexpression of the MYC transgene in the animal is allowed to occur without inhibition).

In this embodiment, the MYC transgene comprises a nucleic acid sequence encoding MYC coupled to an inducible expression control sequence, wherein B cell-specific expression of the MYC transgene can be selectively repressed. For example, the MYC transgene can include a nucleic acid sequence encoding Myc coupled to a tetracycline (TET) responsive expression control element (TRE), the expression of which is controlled by a B-cell specific TET-off repressor, such as MMTV-rtTA. Such transgenes have been described above, and include, for example, TRE-MYC expressed in conjunction with MMTV-rtTA. Repression can be controlled for example by administration of a compound that regulates expression, such as tetracycline or doxycycline in the case of the MMTV-rtTA/TRE-MYC combination described herein.

In one aspect of this embodiment, the MYC transgene could instead comprises a nucleic acid sequence encoding MYC coupled to an Ig heavy chain enhancer. Such transgenes have been discussed above, and include, for example, Eμ-MYC.

In one aspect of this embodiment, the BCR transgene is expressed under the control of the endogenous immunoglobulin promoter. In one aspect, the BCR transgene selectively binds to hen egg lysozyme (HEL), such as the BCR^{HEL} transgene described herein.

In one aspect of this embodiment, the antigen is overexpressed in the animal as a soluble antigen, although provision of a membrane form of the antigen is also encompassed by the invention. In one preferred embodiment, the antigen is ubiquitously expressed in the animal or is otherwise expressed so that it is available systemically. In the case of a BCR that selectively binds to HEL as described above, transgene can encode a soluble HEL, such as the sHEL transgene described herein. The invention is not limited to this combination of BCR and antigen.

In another embodiment of the invention, a second non-human animal model of Burkitt's Lymphoma (BL) is provided, wherein the animal model is a transgenic non-human animal that expresses the following transgenes: (a) a MYC transgene that is overexpressed in the B cell lineage in the animal; (b) a transgene encoding a pre-rearranged VDJ region of an Ig heavy chain of a B cell receptor (BCR) that selectively binds to an antigen; (c) a transgene encoding a lambda light chain of a BCR that binds to the antigen of (b); and (d) a transgene encoding a soluble form of the antigen in (b) and (c), wherein the transgene is expressed ubiquitously in the animal.

In one aspect of this embodiment, the MYC transgene comprises a nucleic acid sequence encoding MYC coupled to an Ig heavy chain enhancer. Such transgenes have been discussed above, and include, for example, Eμ-MYC. In one aspect of this embodiment, the MYC transgene comprises a nucleic acid sequence encoding MYC coupled to an inducible expression control sequence, wherein B cell-specific expression of the MYC transgene can be selectively repressed. For example, the MYC transgene can include a nucleic acid sequence encoding Myc coupled to a tetracycline (TET) responsive expression control element (TRE), the expression of which is controlled by a B-cell specific TET-off repressor, such as MMTV-rtTA. Such transgenes have been described above, and include, for example, TRE-MYC expressed in conjunction with MMTV-rtTA. Repression can be controlled for example by administration of a compound that regulates expression, such as tetracycline or doxycycline in the case of the MMTV-rtTA/TRE-MYC combination described herein.

In one aspect of this embodiment, the BCR transgene is expressed under the control of the endogenous immunoglobulin promoter. In one aspect, the BCR transgene selectively binds to hen egg lysozyme (HEL), such as the BCR^{HEL} transgene described herein.

In one aspect of this embodiment, the BCR transgene is expressed under the control of the endogenous immunoglobulin promoter. In one aspect, the transgene encodes a pre-rearranged VDJ region of an Ig heavy chain of a B cell receptor (BCR) that selectively binds to an antigen, wherein the transgene is designed to be knocked in to the Ig heavy chain locus of the animal. Such a transgene is exemplified by the VDJki transgene described herein. In addition to this knock-in, the animal expresses a transgene encoding a lambda light chain of the BCR that binds to the antigen of (b), exemplified by the transgene denoted Lt-tg and described herein.

In one aspect of this embodiment, the antigen is overexpressed in the animal as a soluble antigen, although provision of a membrane form of the antigen is also encompassed by the invention. In one preferred embodiment, the antigen is ubiquitously expressed in the animal. In the case of a BCR that selectively binds to HEL as described above, transgene can encode a soluble HEL, such as the sHEL transgene described herein. The invention is not limited to this combination of BCR and antigen.

In yet another embodiment of the invention, a third non-human animal model of Burkitt's Lymphoma (BL) is provided, wherein the animal model is a transgenic non-human animal that expresses the following transgenes: (a) a MYC transgene that is overexpressed in the B cell lineage in the animal; and (b) a transgene encoding a pre-rearranged B cell receptor (BCR) that is overexpressed in the B cell lineage of the animal and that binds to an endogenous self-antigen expressed by the animal.

In one aspect of this embodiment, the MYC transgene comprises a nucleic acid sequence encoding MYC coupled to an Ig heavy chain enhancer. Such transgenes have been discussed above, and include, for example, Eμ-MYC. In one aspect of this embodiment, the MYC transgene comprises a nucleic acid sequence encoding MYC coupled to an inducible expression control sequence, wherein B cell-specific expression of the MYC transgene can be selectively repressed. For example, the MYC transgene can include a nucleic acid sequence encoding Myc coupled to a tetracycline (TET) responsive expression control element (TRE), the expression of which is controlled by a B-cell specific TET-off repressor, such as MMTV-rtTA. Such transgenes have been described above, and include, for example, TRE-MYC expressed in conjunction with MMTV-rtTA. Repression can be controlled for example by administration of a compound that regulates expression, such as tetracycline or doxycycline in the case of the MMTV-rtTA/TRE-MYC combination described herein.

In one aspect of this embodiment, the BCR transgene is expressed under the control of the endogenous immunoglobulin promoter. In one aspect, the BCR binds to arsenate and to an endogenous self-antigen in the animal, such as the BCR encoded by the ARS.A1 transgene described herein.

Also encompassed for use in the panels and methods of the invention is a fourth non-human animal model of Burkitt's Lymphoma (BL), wherein the animal model is a transgenic non-human animal model that expresses the following transgenes: (a) a MYC transgene that is overexpressed in the B cell lineage in the animal; (b) a transgene encoding a pre-rearranged B cell receptor (BCR) that is overexpressed in the B cell lineage of the animal; and (c) a transgene encoding a soluble form of the antigen bound by the BCR in (b). Preferably, the transgene in (c) is expressed ubiquitously in the animal. An animal within this embodiment, comprising the transgenes, Eμ-MYC, BCR^{HEL}, and sHEL (i.e., a Eμ-MYC/BCR^{HEL}/sHEL mouse) has been previously described in Refaeli et al., (2005), PNAS 102(11):4097-4102.

Mice according to any of these embodiments of the invention can be produced by producing mice expressing one or more of each transgene, followed by cross-breeding, as needed to place all of the transgenes in the same genetic background, and/or by obtaining transgenic mice expressing one or more transgenes (e.g., commercially or from another source), and cross-breeding as necessary to place all of the transgenes in the same genetic background. The invention encompasses the parental transgenic lines and progeny thereof that continue to express the requisite transgenes.

Mouse Model for Follicular-Like Lymphoma (FLL)

One embodiment of the invention relates to mouse models of MYC-driven, antigen dependent Follicular-like Lymphoma (FLL), as well as methods to produce such mouse models, and methods of using such mouse models. Prior to the present invention, there was a lack of a good mouse model for FLL for the further understanding of the molecular and genetic basis of the pathogenesis of the disease as well as the use of the model for as a preclinical tool for the development of novel therapies and identification of therapeutically relevant targets. The present invention provides a solution to this need in the art by providing a mouse model of FLL that resembles the human disease in terms of the flow cytometric profile of the tumors, the sites of anatomical presentation, the histopathology, the grade of the disease (aggressiveness of the tumor), the anatomical site of MYC overexpression and age of onset (see Table 6). The key aspect for this model is the overexpression of oncogenes in the context of a defined autoreactive B-cell background. The mouse model is ready for use in testing compounds of pharmaceutical or research interest. In one embodiment, human proteins of interest will be retrovirally overexpressed in FLL tumors in order to directly determine the efficacy of anti-human monoclonal antibodies for preclinical use.

The currently available models of FLL rely on the overexpression of Bcl-2 early in the B-cell compartment (VavP-Bcl-2) in order to recapitulate the translocation that juxtaposes the Bcl-2 ORF onto the IgH promoter/enhancer elements. Those mice give rise to B-cell tumors that resemble FLL, but occur after more than 14 months in a sporadic fashion and with a penetrance of 20% or less. Moreover, the current mouse models do not give rise to tumors that satisfy the criteria defined herein for the present inventors' FLL models. This mouse model, and various other aspects of this embodiment of the invention are described Example 3.

Accordingly, one embodiment of the invention provides a non-human animal model of Follicular Like Lymphoma (FLL), comprising a transgenic non-human animal that expresses: (a) a MYC transgene, wherein the MYC transgene comprises a nucleic acid sequence encoding MYC coupled to an inducible expression control sequence, wherein B cell-specific expression of the MYC transgene can be selectively repressed; (b) a transgene encoding a pre-rearranged B cell receptor (BCR) that is overexpressed in the B cell lineage of the animal and that selectively binds to an antigen; and (c) a transgene encoding a soluble form of the antigen bound by the BCR in (b), wherein the transgene is expressed ubiquitously in the animal. In this embodiment, B cell-specific expression of the MYC transgene is repressed (or not induced) in the animal from the birth of the animal until the animal becomes an adult, followed by a lowered level of continued repression of the expression of the MYC transgene (or a low level of induction of the transgene), to induce FLL in the animal as defined in Example 3.

In this embodiment, the MYC transgene comprises a nucleic acid sequence encoding MYC coupled to an inducible expression control sequence, wherein B cell-specific expression of the MYC transgene can be selectively repressed. For example, the MYC transgene can include a nucleic acid sequence encoding Myc coupled to a tetracycline (TET) responsive expression control element (TRE), the expression of which is controlled by a B-cell specific TET-off repressor, such as MMTV-rtTA. Such transgenes have been described above, and include, for example, TRE-MYC expressed in conjunction with MMTV-rtTA. Repression can be controlled for example by administration of a compound that regulates expression, such as tetracycline or doxycycline in the case of the MMTV-rtTA/TRE-MYC combination described herein.

In one aspect of this embodiment, the MYC transgene could alternatively comprise a nucleic acid sequence encoding MYC coupled to an Ig heavy chain enhancer. Such transgenes have been discussed above, and include, for example, Eμ-MYC.

In one aspect of this embodiment, the BCR transgene is expressed under the control of the endogenous immunoglobulin promoter. In one aspect, the BCR transgene selectively binds to hen egg lysozyme (HEL), such as the BCR^{HEL} transgene described herein.

In one aspect of this embodiment, the BCR transgene is expressed under the control of the endogenous immunoglobulin promoter. In one aspect, the transgene encodes a pre-rearranged VDJ region of an Ig heavy chain of a B cell receptor (BCR) that selectively binds to an antigen, wherein the transgene is designed to be knocked in to the Ig heavy chain locus of the animal. Such a transgene is exemplified by the VDJki transgene described herein. In addition to this knock-in, the animal expresses a transgene encoding a lambda light chain of the BCR that binds to the antigen of (b), exemplified by the transgene denoted Lt-tg and described herein.

In one aspect of this embodiment, the antigen is overexpressed in the animal as a soluble antigen, although provision of a membrane form of the antigen is also encompassed by the invention. In one preferred embodiment, the antigen is ubiquitously expressed in the animal. In the case of a BCR that selectively binds to HEL as described above, transgene can encode a soluble HEL, such as the sHEL transgene described herein. The invention is not limited to this combination of BCR and antigen.

Another embodiment of the invention relates to the provision of a non-human animal model of Follicular Like Lymphoma (FLL), comprising a transgenic non-human animal that expresses: (a) a MYC transgene, wherein the MYC transgene comprises a nucleic acid sequence encoding MYC coupled to an inducible expression control sequence, wherein B cell-specific expression of the MYC transgene can be selectively repressed; and (b) a transgene encoding a pre-rearranged B cell receptor (BCR) that is overexpressed in the B cell lineage of the animal and that binds to an endogenous self-antigen expressed by the animal. As in the embodiment above, B cell-specific expression of the MYC transgene is repressed (or not induced) in the animal from the birth of the animal until the animal becomes an adult, followed by a lowered level of continued repression of the expression of the MYC transgene (or low level of induction), to induce FLL in the animal as defined in Example 3.

In one aspect of this embodiment, the MYC transgene comprises a nucleic acid sequence encoding MYC coupled to an Ig heavy chain enhancer. Such transgenes have been discussed above, and include, for example, Eμ-MYC. In one aspect of this embodiment, the MYC transgene comprises a nucleic acid sequence encoding MYC coupled to an inducible expression control sequence, wherein B cell-specific expression of the MYC transgene can be selectively repressed. For example, the MYC transgene can include a nucleic acid sequence encoding Myc coupled to a tetracycline (TET) responsive expression control element (TRE), the expression of which is controlled by a B-cell specific TET-off repressor, such as MMTV-rtTA. Such transgenes have been described above, and include, for example, TRE-MYC expressed in conjunction with MMTV-rtTA. Repression can be controlled for example by administration of a compound that regulates expression, such as tetracycline or doxycycline in the case of the MMTV-rtTA/TRE-MYC combination described herein.

In one aspect of this embodiment, the MYC transgene could alternatively comprise a nucleic acid sequence encoding MYC coupled to an Ig heavy chain enhancer. Such transgenes have been discussed above, and include, for example, Eμ-MYC.

In one aspect of this embodiment, the BCR transgene is expressed under the control of the endogenous immunoglobulin promoter. In one aspect, the BCR binds to arsenate and to an endogenous self-antigen in the animal, such as the BCR encoded by the ARS.A1 transgene described herein.

Mice according to this embodiment of the invention can be produced by producing mice expressing one or more of each transgene, followed by cross-breeding, as needed to place all of the transgenes in the same genetic background, and/or by obtaining transgenic mice expressing one or more transgenes (e.g., commercially or from another source), and cross-breeding as necessary to place all of the transgenes in the same genetic background. The invention encompasses the parental transgenic lines and progeny thereof that continue to express the requisite transgenes.

Mouse Model for Diffuse-Like B-Cell Lymphoma (DLBCL)

One embodiment of the present invention relates to a mouse model of MYC-driven, antigen dependent Diffuse-like B-cell Lymphoma (DLBCL), as well as methods to produce such mouse models, and methods of using such mouse models. Prior to the present invention, there was a lack of a good mouse model for DLBCL for the further understanding of the molecular and genetic basis of the pathogenesis of the disease, as well as the use of the model for as a preclinical tool for the development of novel therapies and identification of therapeutically relevant targets. The present invention provides a solution to this need in the art by providing a mouse model of DLBCL that resembles the human disease in terms of the flow cytometric profile of the tumors, the sites of anatomical presentation, the histopathology, the grade of the disease (aggressiveness of the tumor), the anatomical site of MYC overexpression and age of onset (see Table 8). The key aspect for this model is the overexpression of oncogenes in the context of a defined autoreactive B-cell background. The mouse model is ready for use in testing compounds of pharmaceutical or research interest. In one embodiment, human proteins of interest will be retrovirally overexpressed in DLBCL tumors in order to directly determine the efficacy of anti-human monoclonal antibodies for preclinical use.

The currently available models of DLBCL rely on the overexpression of Bcl-6 in Germinal Center B-cells (Eμ-Bcl-6) in order to recapitulate the translocation that juxtaposes the Bcl-6 ORF onto the IgH promoter/enhancer elements. Those mice give rise to B-cell tumors that resemble DLBCL, but occur after more than 14 months in a sporadic fashion and with a penetrance of 20% or less. Moreover, the current mouse models do not give rise to tumors that satisfy the criteria defined herein for the present inventors' DLBCL models. This mouse model, and various other aspects of this embodiment of the invention are described Example 4.

Accordingly, one embodiment of the invention provides a non-human animal model of Diffuse Large B Cell Lymphoma (DLBCL), comprising a transgenic non-human animal that expresses: (a) a MYC transgene, wherein the MYC transgene comprises a nucleic acid sequence encoding MYC coupled to an inducible expression control sequence, wherein B cell-specific expression of the MYC transgene can be selectively repressed; (b) a transgene encoding a pre-rearranged B cell receptor (BCR) that is overexpressed in the B cell lineage of the animal and that selectively binds to an antigen; and (c) a transgene encoding a soluble form of the antigen bound by the BCR in (b), wherein the transgene is expressed ubiquitously in the animal. In this embodiment, the B cell-specific expression of the MYC transgene in the animal is repressed (or not induced) from the birth of the animal until the animal becomes an adult. At this point, the repression of the expression of the MYC transgene is completely ceased in the animal (or induction continuously applied), to induce DLBCL in the animal, such as described in Example 4.

In this embodiment, the MYC transgene comprises a nucleic acid sequence encoding MYC coupled to an inducible expression control sequence, wherein B cell-specific expression of the MYC transgene can be selectively repressed. For example, the MYC transgene can include a nucleic acid sequence encoding Myc coupled to a tetracycline (TET) responsive expression control element (TRE), the expression of which is controlled by a B-cell specific TET-off repressor, such as MMTV-rtTA. Such transgenes have been described above, and include, for example, TRE-MYC expressed in conjunction with MMTV-rtTA. Repression can be controlled for example by administration of a compound that regulates expression, such as tetracycline or doxycycline in the case of the MMTV-rtTA/TRE-MYC combination described herein.

In one aspect of this embodiment, the MYC transgene could alternatively comprise a nucleic acid sequence encoding MYC coupled to an Ig heavy chain enhancer. Such transgenes have been discussed above, and include, for example, Eμ-MYC.

In one aspect of this embodiment, the BCR transgene is expressed under the control of the endogenous immunoglobulin promoter. In one aspect, the BCR transgene selectively binds to hen egg lysozyme (HEL), such as the BCR^{HEL} transgene described herein.

In one aspect of this embodiment, the BCR transgene is expressed under the control of the endogenous immunoglobulin promoter. In one aspect, the transgene encodes a pre-rearranged VDJ region of an Ig heavy chain of a B cell receptor (BCR) that selectively binds to an antigen, wherein the transgene is designed to be knocked in to the Ig heavy chain locus of the animal. Such a transgene is exemplified by the VDJki transgene described herein. In addition to this knock-in, the animal expresses a transgene encoding a lambda light chain of the BCR that binds to the antigen of (b), exemplified by the transgene denoted Lt-tg and described herein.

In one aspect of this embodiment, the antigen is overexpressed in the animal as a soluble antigen, although provision of a membrane form of the antigen is also encompassed by the invention. In one preferred embodiment, the antigen is ubiquitously expressed in the animal. In the case of a BCR that selectively binds to HEL as described above, transgene can encode a soluble HEL, such as the sHEL transgene described herein. The invention is not limited to this combination of BCR and antigen.

Another embodiment of the invention provides a non-human animal model of Diffuse Large B Cell Lymphoma (DLBCL), comprising a transgenic non-human animal that expresses: (a) a MYC transgene, wherein the MYC transgene comprises a nucleic acid sequence encoding MYC coupled to an inducible expression control sequence, wherein B cell-specific expression of the MYC transgene can be selectively repressed; and (b) a transgene encoding a pre-rearranged B cell receptor (BCR) that is overexpressed in the B cell lineage of the animal and that binds to an endogenous self-antigen expressed by the animal. As in the embodiment above, the B cell-specific expression of the MYC transgene in the animal is repressed (or not induced) from the birth of the animal until the animal becomes an adult. At this point, the repression of the expression of the MYC transgene is completely ceased in the animal (or induction continuously applied), to induce DLBCL in the animal, such as described in Example 4.

In this embodiment, the MYC transgene comprises a nucleic acid sequence encoding MYC coupled to an inducible expression control sequence, wherein B cell-specific expression of the MYC transgene can be selectively repressed. For example, the MYC transgene can include a nucleic acid sequence encoding Myc coupled to a tetracycline (TET) responsive expression control element (TRE), the expression of which is controlled by a B-cell specific TET-off repressor, such as MMTV-rtTA. Such transgenes have been described above, and include, for example, TRE-MYC expressed in conjunction with MMTV-rtTA. Repression can be controlled for example by administration of a compound that regulates expression, such as tetracycline or doxycycline in the case of the MMTV-rtTA/TRE-MYC combination described herein.

In one aspect of this embodiment, the MYC transgene could alternatively comprise a nucleic acid sequence encoding MYC coupled to an Ig heavy chain enhancer. Such transgenes have been discussed above, and include, for example, Eμ-MYC.

In one aspect of this embodiment, the BCR transgene is expressed under the control of the endogenous immunoglobulin promoter. In one aspect, the BCR transgene selectively binds to hen egg lysozyme (HEL), such as the BCR^{HEL} transgene described herein.

In one aspect of this embodiment, the antigen is overexpressed in the animal as a soluble antigen, although provision of a membrane form of the antigen is also encompassed by the invention. In one preferred embodiment, the antigen is ubiquitously expressed in the animal. In the case of a BCR that selectively binds to HEL as described above, transgene can encode a soluble HEL, such as the sHEL transgene described herein. The invention is not limited to this combination of BCR and antigen.

Mice according to this embodiment of the invention can be produced by producing mice expressing one or more of each transgene, followed by cross-breeding, as needed to place all of the transgenes in the same genetic background, and/or by obtaining transgenic mice expressing one or more transgenes (e.g., commercially or from another source), and cross-breeding as necessary to place all of the transgenes in the same genetic background. The invention encompasses the parental transgenic lines and progeny thereof that continue to express the requisite transgenes.

Methods to Produce Transgenic Animals of the Invention

Methods to produce the non-human animal models of the present invention include any methods for genetically modifying an animal that are known in the art, including methods of expressing a transgene in an animal and/or for otherwise modifying the genome of an animal, such as by knocking out or knocking in a particular gene, portion thereof, or regulatory region thereof. According to the present invention, a “genetically modified” animal, such as any of the preferred non-human animals described herein, has a genome which is modified (i.e., mutated or changed) from its normal (i.e., wild-type or naturally occurring) form such that the desired result is achieved (e.g., development of a phenotype consistent with a model of NHL as described herein). Genetic modification of an animal is typically accomplished using molecular genetic and cellular techniques, including manipulation of embryonic cells and DNA. Such techniques are generally disclosed for mice, for example, in “Manipulating the Mouse Embryo” (Andras Nagy, Marina Gertsenstein, Cold Spring Harbor Laboratory Press, 2003, incorporated herein by reference in its entirety), “Gene Targeting: A Practical Approach” (Edited by Alexandra A. Joyner, Oxford University Press, 2000), “Mouse Genetics and Transgenics: A Practical Approach” (Ian J. Jackson and Catherine A. Abbott, editors, Oxford University Press, 2000), “The Laboratory Mouse Handbook of Experimental Animals”, Hans Hedrich, editor, Elselvier Academic Press, 2004), “Transgenic Animals” (F. Grosveld and G. Kollias, editors, Academic Press; 1st edition, 1992), or “Transgenic Animal Technology: a Laboratory Handbook (2nd Ed.)” (Carl A. Pinkert, Lavoisier, 2002), each of which is incorporated by reference in its entirety. Additionally, techniques for genetic modification of a mouse through molecular technology are described in detail in the Examples section.

The genetically modified non-human animal models of the present invention can be characterized by several phenotypes which result from the genetic modifications described herein. The phenotypes of interest herein are those related to the development of a Non-Hodgkin's Lymphoma (NHL)-like disease, and particularly, a phenotype related to a form of NHL selected from B cell chronic lymphocytic leukemia/lymphoma (B-CLL), Burkitt's lymphoma (BL), Follicular-like lymphoma (FLL) and Diffuse large B cell lymphoma (DLBCL). Such phenotypic characteristics include: development of B cell tumors (B cell lymphoma), relevant latency to onset, aggressiveness of the lymphoma, anatomical presentation and evolution of the lymphoma, histopathology of the lymphoma, immunophenotype, and MYC overexpression. The specific phenotypes associated with the various forms of NHL are described in the Examples, along with a comparison of the phenotypes of the genetically modified animals to the phenotypes present in the corresponding human diseases.

According to the present invention, any non-human animal suitable for the development of an NHL-like disease as described herein and for use in the methods described herein may be used as a starting organism for the preparation of a transgenic or otherwise genetically modified non-human animal of the present invention. Preferably, the non-human model of the present invention is a mammal including, but not limited to, pigs, rabbits, primates and rodents. Most preferably, a transgenic model of the present invention is a rodent, and even more preferably, a mouse.

The preparation and uses of the non-human animal model of the invention will be described below with particular reference to a transgenic mouse. However, the transgenes and methods and uses for the transgenic mice of the present invention, as described herein in detail, can be modified and applied to any suitable mammal for the study of NHL and related conditions or for any method described herein, including other rodents. The invention is not limited to mice.

According to the present invention, a transgenic mouse is a mouse which includes a recombinant nucleic acid molecule (i.e., transgene) that has been introduced into the genome of the mouse at the embryonic stage of the mouse's development. As such, the transgene will be present in all of the germ cells and somatic cells of the mouse. Methods for the introduction of a transgene into a mouse embryo are known in the art and are described in detail in the technical references described above. For example, a recombinant nucleic acid molecule (i.e., transgene) can be injected into the pronucleus of a fertilized mouse egg to cause one or more copies of the recombinant nucleic acid molecule to be retained in the cells of the developing mouse. A mouse retaining the transgene, also called a “founder” mouse, usually transmits the transgene through the germ line to the next generation of mice, establishing transgenic lines. According to the present invention, a transgenic mouse also includes all progeny of a transgenic mouse that inherit the transgene, an egg that contains the transgene(s) and is capable of maturing into the mouse, as well as any parts of the animal (cells, tissues, organs, bodily fluids) that contain or are impacted by the presence of the transgene(s).

Techniques for achieving targeted integration of a nucleic acid molecule of interest into a genome are well known in the art and are described in the technical manuals for animal manipulation cited above. For example, the isolated nucleic acid molecule can be engineered into a targeting vector which is designed to integrate into a host genome. According to the present invention, a targeting vector is defined as a nucleic acid molecule which has the following features: (1) genomic sequence from the target locus in the host genome to stimulate homologous recombination at that locus; (2) a desired genetic modification within the genomic sequence from the target locus sufficient to obtain the desired phenotype; and (3) a selectable marker. Such targeting vectors are well known in the art. Following introduction of the isolated nucleic acid molecule of the targeting vector into the ES cells, ES cells which homologously integrate the isolated nucleic acid molecule are injected into mouse blastocysts and chimeric mice are produced. These mice are then bred onto the desired mouse background to detect those which transmit the mutated gene through the germ line. Heterozygous offspring of germline transmitting lines can then be mated to produce homozygous progeny.

According to the present invention, a transgene-negative littermate is a mouse which is born into the same litter as a transgenic mouse described herein (i.e., a littermate), but does not inherit the transgene (i.e., is transgene-negative). Such a mouse is essentially a normal, or wild-type, mouse and is useful as an age-matched control for the methods described herein. Additionally, a wild-type control can be an animal with the same genetic background as the background of the animal models described herein, without the genetic modifications described herein, or without all of the genetic modifications described herein (i.e., not necessarily littermates).

Transgenes and other nucleic acid molecules useful in the present invention have been described above. In addition to the DNA of interest, the transgene is typically constructed to include at least one expression control sequence, such as a promoter, or other expression control sequences necessary or desirable for proper expression and/or processing of the transgene or other nucleic acid molecule. These expression control sequences are operatively linked to the DNA of interest. The phrase “operatively linked” refers to linking of nucleic acid sequences in the transgene in a manner such that the transgene can be expressed in target cells (B cells) when the transgene is integrated into a host genome. The additional expression control sequences are well known in the art and include sequences which control the initiation, elongation, and termination of transcription (such as enhancer sequences and polyadenylation sequences).

Transgene sequences are cloned using a standard cloning system, and the transgene products are excised from the cloning vector, purified, and injected into the pronuclei of fertilized mouse eggs. Stable integration of the transgene into the genome of the transgenic embryos allows permanent transgenic mouse lines to be established.

Mouse strains which are suitable for the derivation of transgenic mice as described herein are any common laboratory mouse strain. Preferred mouse strains to use for the derivation of transgenic mice founders of the present invention are described in the Examples, but the invention is not limited to these strains. Preferably, founder mice are bred onto wild-type mice to create lines of transgenic mice.

Cell Lines of the Invention

The present invention also includes B cells isolated from any of the non-human animal models described herein, as well as B cell lines established from the isolated B cells, and B cell tumors isolated from the animals.

According to the present invention, general reference to “B cells” or “B lymphocytes” includes pre-B cells, splenic B cells, lymph node B cells, myeloma cells, lymphoma cells, peripheral blood B cells, bone marrow B cells and hybridoma cells. Hybridoma cells refer to hybrid cell lines comprising myeloma cells (tumor cells capable of being maintained in tissue culture but do not produce immunoglobulin) fused with, for example, a spleen cell capable of producing an immunoglobulin molecule. Reference to a “B cell antigen receptor” or “BCR” is intended to reference the B cell antigen receptor, which includes a membrane immunoglobulin (mIg) antigen binding component, or a biologically active portion thereof (i.e., a portion capable of binding a ligand and/or capable of associating with a transducer component), and transducer Ig-α and Ig-β components, or biologically active portions thereof (i.e., a portion capable of transducing an intracellular signal and/or capable of associating with an extracellular ligand binding portion).

Cells that are cultured directly from a subject are known as primary cells. With the exception of some derived from tumors, most primary cell cultures have limited lifespan. After a certain number of population doublings, cells undergo the process of senescence and stop dividing, while generally retaining viability. An established or immortalized cell line has acquired the ability to proliferate indefinitely either through random mutation, deliberate modification (e.g., retroviral transduction, hybridization, etc.), and/or provision of particular growth requirements sufficient to establish long term cultures. Methods of producing cells lines, including B cell lines, from primary B cells or B cell tumors are well known in the art. The Examples provide examples of the establishment and use of cell lines according to the present invention.

The present invention also includes panels of cells and cell lines isolated from, produced from or derived from any one, two, three, four, five, six, seven, eight, or more of the non-human animal models of the invention. The panel of cells and cell lines may also include cells or cell lines isolated from, produced from, or derived from any other animal model of NHL, any animal model of a related disease or condition, or any animal model for an unrelated disease or condition, or any control animal (negative or positive). Such panels of cells or cell lines are useful in any of the methods described herein (any research, development, pharmaceutical, or clinical purpose, including but not limited to, the identification, development, and/or testing of drugs (therapeutics, prophylactics, etc.), targets, markers, and/or research tools for use in the diagnosis of, study of, and/or treatment of any Non-Hodgkin's Lymphoma, such as those described herein, or for any related condition). In particular, the panels of cells, since they may include cells or cell lines from one or more of the different forms of NHL described herein, can be extremely valuable in identifying drug candidates that are specific for a particular form or forms of NHL, and/or for differentiating the causes of, targets of, diagnosis of, and/or therapies for a particular form or forms of NHL.

Methods of Use Exemplifying the Invention

The mouse models of the present invention can be used for any research, development, pharmaceutical, or clinical purpose, including but not limited to, the identification, development, and/or testing of drugs (therapeutics, prophylactics, etc.), targets, markers, and/or research tools for use in the diagnosis of, study of, and/or treatment of any Non-Hodgkin's Lymphoma, such as those described herein, or for any related condition. Various uses of these mouse models will be apparent to those of skill in the art and all such methods are encompassed herein. Accordingly, other embodiments of the invention relate to methods to produce the mouse models of the invention, and to use the mouse models of the invention in any method for the identification, development, and/or testing of drugs, targets, markers, and/or research tools for use in the diagnosis of, study of, or treatment of any Non-Hodgkin's Lymphoma, or for any related condition. The methods can be practiced using any suitable technique associated with the use of the mouse models of the invention, or any tissue, cells, or organs thereof. The invention is not limited to in vivo use of the mouse models. Methods of production and use of genetically modified animals, including transgenic animals, are described in a variety of technical manuals, such as those cited previously herein.

In one aspect of the invention, the present inventors have developed a novel method in which a biological agent specific to a human protein that may be important in the biology of maintenance of large B-cell NHLs can be directly tested in the mouse models of the invention for preclinical purposes.

While there are no other validated models of NHLs that meet the criteria that the present inventors have established, the closest technology presently available is the use of xenotransplantation models in which human tumor cells or cell lines are transplanted into a NOD/SCID mouse. The disease that arises in these other models differs in many important ways from the lymphomas that develop in humans, and do not recapitulate the pathology. The results of preclinical testing of drug candidates in that setting has not been of predictive value for the human case, and since the results are not predictive, they have rarely been useful in human clinical trials. In contrast, the present invention provides validated models in which the biology of initiation and maintenance resembles the human disease. The introduction and/or replacement of target proteins with human proteins will allow for the direct testing of a drug candidates in a preclinical model that the present inventors believe will provide a more predictive platform of drug testing in vivo.

The mouse models of the present invention have been produced and are readily used to test compounds for pharmaceutical and research use. In one embodiment, the human proteins of interest can be overexpressed in the mouse tumors by any method (e.g., retroviral transduction) in order to directly determine the efficacy of anti-human monoclonal antibodies and other compounds for preclinical use. Other methods of expression of human proteins and biologically active portions thereof in these mouse models are encompassed by the invention and will be apparent to those of skill in the art.

This method of the invention, and various other aspects of this embodiment of the invention are described in Example 5.

Accordingly, one embodiment of the invention includes a method for preclinical testing of drug candidates for non-Hodgkin's Lymphoma (NHL). One aspect of the method includes administering to any one or more of the non-human animal models or panels of animal models described herein, a candidate drug for NHL, and detecting whether the candidate drug inhibits tumors in the animal model(s), wherein candidate drugs that inhibit tumors in the animal model, or that inhibit any other phenotype of NHL disease as described herein, are selected for clinical testing. Another aspect of this embodiment includes the contacting of candidate drugs to any one of more of the B cell lines described herein, and detecting whether the B cell line is sensitive to the candidate drug, wherein candidate drugs to which the B cell line(s) is sensitive is selected for clinical testing. As used herein, to be “sensitive” to a drug, means that in response to contact with the drug, the cell line experiences a genetic or biological change, which most typically results in a loss in viability of the cell line.

In one aspect of this embodiment, a candidate drug that inhibits tumors in a first mouse model but not in a second mouse model (or in a B cell line from a first mouse model but not a second mouse model) is selected for clinical testing as a specific inhibitor of the form of NHL exhibited by the first mouse model. As discussed above, the method of the invention is particularly useful for identifying candidate drugs that are useful for the treatment of one or more specific forms of NHL, while perhaps not be useful for the treatment of all forms. Such drugs may have advantages of decreased toxicity to the patient and/or increased specificity for the target lymphomas.

In another aspect of any of the above-identified embodiments, as described previously, the mouse model or cell can be further genetically modified to comprise a human nucleic acid molecule of interest or to express a human protein of interest (e.g., by the tumors in the mouse or by the B cell lines). In this embodiment, the nucleic acid molecule or protein is a target for human NHL, and the method can include a step of detecting whether the candidate drug changes the expression or biological activity of the target or the phenotype of the mouse or cell as compared to in the absence of the candidate drug. This method is particularly useful for preclinical screening of human candidate drugs that are predicted to have a beneficial effect in the prevention or treatment of an NHL or a symptom or phenotype associated with NHL.

Another embodiment of the invention relates to a method to identify a target for use in the diagnosis, study or treatment of a Non-Hodgkin's Lymphoma (NHL) or condition related thereto. In one aspect, the embodiment includes comparing the expression of genes by any one or more of the animal models described herein or by a panel of animals described herein, to the expression of genes by a control animal that does not have an NHL, and identifying genes that are differentially expressed in the NHL animal model, or the proteins encoded by the genes, as targets for use in the diagnosis, study or treatment of NHL. In another aspect, the embodiment includes comparing the expression of genes by one of more of any of the B cell lines described herein, or any panel of B cell lines described herein, to the expression of genes by a control B cell line from an animal that does not have an NHL, and identifying genes that are differentially expressed in the NHL animal model, or the proteins encoded by the genes, as targets for use in the diagnosis, study or treatment of NHL. As discussed above, the method of the invention is particularly useful for identifying candidate drugs that are useful for the treatment of one or more specific forms of NHL, while perhaps not be useful for the treatment of all forms. Such drugs may have advantages of decreased toxicity to the patient and/or increased specificity for the target lymphomas.

In a similar method to that described above, instead of detecting gene expression, the biological activity of a gene or protein in the animal model(s) or in the B cell line(s) is detected and compared to the appropriate control, in order to identify differences in biological activity in one or more of the mouse models as compared to the control animal, to identify targets for use in the diagnosis, study or treatment of NHL.

In one aspect of any of the above embodiments, the method includes a first step of contacting the animal or cells with a test compound, prior to the step of comparing. In this aspect of the invention, the effect of test compounds on the expression or biological activity of a target or animal or disease phenotype can be evaluated.

Embodiments of the invention as described above can include the evaluation of the molecular and biochemical events associated NHL. Such methods can include the steps of (a) harvesting cells, tissues or body fluids from a non-human animal model of the present invention; and, (b) comparing the cells, tissues or body fluids from the non-human animal model to cells, tissues or body fluids from a wild-type (non-NHL model) of the non-human animal. The step of harvesting may be performed using any of the well known methods of harvesting cells, tissues and/or body fluids from an animal, and depend on the tissues to be studied and the status of the experiment to be performed. For example, cells can be harvested by biopsy, dissection, or lavage; tissues can be harvested by surgery, biopsy or dissection; and body fluids can be harvested by withdrawal, swiping, or lavage.

The step of comparing is performed by an assay that is suitable for the tissue to be evaluated and the goal of the experiment. For example, suitable assays which might be performed on the cells, tissues, and/or body fluids of a non-human animal model of the present invention include, but are not limited to: morphological examination of the cells, tissues or body fluids; histological examination of the cells, tissues or body fluids; detection of gene and/or protein expression; and biological assays for B cell function. A variety of such assays are well known in the art.

Various methods of detection of changes in genotypic or phenotypic characteristics of cells in any of the assays of the invention are known in the art. Examples of methods that can be used to measure or detect gene sequence or expression include, but are not limited to, polymerase chain reaction (PCR), reverse transcriptase-PCR (RT-PCR), in situ PCR, quantitative PCR (q-PCR), in situ hybridization, Southern blot, Northern blot, sequence analysis, microarray analysis, detection of a reporter gene, or other DNA/RNA hybridization platforms. Methods to measure protein levels, include, but are not limited to: Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS), flow cytometry, and assays based on a property of the protein including but not limited to DNA binding, ligand binding, interaction with other protein partners, cell signal transduction, enzyme activity, and secretion of soluble factors or proteins.

In drug screening assays, the term “test compound”, “putative inhibitory compound”, “candidate compound or drug” or “putative regulatory compound” refers to compounds having an unknown or previously unappreciated regulatory activity in a particular process. As such, the term “identify” with regard to methods to identify compounds is intended to include all compounds, the usefulness of which as a compound for a particular purpose is determined by a method of the present invention, preferably in the presence and absence of such a compound. Compounds to be screened in the methods of the invention include known organic compounds such as antibodies, products of peptide libraries, and products of chemical combinatorial libraries. Compounds may also be identified using rational drug design. Such methods are known to those of skill in the art and can involve the use of three-dimensional imaging software programs. For example, various methods of drug design, useful to design or select mimetics or other therapeutic compounds useful in the present invention are disclosed in Maulik et al., 1997, Molecular Biotechnology: Therapeutic Applications and Strategies, Wiley-Liss, Inc., which is incorporated herein by reference in its entirety.

In any of the above-described assays that are conducted in vitro, the conditions under which a cell, cell lysate, nucleic acid molecule or protein of the present invention is exposed to or contacted with a putative regulatory compound, such as by mixing, are any suitable culture or assay conditions, which can include the use of an effective medium in which the cell can be cultured (e.g., as described above) or in which the cell lysate can be evaluated in the presence and absence of a putative regulatory compound. Cells of the present invention can be cultured in a variety of containers including, but not limited to, tissue culture flasks, test tubes, microtiter dishes, and petri plates. Culturing is carried out at a temperature, pH and carbon dioxide content appropriate for the cell. Such culturing conditions are also within the skill in the art, and particularly suitable conditions for culturing conditionally immortalized stem cells of the present invention are described in detail elsewhere herein. Cells are contacted with a putative regulatory compound under conditions which take into account the number of cells per container contacted, the concentration of putative regulatory compound(s) administered to a cell, the incubation time of the putative regulatory compound with the cell, and the concentration of compound administered to a cell. Determination of effective protocols can be accomplished by those skilled in the art based on variables such as the size of the container, the volume of liquid in the container, conditions known to be suitable for the culture of the particular cell type used in the assay, and the chemical composition of the putative regulatory compound (i.e., size, charge etc.) being tested.

With regard to in vivo assays described herein, it is useful, although not essential, to prepare formulations comprising an amount of at least one regulatory compound to be evaluated according to the present invention, either alone or in combination with a pharmaceutically acceptable salt and/or complexed with another suitable carrier (described below). Such formulations can be formulated for any route of administration, including, but not limited to, intravenous administration, intraperitoneal administration, intramuscular administration, intranodal administration, intracoronary administration, intraarterial administration (e.g., into a carotid artery), subcutaneous administration, transdermal delivery, intratracheal administration, subcutaneous administration, intraarticular administration, intraventricular administration, intraspinal, pulmonary administration, oral, intranasal, aerosol, impregnation of a catheter, and direct injection into a tissue. For example, formulations to be evaluated can be formulated in an excipient that the animal to be treated can tolerate. Examples of such excipients include water, saline, phosphate buffered solutions, Ringer's solution, dextrose solution, Hank's solution, polyethylene glycol-containing physiologically balanced salt solutions, and other aqueous physiologically balanced salt solutions. Nonaqueous vehicles, such as fixed oils, sesame oil, ethyl oleate, or triglycerides may also be used.

The formulations comprising one or more desired compounds typically contain from about 0.1% to 90% by weight of the active compound, preferably in a soluble form, and more generally from about 0.1% to 1.0%.

In one embodiment of the present invention, a pharmaceutically acceptable carrier can include additional compounds that increase the half-life of a formulation in the treated animal. Suitable carriers include, but are not limited to, polymeric controlled release vehicles, biodegradable implants, liposomes, bacteria, viruses, other cells, oils, esters, and glycols.

In one embodiment of the present invention, a formulation can include a controlled release composition that is capable of slowly releasing the formulation into an animal. As used herein, a controlled release composition comprises a regulatory compound to be evaluated as described herein in a controlled release vehicle. Suitable controlled release vehicles include, but are not limited to, biocompatible polymers, polymeric matrices, capsules, microcapsules, microparticles, bolus preparations, osmotic pumps, diffusion devices, liposomes, lipospheres, and transdermal delivery systems. Other controlled release compositions of the present invention include liquids that, upon administration to an animal, form a solid or a gel in situ. Preferred controlled release compositions are biodegradable (i.e., bioerodible).

Novel Target for the Prophilaxis and Treatment of B-Cell NHL

Another embodiment of the invention relates to the identification of the tec tyrosine kinase Syk (spleen tyrosine kinase) as a novel molecular target that is important for the prophilaxis and treatment of B-cell Non-Hodgkin's Lymphomas, including B-cell chronic lymphocytic leukemia, Burkitt's lymphomas, Follicular like lymphomas and Diffuse large B-cell lymphomas.

Current therapies for large B-cell lymphomas, and Non-Hodgkin's lymphoma type tumors in particular, rely on traditional cytotoxic chemotherapeutic agents such as cyclophosphamide. There are well-established treatments for Burkitt's lymphoma, FLL and DLBCL. However, these regiments are highly toxic, logistically demanding, and expensive. Their efficacy also varies greatly, with remission rates ranging from 85% in the best of circumstances to 10% in the worst, and they are only marginally available in many areas of Africa where Burkitt's lymphoma is endemic. Moreover, the duration of the remissions can be as low as one month. Accordingly, there are ample and good reasons to consider additional therapeutic modalities. The present invention provides a solution to this problem. Moreover, the present invention overcomes the very harsh side effects incurred by traditional chemotherapy, including significant mortality soon after the therapy is delivered, as a result of “tumor lysis” syndrome.

Specifically, the development of the present invention mouse models of B-CLL, Burkitt's lymphoma, FLL and DLBCL that are MYC-driven and dependent upon continuous antigenic stimulation has allowed the inventors to begin to determine the nature and identity of the signals that are activated by the B-cell antigen receptor and cooperate with MYC in B-cell neoplasia. This will be a novel and rich source of molecular targets with therapeutic potential for the prophylaxis and treatment of large B-cell NHLs.

The current treatment protocols for NHL are based on the use of various doses of Cyclophosphamide, vincristine, prednisone, and similar cytotoxic agents. These were not developed based on their impact on the biological basis of the disease, but rather on the collective experience of using such chemotherapeutic agents in different tumor types. In addition, monoclonal antibodies to CD20 have recently emerged as a popular treatment, but these can not discriminate between normal and transformed B-cells, hence eliminating all B-cells from the patient for upwards of one year. The long term efficacy of anti-CD20 antibody based therapy of lymphomas remains to be determined at this time.

The present inventors have now demonstrated the effectiveness of the present invention using genetic approaches and with some chemical inhibitors. The transition of those experiments into in vivo models and human cells can now be readily achieved. This is exemplified by the demonstration of Syk as a novel target for inhibition for the prevention and treatment of NHLs, including demonstration of the efficacy of inhibition of Syk at both the genetic and biochemical level.

This target of the invention, and various other aspects of this embodiment of the invention are described in Example 6.

One embodiment of the present invention relates to a method to inhibit a B cell Non-Hodgkin's Lymphoma (NHL), comprising inhibiting tec tyrosine kinase Syk expression or activity. In one aspect, the NHL is selected from the group consisting of: B-cell chronic lymphocytic leukemia, Burkitt's lymphoma, Follicular like lymphoma and Diffuse large B-cell lymphoma. The inhibition of Syk can be achieved using a variety of methods for inhibition of a gene or protein known in the art. Such methods include, but are not limited to, administering to an NHL tumor an shRNA that selectively binds to Syk and inhibits the expression of Syk, or a drug that inhibits the expression or activity of Syk.

Syk tyrosine kinase is a widely expressed nonreceptor protein tyrosine kinase. Syk contains two Src homology 2 domains and multiple autophosphorylation sites. In blood cells, it couples immunoreceptors to transduction pathways regulating phagocytosis, cell differentiation, proliferation, adhesion, and motility. In B cells, it controls immunoreceptor-mediated calcium mobilization and phospholipase C activity, Ras/mitogen-activated protein kinase and phosphatidylinositol 3′-kinase/Akt survival pathways. The nucleic acid sequence encoding Syk in mice has been identified and is represented herein by SEQ ID NO:5. SEQ ID NO:5 encodes a Syk protein having the amino acid sequence represented herein by SEQ ID NO:6. In humans, the nucleic acid sequence encoding Syk has also been identified and is represented herein by SEQ ID NO:7. SEQ ID NO:7 encodes a Syk protein having the amino acid sequence represented herein by SEQ ID NO:8.

Another embodiment of the invention includes a method of identifying a regulatory compound that inhibits the expression or activity of Syk and thereby prevents or treats at least one symptom or condition of NHL, including any form of NHL. The method generally includes the step of contacting a putative regulatory compound to Syk or a nucleic acid molecule encoding Syk, in any cell-based, non-cell-based, or in vivo assay (e.g., using any of the non-human animal models or cell lines described herein), and detecting compounds that inhibit the expression or any biological activity of Syk under these assay conditions. The inhibition of Syk should have a beneficial effect on NHL by preventing or treating at least one symptom of the disease (e.g., as measured by changes in B cell activation and proliferation, reduction in tumor burden, inhibition of tumor growth, increased survival of the individual with NHL).

In one aspect of the above-methods of inhibiting or identifying, the regulatory compound can be selected from: an aptamer, an siRNA molecule, an antisense nucleic acid molecule, a ribozyme, an antibody or antigen binding fragment thereof, a conformational antagonist, and a small molecule inhibitor.

Aptamers are short strands of synthetic nucleic acids (usually RNA but also DNA) selected from randomized combinatorial nucleic acid libraries by virtue of their ability to bind to a predetermined specific target molecule with high affinity and specificity. Aptamers assume a defined three-dimensional structure and are capable of discriminating between compounds with very small differences in structure.

RNA interference (RNAi) is an approach for gene inactivation via gene silencing, termed “RNA interference” (RNAi). See, for example, Fire et al., Nature 391: 806-811 (1998) and U.S. Pat. No. 6,506,559. RNA interference refers to an event which occurs when an RNA polynucleotide acts through endogenous cellular processes to specifically suppress the expression of a gene whose sequence corresponds to that of the RNA. The silencing of the target gene occurs upon the degradation of mRNA by double strand (ds) RNA by the host animal, sometimes through RNAase III Endonuclease digestion. The digestion results in molecules that are about 21 to 23 nucleotides (or bases) in length (or size) although molecular size may be as large as 30 bases. These short RNA species (short interfering RNA or siRNA) mediate the degradation of corresponding RNA messages and transcripts, possibly via an RNAi nuclease complex, called the RNA-induced silencing complex (RISC), which helps the small dsRNAs recognize complementary mRNAs through base-pairing interactions. A short hairpin RNA (shRNA) is a sequence of RNA that makes a tight hairpin turn that can be used to silence gene expression via RNA interference. Following the siRNA interaction with its substrate, the mRNA is targeted for degradation, perhaps by enzymes that are present in the RISC. This type of mechanism appears to be useful to the organisms in inhibiting viral infections, transposon jumping, and similar phenomena, and to regulate the expression of endogenous genes. RNAi activity has been so far documented in plants, insects, nematodes and vertebrates among other organisms. For general background information, see, for example, Schutz et al., Virology 344(1):151-7 (2006); Leonard et al., Gene Ther. 13(6):532-40 (2006); Colbere-Garapin et al., Microbes Infect. 7(4):767-75 (2005); Wall, Theriogenology 57(1):189-201 (2002); El-Bashir, et al., Nature 411: 494-498 (2001); Fire, A., et al. Science 391: 806-811 (1998); Gitlin et al., Nature 418: 430-434 (2002); Gitlin, et al., J. Virol. 79:1027-1035 (2005); Kahana, et al., J. Gen. Virol. 85, 3213-3217 (2004); Kronke et al., J. Virol. 78: 3436-3446 (2004); Leonard et al., J. Virol. 79:1645-1654 (2005); and Yokota, et al., EMBO Rep. 4: 602-608 (2003).

A ribozyme is an RNA segment that is able to perform biological catalysis (e.g., by breaking or forming covalent bonds). More specifically, ribozymes are antisense RNA molecules that function by binding to the target RNA moiety and inactivate it by cleaving the phosphodiester backbone at a specific cutting site. Such nucleic acid-based agents can be introduced into host cells or tissues and used to inhibit the expression and/or function of Syk proteins.

An antibody of the invention includes polyclonal and monoclonal antibodies, divalent and monovalent antibodies, bi- or multi-specific antibodies, serum containing such antibodies, antibodies that have been purified to varying degrees, and any functional equivalents of whole antibodies. Isolated antibodies of the present invention can include serum containing such antibodies, or antibodies that have been purified to varying degrees. Whole antibodies of the present invention can be polyclonal or monoclonal. Alternatively, functional equivalents of whole antibodies, such as antigen binding fragments in which one or more antibody domains are truncated or absent (e.g., Fv, Fab, Fab′, or F(ab)² fragments), as well as genetically-engineered antibodies or antigen binding fragments thereof, including single chain antibodies or antibodies that can bind to more than one epitope (e.g., bi-specific antibodies), or antibodies that can bind to one or more different antigens (e.g., bi- or multi-specific antibodies), may also be employed in the invention.

The invention also extends to non-antibody polypeptides, sometimes referred to as binding partners, that have been designed to bind specifically to a Syk protein of the invention. Examples of the design of such polypeptides, which possess a prescribed ligand specificity are given in Beste et al. (Proc. Natl. Acad. Sci. 96:1898-1903, 1999), incorporated herein by reference in its entirety.

As used herein, an anti-sense nucleic acid molecule is defined as an isolated nucleic acid molecule that reduces expression of a protein by hybridizing under high stringency conditions to a gene encoding the protein. Such a nucleic acid molecule is sufficiently similar to the gene encoding the protein that the molecule is capable of hybridizing under high stringency conditions to the coding or complementary strand of the gene or RNA encoding the natural protein.

Small molecule inhibitors, including conformational antagonists, can be produced using any of the methods described above with respect to putative regulatory compounds.

Methods of formulating and administering test compounds are described above and are well known in the art.

Inhibition of expression or activity of Syk and/or inhibition of NHL associated with Syk inhibition can be detected using any method known in the art or as described in the Examples. For example, methods that can be used to measure or detect gene sequence or expression include, but are not limited to, polymerase chain reaction (PCR), reverse transcriptase-PCR (RT-PCR), in situ PCR, quantitative PCR (q-PCR), in situ hybridization, Southern blot, Northern blot, sequence analysis, microarray analysis, detection of a reporter gene, or other DNA/RNA hybridization platforms. Methods to measure protein levels, include, but are not limited to: Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS), flow cytometry, and assays based on a property of the protein including but not limited to DNA binding, ligand binding, interaction with other protein partners, cell signal transduction, enzyme activity, and secretion of soluble factors or proteins. Methods to detect an effect on NHL according to the invention include, but are not limited to, detection of regulation of latency to onset, modulation of tumor aggressiveness, modulation of anatomical presentation and evolution, modulation of histopathology, modulation of immunophenotype, modulation of MYC overexpression, reduction in tumor burden, decreased tumor growth, or increased survival.

According to the present invention, the methods disclosed herein are suitable for use in a subject that is a member of the Vertebrate class, Mammalia, including, without limitation, primates, livestock and domestic pets (e.g., a companion animal). Most typically, a patient will be a human patient. According to the present invention, the terms “patient”, “individual” and “subject” can be used interchangeably, and do not necessarily refer to an animal or person who is ill or sick (i.e., the terms can reference a healthy individual or an individual who is not experiencing any symptoms of a disease or condition).

Compounds and agents identified by the methods of the present invention as being useful for the prevention and treatment of any form or forms of NHL are encompassed for use in methods of prevention or treatment and in formulations and compositions for such use.

The following examples are provided for the purpose of illustration and are not intended to limit the scope of the present invention.

### EXAMPLES

### Experimental Procedures for Examples 1-4

Transgenic Mice and Transplantation of Tumors

Mice carrying the Eμ-MYC transgene have been described in detail previously (Adams et al. (1985) Nature 318, 533-538), and were obtained from the Jackson Laboratory. These mice express MYC in a B-cell specific manner, beginning at the Pre/Pro-B cell stage. The TRE-MYC and MMTV-rtTA mice have been described previously (TRE-MYC is in Felsher and Bishop, (1999) Mol. Cell 4, 199-207; MMTV-rtTA is in Hennighausen et al., (1995) J. Cell Biochem. 59, 463-472). The inventors crossbred these strains to combine the two transgenes in a novel single strain (MMTV-rtTA/TRE-MYC), in which the B-cell specific expression of the MYC transgene can be repressed by the administration of tetracycline or doxycycline. The inventors also utilized BCR^{HEL} mice, which express a pre-rearranged murine BCR from the endogenous immunoglobulin promoter (described in detail in Goodnow et al., (1988) Nature 334, 676-682) and sHEL mice, which ubiquitously express a transgene for the soluble form of soluble hen egg lysozyme under the control of the metallothionein promoter. This strain has also been described previously (Goodnow et al, 1988, ibid.) and were generously provided by Jason Cyster (University of California, San Francisco). All animals were maintained in accordance with the guidelines of the Committee on Animal Research at the University of California, San Francisco, National Jewish Medical and Research Center and the National Research Council.

All transgenic mouse lines were maintained on a C57/BL6 background, and were genotyped by PCR as previously described (Adams et al., 1985, supra; Goodnow et al., 1988, supra; Hartley et al., 1991). All animals were maintained in accordance with the guidelines of the Committee on Animal Research at the University of California, San Francisco and the National Research Council.

Adoptive transfers of cells and transplantation of tumors were done by injecting 10⁶ cells intravenously (unless otherwise indicated) into syngeneic (C57/BL6) females ranging in age from 4-6 weeks. For the experiments that involved tumor cells obtained from MMTV-rtTA/TRE-MYC/BCR^{HEL}/sHEL tumors, the recipient mice were sublethally irradiated (450 rads) in order to overcome some remaining allogeneic differences between the two strains.

Assessment of Tumorigenesis

The emergence of tumors was followed in three ways: i) physical examination of living animals and necropsy of deceased animals, particularly to detect enlargement of lymphoid organs and viscera; ii) counting the total number of cells in organs; and iii) the specific enumeration of B-cells carrying cell-surface receptor for the antigen HEL. Three pairs of lymph nodes were collected each time (two inguinal, two axillary, and two brachial lymph nodes). These lymph nodes were pooled and processed into single cell suspensions. Spleens and thymii were also collected and used to generate single cell suspensions. Each spleen or thymus was individually ground on a 60 μm wire mesh screen (Sigma, St. Louis, Mo.). The red blood cells were lysed in TAC buffer (0.017M Tris, pH 7.65 and 0.135M NH4Cl), as previously described (Hartley et al, 1991), and the resulting pellets were resuspended in complete lymphocyte media, which consists of RPMI1640+10% heat inactivated fetal calf serum, supplemented with L-glutamine, penicillin/streptomycin, nonessential amino acids, 2 mM HEPES, 2 mM sodium pyruvate and 10 mM β-mercaptoeathanol (all obtained from Invitrogen, Grand Island, N.Y.). Single cell suspensions were counted with a Coulter counter (Coulter Diagnostics, San Diego, Calif.). The percentage of viable cells was determined by uptake of 7-aminoactinomycin D (7AAD), and flow cytometry. The values for total cell numbers were used to derive the number of viable cells by multiplying percentage of viable cells (obtained from the 7AAD analysis) by the total number of cells (obtained from the Coulter counter analysis), and dividing by 100. These measurements were compared with microscopic counting of trypan-blue excluding cells in a hemocytometer.

In order to determine the number of B-cells carrying the BCR^{HEL} transgene, single cell suspensions were stained with antibodies to B220 and IgM^{a} (both obtained from Pharmingen laboratories, San Diego, Calif.), followed by flow cytometric analysis. The number of BCR^{HEL}+B-cells was determined by multiplying the percentage of B220⁺/IgM^{a+} cells (obtained from the FACS analysis) by the number of total viable cells and dividing by 100. These values were compared to stains performed using a pan-specific antibody to IgM (Pharmingen laboratories, San Diego, Calif.). This approach was used to determine the number of BCR^{HEL} expressing cells in all the cases where the mice were on a C57/BL6 background, where the allotype expressed is normally IgM^{b}. For the mice in which the genetic background was mixed (all the experiments that involved the MMTV-rtTA/TRE-MYC transgenes, see Examples 2-4 below), the number of BCR^{HEL} expressing cells was determined by HEL binding. Single cell suspensions were incubated with HEL (1 mg/ml, obtained from Sigma, St. Louis, Mo.) in FACS buffer. These cells were washed and incubated with Hy9-biotin, an HEL-specific monoclonal antibody (kindly provided by Dr. Jason Cyster, UCSF), followed by streptavidin-PE and B220-FITC (both obtained from Pharmingen laboratories, San Diego, Calif.).

Phenotypic Analysis of Cells

The surface phenotype of cells present in the lymphoid organs of normal and tumor bearing mice was analyzed by flow cytometry. Single cell suspensions were prepared from the lymph nodes, spleens, thymus and bone marrow. The cell suspensions were incubated with 1:50 dilutions of antibodies on ice for 30 minutes, and were then washed in FACS buffer (1% BSA in PBS+0.05% Sodium Azide) and fixed in PBS containing 1% paraformaldehyde. Cells were stained with antibodies to one or more of the following markers: B220, Thy1.2, Mac-1, IgM (pan), IgM^{a}, IgM^{b}, IgD (pan) and IgD^{a}, CD4, CD5, CD8, CD19, CD21, CD23, CD25, CD44, CD62L, CD69, CD80 and CD86 (all obtained from Pharmingen).

Molecular Analysis of Tumor Clonality

In order to determine the clonal composition of the tumors, the inventors adapted a protocol that has been described previously (Kline et al, 2001). Genomic DNA was extracted from 106 cells (from either spleen or lymph nodes) using the Quiagen genomic DNA mini-kit (Quiagen, Valencia, Calif.), following the manufacturer's specifications. 200 ng of genomic DNA was used for a nested PCR reaction. The first reaction consisted of 5 μl of 10×Taq buffer (Gibco/Invitrogen), 4 μl of 50 mM MgCl₂, 2.5 ng of VH-specific primer, 2.5 ng of JH specific primer, 2.5 nM dNTPs (Roche Diagnostics, Basel, Switzerland) and 2.5 U of Taq polymerase (Roche diagnostics, Basel, Switzerland) and distilled-deionized water to a final volume of 50 μl. The reactions were placed in a thermal cycler (MJ-Research, Watertown, Mass.) and subjected to a PCR cycle as previously described (Kline et al, 2001). A sample of 2 μl from the first reaction was used as a template for the second reaction of the nested PCR. This reaction was conducted as the first one, except that the primer pairs encoded sequences within the initial set used earlier. The sequences for all the primers used have been previously described (Kline et al, 2001). The PCR reaction products were fractionated in a 2% agarose/TAE gel, stained with ethidium bromide. Some of the PCR products were cloned using a TOPO-TA cloning kit (Invitrogen laboratories, San Diego, Calif.), following manufacturer's specifications, then sequenced using the Big Dye terminator cycle sequencing kit (Applied Biosystems, Redwood City, Calif.), following manufacturer's specification, at the UCSF GCRC core facility.

Antigen Stimulation in Vivo

Groups of 6 mice were injected intravenously with 100 μg of HEL protein in PBS. The nature of the responding cells was determined by flow cytometric analysis of single cell suspensions prepared from spleens and lymph nodes. Cells were stained with one of the following combinations of antibodies: B220 Cychrome C (CyC), Thy1.2 FITC, Mac-1 phycoerythrin (PE), in order to determine the proportion of the cells composed of B-cells, T-cells and myeloid cells, respectively; or B220 CyC, IgM^{a} PE, and CD86 FITC, in order to determine the number of BCR^{HEL} expressing B-cells that had upregulated B7-2 (CD86) expression; and B220 CyC, IgM^{a} PE, and CD69 FITC, in order to determine the number of BCR^{HEL} expressing B-cells that had upregulated CD69 expression on their surface.

Therapeutic Trials

Groups of 6 mice were utilized for each of the experimental protocols. Four mice bearing transplanted tumors, and two age and sex matched wild type mice were treated with the same drug and equal frequency. The transplant recipient mice were held until tumors became clinically apparent (approximately 100 days for the Eμ-MYC tumors, 58 days for the Eμ-MYC/BCR^{HEL} tumors, 21 days for the Eμ-MYC/BCR^{HEL}/sHEL tumors, and 14 days for the MMTV-rtTA/TRE-MYC/BCR^{HEL}/sHEL tumors). The mice then received daily injections of the indicated agents for 7 days, as applicable. Mice were either euthanized 24 hours after the last injection of drug, or held indefinitely to ascertain survival. Lymph nodes, spleens and bone marrows were collected and processed to generate single cell suspensions. The cells were counted as described above. An aliquot from the cell suspensions was stained with antibodies for B220, Thy 1.2, Mac-1, IgM^{a}, B7-2, and CD69, in order to determine the proportion of B-cells, T-cells and myeloid cells, as well as the activation status of the HEL-reactive B-cells. Treatments were performed with cyclosporin A (Bedford laboratories, Bedford Ohio) (2 mg/kg/day), FK506 (Prograf, Fujisawa Healthcare, Deerfield, Ill.) (2 mg/kg/day), rapamycin (Biomol, King of Prussia, Pa.) (2 mg/kg/day), and cyclophosphamide (1 mg/kg/day) (Bristol-Myers Squibb, Princeton, N.J.). The therapeutics were suspended in PBS, sterilized by filtration through a 0.22 μm membrane, and administered intravenously through the tail vein.

Tissue Processing and Histology

Normal and tumor tissues were fixed in 10% formallin and embedded in paraffin. Sections (4 μm) were stained with hematoxylin-eosin. Images were acquired with a CCD camera mounted on a phase-contrast microscope.

Serum ELISA

The levels of total serum immunoglobulins were determined using a capture ELISA, as previously described (Goodnow et al., (1991) Nature 352, 532-36). The levels of serum anti-HEL antibodies were determined by performing a solid phase ELISA for HEL, as previously described). Blood samples were obtained from mice and allowed to clot by incubating at room temperature for 2 hours. Samples were centrifuged for 30 minutes at 14000 rpm. Clear supernatants were collected and stored frozen until assayed. ELISAs were set up in triplicate wells. The sera were assayed in two different dilutions (1:300 and 1:900) for HEL reactivity. The sera were diluted starting at 1:100, 2 fold (up to 1:204800 dilution factor) for the IgM capture assays.

Tissue Processing and Immunofluorescence.

Tissues were fixed in 10% formalin and embedded in paraffin. Sections (4 μM) were stained with hematoxylin-eosin and evaluated for the presence of tumor cells and other abnormalities. Images were acquired with a CCD camera (Leica) mounted on a phase-contrast microscope.

For immunofluorescence, tissues were cyropreserved by embedding in O.C.T. compound (Tissue-Tek, Torrance, Calif.) and freezing in liquid Nitrogen. Sections (5 μM) were fixed in 4% Paraformaldehyde, blocked in 1% BSA in PBS, and stained with a Rhodamine-conjugated donkey anti-IgM (Biomeda, Foster City, Calif.), and mounted with DAPI-containing medium (Vector Laboratories, Burlingame, Calif.). Images were acquired with a Leica Camera mounted on a Fluorescence microscope.

### Example 1

The following example describes the production of a novel mouse model for the study of lymphomagenesis and for preclinical testing of therapeutics to treat chronic B-cell lymphocytic lymphomas (B-CLL) of humans.

Introduction of Antigenic Specificity into B-Cells Expressing a Transgene for MYC

In order to test the role of BCR signaling in lymphomagenesis, the inventors needed to generate mice containing B-cells that both overexpressed MYC and had a known antigenic specificity at a high frequency. To that end, a transgene for BCR^{HEL} was bred into Eμ-MYC mice, creating a novel strain designated Eμ-MYC/BCR^{HEL}, also referred to herein as a murine model for chronic B-cell lymphocytic lymphomas (B-CLL) of humans. Expression of the Eμ-MYC and BCR^{HEL} transgenes was targeted to the B-cell lineage (Adams et al, 1985; Goodnow et al, 1988). Mice that express the Eμ-MYC transgene alone appear developmentally normal at first (Langdon et al, 1986), but later accumulate a large number of Pro-B cells (B220+, CD43+, IgM−, IgD−) in their bone marrow, and eventually also in their peripheral lymphoid organs (Adams et al, 1985). In contrast, the bone marrow and lymph nodes of Eμ-MYC/BCR^{HEL} mice of the invention contained normal numbers of mature B-cells, which expressed BCR^{HEL} on their surface (data not shown). Thus, the developmental arrest normally observed in Eμ-MYC mice was apparently corrected by the introduction of an antigen receptor transgene, in accord with previous results (Chang et al, 1995). The Eμ-MYC/BCR^{HEL} mice provided a means to test for cooperation between signaling from the BCR and overexpression of MYC in the genesis of lymphoid tumors.

Expression of BCR^{HEL} in the B-Cell Lineage Altered Lymphomagenesis by MYC

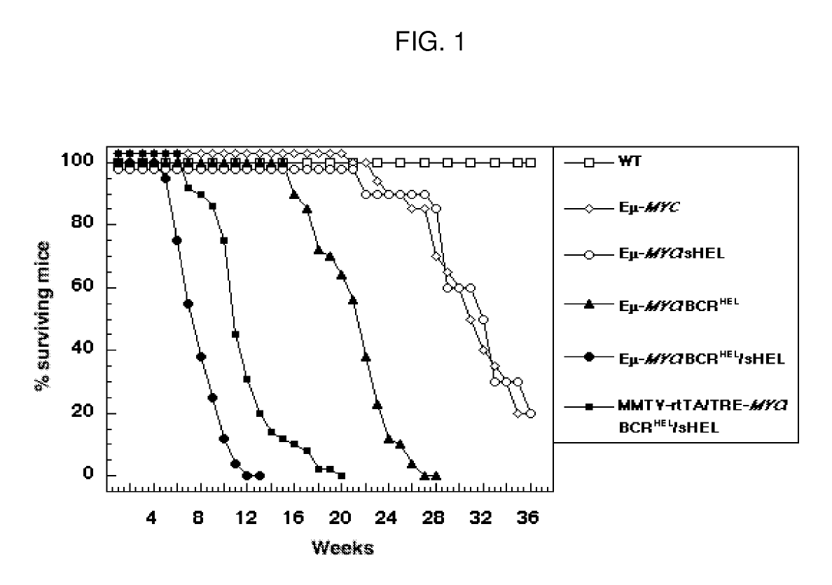

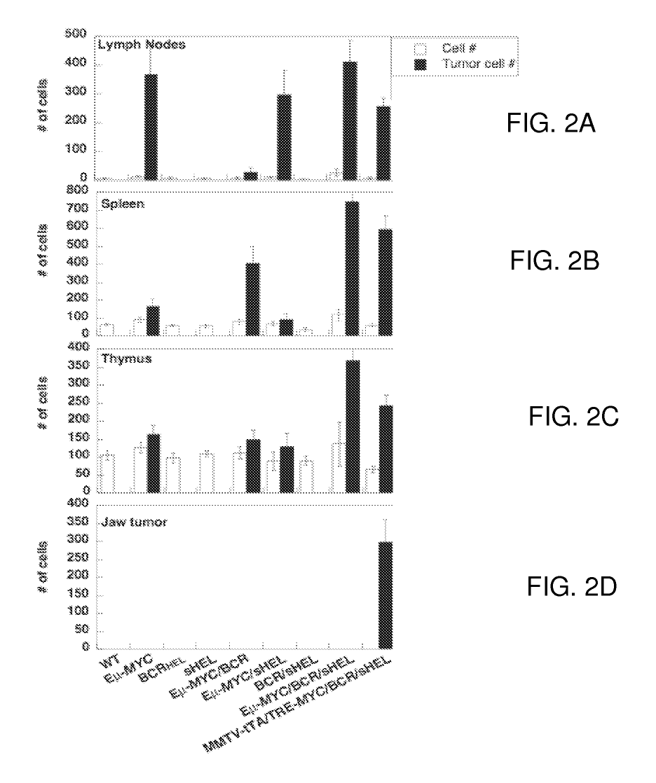

The Eμ-MYC/BCR^{HEL} mice developed fatal lymphomas more rapidly than did E-MYC mice (FIG. 1), and the anatomical distribution of the tumor was different (FIGS. 2A-2D). The emergence of tumors was followed in three ways: by anatomical inspection; by counting the total number of cells in organs (FIGS. 2A-2D); and by flow cytometric analysis to enumerate B-cells carrying BCR^{HEL} (data not shown). Referring to FIG. 1, strains of mice in groups of 50 were observed over a period of 36 weeks. Deceased mice were examined by necropsy. Death was uniformly attributable to lymphoid tumors.

Referring to FIGS. 2A-2D, healthy animals were euthanized at 21 days of age, tumorous Eμ-MYC mice at 200-240 days, Eμ-MYC/BCR^{HEL} mice at 112-130 days, Eμ-MYC/BCR^{HEL}/sHEL mice at 26-30 days, and MMTV-rtTA/TRE-MYC/sHEL/BCR^{HEL} mice at 71-86 days. All tumors contained homogeneous populations of cells with distinctive surface phenotypes: B220+/IgM− cells for Eμ-MYC tumors, B220+/IgM^{a+} cells for both Eμ-MYC/BCR^{HEL} and Eμ-MYC/BCR^{HEL}/sHEL tumors, and B220+/BCR^{HEL} cells for MMTV-rtTA/TRE-MYC/BCR^{HEL}/sHEL tumors.

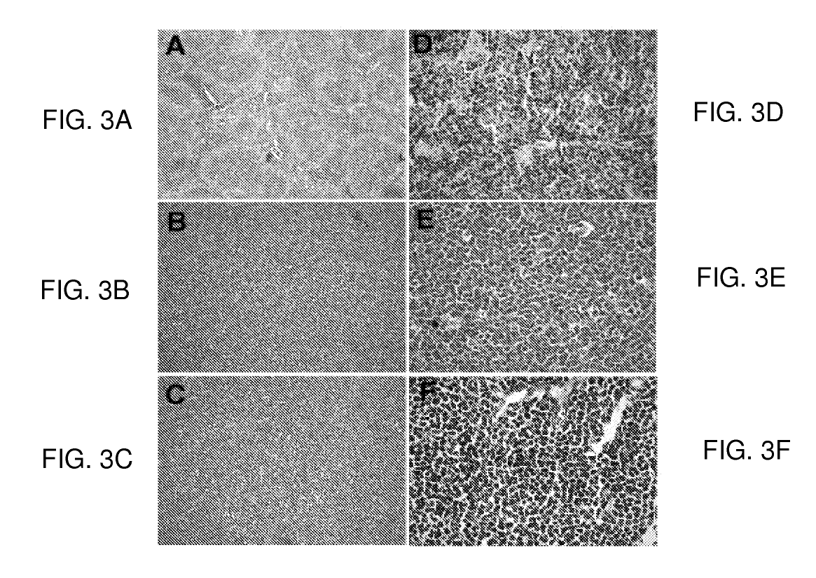

Evidence of tumor in Eμ-MYC/BCR^{HEL} mice appeared first in the spleen at about 18 weeks of age, then in lymph nodes and the bone marrow. Histological examination of the tumors revealed a diffuse and homogeneous population of small lymphocytes (FIG. 3C, 3F), in contrast to the more heterogeneous composition of the Eμ-MYC tumors (FIG. 3B, 3E). Analysis with a panel of markers identified the tumor cells as mature but naïve B-cells (Table 1), whereas the Eμ-MYC tumors were composed of Pre/Pro B-cells (Table 1, in accordance with Adams et al, 1985, supra).

TABLE 1
Cell surface markers of B-cell lymphomas¹

| | | | | MMTV- | | | |
|---|---|---|---|---|---|---|---|
| | | | Eμ- | rtTA/ | | | |
| | | Eμ- | MYC/ | TRE-MYC/ | Human | | |
| Cell Surface | Eμ- | MYC/ | BCR^{HEL}/ | BCR^{HEL}/ | Burkitt | Human | Human |
| Marker | MYC | BCR^{HEL} | sHEL | sHEL | lymphoma | B-CLL | DLBCL |
| B220 | + | + | + | + | + | + | + |
| CD19 | + | + | + | + | + | + | + |
| CD5 | + | − | − | − | − | + | − |
| CD21 | − | + | + | + | + | + | + |
| CD23 | − | + | − | − | − | + | − |
| PNA | − | − | + | + | ND² | − | ND² |
| B7-2 | − | − | + | + | + | − | + |
| CD69 | − | − | + | + | ND² | − | ND² |
| IgM | − | + | + | + | + | + | + |
| BCR^{HEL3} | − | + | + | + | NA⁴ | NA⁴ | NA⁴ |
| ¹Single cell suspensions were generated from lymph nodes, spleens, or bone marrow from mice with the indicated genotypes, stained with the indicated markers, and analyzed by flow cytometry. All stains were compared to healthy wild type mice, and the tumor bearing mice were harvested at a time consistent with the onset of disease presented in FIG. 1. The expression of a cell-surface marker is defined as positive (+) when the mean fluorescence in measurements with | | | | | | | |
| # flow cytometry is higher than | | | | | | | |

²Not determined
³The levels of BCR^{HEL} protein present on the surface of these cells was determined as a result of HEL-protein binding. The cells were incubated with HEL (1 mg/ml) and subsequently with an anti-HEL antibody (Hy9).
⁴Not applicable. This antigenic specificity was introduced experimentally into the mouse models. It is not a feature of human BL or B-CLL or DLBCL.

The Response of Tumors to Immunosuppression

To determine if this murine model of lymphomas would be useful for the testing of novel anti-cancer therapeutics, the inventors tested the effect of several immunosuppressants on existing tumors. Cyclosporin A, FK506, and rapamycin were injected to treat well advanced tumors that had been initiated by transplantation. The effects of these agents were compared to that of cyclophosphamide, an agent commonly used to treat human BL (Schiffer, 2001).

10⁶ cells obtained from tumor-bearing spleen or lymph nodes were transplanted into cohorts of 4-10 mice. The recipient mice were held for observation until they developed externally obvious lymphadenopathy (approximately 100 days for the Eμ-MYC tumors, 58 days for the Eμ-MYC/BCR^{HEL} tumors. The tumor bearing and control wild-type mice were then treated daily for seven days with intravenous injections of the various drugs. Mice were either euthanized 24 hours after the last injection of drug, or held indefinitely to ascertain survival. The analysis of tumor burden was performed with cells obtained from lymph nodes and spleens.

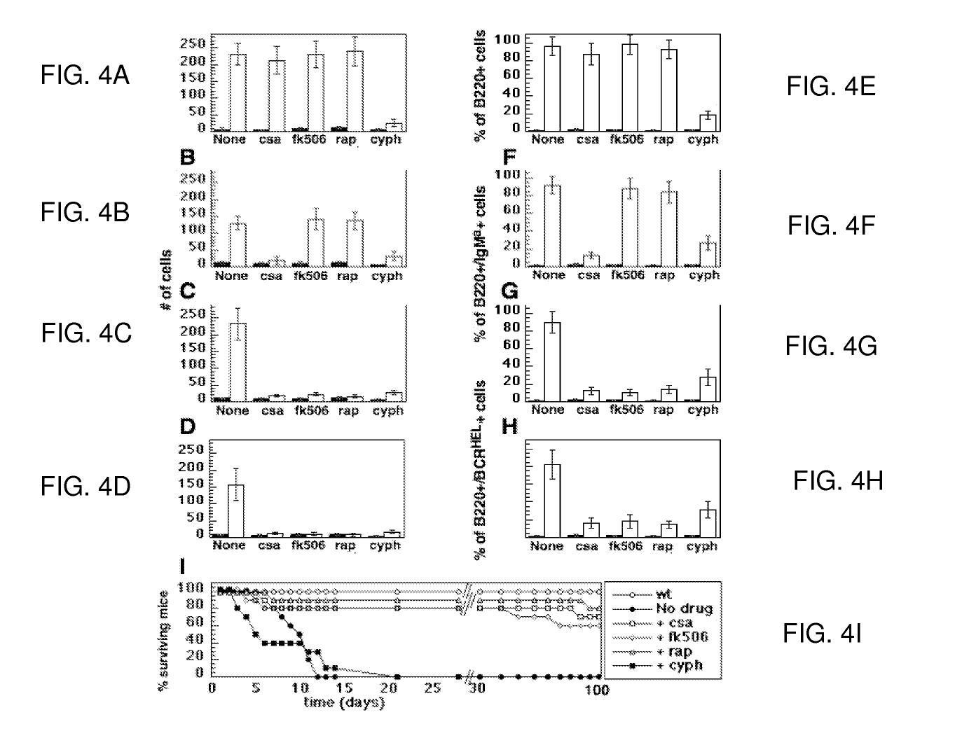

The Eμ-MYC tumors did not respond to any of the immunosuppressive drugs tested (FIGS. 4A and 4E). Disease progressed at the same rate in treated and untreated mice. Histological examination of the affected organs also revealed no evidence of therapeutic response (data not shown). However, the transplanted Eμ-MYC tumors showed a strong response to cyclophosphamide, as previously described (Schmitt et al, 2002). Treatment with cyclophosphamide elicited tumor regression in all animals, but also caused a more general cytotoxicity, manifested as a reduction in T-cells, myeloid cells, and non-transgenic B-cells (data not shown). Similar toxicity from cyclophosphamide was also observed in wild type mice. The tumors derived from the Eμ-MYC/BCR^{HEL} mice responded to cyclosporin and cyclophosphamide, but not to either FK506 or Rapamycin (FIG. 4B, 4F).

The Tumor in Eμ-MYC/BCR^{HEL} Mice Resembles Chronic B-Cell Lymphocytic Lymphomas of Humans.

The tumors in Eμ-MYC/BCR^{HEL} and MMTV-rtTA/TRE-MYC/BCR^{HEL} mice resemble B-CLL in humans (Table 2). The similarities include relative indolence of the disease, anatomical presentation, histopathology and surface phenotype. But the similarity is not complete. In particular, abnormal expression of MYC has not been implicated in the genesis of indolent B-CLL (Caligaris-Cappio and Hamblin, 1999), whereas it plays a vital role in the murine model. Instead, occasional progression of human B-CLL to a more aggressive disease is sometimes accompanied by overexpression of MYC (Caligaris-Cappio and Hamblin, 1999; Rosenwald et al, 2001; Klein et al, 2001). In addition, the inventors have shown that the tumors that arose in Eμ-MYC/BCR^{HEL} bigenic mice were oligoclonal, as opposed to the mostly monoclonal tumors that arise in Eμ-MYC mice. This is likely a result of having two oncogenic drivers in the instance of B-CLL, further corroborating the notion that a BCR is able to provide a transforming signal that cooperates with overexpressed MYC, in the absence of its cognate ligand (Table 3).

TABLE 2
Comparison of human and mouse tumors

| | | | Anatomical | | | |
|---|---|---|---|---|---|---|
| | Latency | | presentation | | | |
| | to | | and | | | MYC |
| | onset¹ | Aggressiveness | evolution | Histopathology | Immunophenotype | overexpression |
| B-CLL | 61 | Indolent, until | Spleen, | Small | Mature, | Yes, 8; 14 and |
| | years of | histological | subsequent | lymphocytes | Naïve, | 8; 22 |
| | age | transformation | spreading to | with clear | CD5+ B- | translocations |
| | | | lymph nodes, | chromatin and | cells | |
| | | | bone | scant cytoplasm | | |
| | | | marrow, liver, | | | |
| | | | lungs² | | | |
| Eμ- | 18 | Longer latency | Spleen, | Small | Mature, | Yes, |
| MYC/BCR^{HEL} | weeks | than two other | subsequent | lymphocytes | Naïve, | Eμ-MYC |
| transgenic | | mouse models | spreading to | with clear | CD5− B- | transgene |
| mice | | described here | lymph nodes, | chromatin and | cells | |
| | | | bone | scant | | |
| | | | marrow, liver, | cytoplasm³ | | |
| | | | lungs² | | | |

¹Mean age of highest incidence in humans (Grogan et al, 1982; Rowe et al, 1985). The latency for the murine tumors is defined as the median of the data in FIG. 1.
²Documentation of anatomical locations was determined by flow cytometric analysis for B220+/IgM− cells for Eμ-MYC tumors, B220+/IgM^{a}+ cells for Eμ-MYC/BCR^{HEL}.
³The tumors in the Eμ-MYC/BCR^{HEL} mice consisted of a homogeneous population of lymphoid cells that appeared to be centrocytes.

TABLE 3
The clonality of tumors.

| Mouse | Individual Clones¹ | Germ line configuration |
|---|---|---|
| C57/BL-6 (wild type) | TMTC² | + |
| C57/BL-6 (lpr/lpr) | TMTC² | + |
| Eμ-MYC | 1-2 | 0 |
| Eμ-MYC/BCR^{HEL} | 10-15 | 0 |

¹Individual clones were represented by clearly distinguishable bands that migrated on an agarose gel differently from the germ line configurations, and appeared only when a specific combination of primers for a V_{H} and a J_{H} segment was used for the PCR reaction (data not shown).
²Too many to count. This refers to polyclonal populations, which yielded a smear in an agarose gel, as opposed to clearly distinguishable individual bands.

### Example 2

The following example describes the production of a novel mouse model for the study of lymphomagenesis and for preclinical testing of therapeutics to treat human Burkitt's lymphoma (BL).

Antigenic Stimulation Altered Tumorigenesis by MYC

In order to explore how antigen stimulation of BCR^{HEL} might affect tumorigenesis by MYC, the inventors bred a ubiquitously expressed transgene for sHEL into the Eμ-MYC/BCR^{HEL} background. The resulting strain (Eμ-MYC/BCR^{HEL}/sHEL) developed tumors even more rapidly than did Eμ-MYC/BCR^{HEL} mice (FIG. 1).

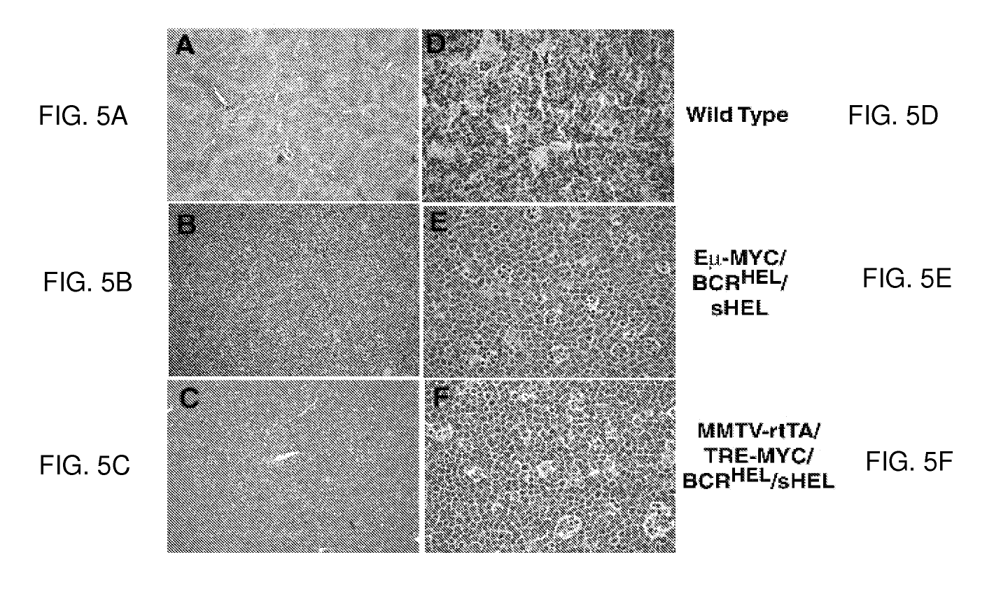

Overgrowth of B-cells could be detected in the bone marrow, lymph nodes, spleen and thymus (FIGS. 2A-2D). B-cells also infiltrated the liver, lungs, and central nervous system. Compression and invasion of the spinal cord caused paralysis of the hind and fore limbs. Histological examination revealed a homogeneous population of large lymphocytes in the spleen, lymph nodes, thymus and bone marrow. The sheets of cells had a “starry sky” appearance (FIGS. 5B, 5C, 5E and 5F) that is common among large B-cell lymphomas and is a prominent feature of BL (Morse et al, 2002). When examined for surface markers, the tumor cells closely resembled mature, activated, “post-germinal center” B-lymphocytes (Table 1 (see Example 1)), again reminiscent of BL. As expected, the tumor cells were specific for HEL, as evidenced by their ability to bind the antigen (Table 1, see Example 1).

The inventors also bred the BCR^{HEL} and sHEL transgenes into a second strain of mice that expresses MYC in the B-cell lineage (MMTV-rtTA/TRE-MYC). The final composite strain was designated MMTV-rtTA/TRE-MYC/BCR^{HEL}/sHEL. The inventors originally created these mice for other purposes, but the manner in which they developed tumors proved noteworthy for the present context. The mice died somewhat later than the Eμ-MYC/BCR^{HEL}/sHEL mice, but earlier than the other strains analyzed in the present study (FIG. 1). In a striking departure from the inventors' previous experience, however, tumors appeared first in the jaw, in a randomly unilateral manner (data not shown). The mice eventually developed a more generalized disease, with tumor cells appearing in multiple lymphoid organs and infiltrating non-lymphoid tissues as well (FIG. 2 and data not shown). The histological appearance of the MMTV-rtTA/TRE-MYC/BCR^{HEL}/sHEL tumors (FIGS. 5C and 5F) was similar to that of the Eμ-MYC/BCR^{HEL}/sHEL tumors (FIGS. 5B and 5E), including a starry sky appearance. The surface phenotype of MMTV-rtTA/TRE-MYC/BCR^{HEL}/sHEL tumor cells also resembled that of Eμ-MYC/BCR L/sHEL tumors (Table 1). The jaw tumors were covered with a thin layer of calcified material (data not shown), a feature not associated with tumors at other sites or in the other strains of mice.

In summary, the constitutive and antigen-stimulated forms of BCR^{HEL} altered tumorigenesis by MYC in distinctive manners. The distinctions involved diverse features of the tumors, including rate of appearance, anatomical presentation and progression, histopathology, and immunophenotype (Table 4). The tumors that arose in the presence of antigen stimulation resembled BL in multiple ways and were similar in two strains of mice with different MYC transgenes.

TABLE 4
Comparison of human and mouse tumors

| | Latency | | Anatomical | | | |
|---|---|---|---|---|---|---|
| | to | | presentation | | | MYC |
| | onset¹ | Aggressiveness | & evolution | Histopathology | Immunophenotype | overexpression |
| Burkitt | 4-6 | Very | Jaw, lymph | “Starry-sky”, | Mature, | Yes, 8; 14 and |
| Lymphoma | years of | aggressive | nodes, bone | large | activated B | 8; 22 |
| | age | | marrow, | lymphocytes | cells. | translocations |
| | | | central | with | | |
| | | | nervous | clumped | | |
| | | | system, | chromatin | | |
| | | | gastrointestinal | and clear | | |
| | | | tract | cytoplasm | | |
| Eμ- | 7 weeks | Very | Spleen, lymph | “Starry-sky”, | Mature, | Yes, |
| MYC/BCR^{HEL}/sHEL | | aggressive | nodes, bone | large | activated B | Eμ-MYC |
| transgenic mice | | | marrow, | lymphocytes | cells. | transgene |
| | | | thymus and | with | | |
| | | | central | clumped | | |
| | | | nervous | chromatin | | |
| | | | system² | and clear | | |
| | | | | cytoplasm³ | | |
| MMTV-rtTA/ | 10 | Very | Jaw, | “Starry-sky”, | Mature, | Yes, |
| TREMYC/BCR^{HEL}/ | weeks | Aggressive | subsequent | large | activated B | MMTV- |
| sHEL | | | spreading to | lymphocytes | cells. | rtTA/TRE-MYC |
| transgenic mice | | | lymph nodes, | with | | transgene |
| | | | spleen, bone | clumped | | |
| | | | marrow, liver, | chromatin | | |
| | | | lungs² | and clear | | |
| | | | | cytoplasm³ | | |

¹Mean age of highest incidence in humans (Grogan et al, 1982; Rowe et al, 1985). The latency for the murine tumors is defined as the median of the data in FIG. 1.
²Documentation of anatomical locations was determined by flow cytometric analysis for B220+/IgM− cells for Eμ-MYC tumors, B220+/IgM^{a}+ cells for Eμ-MYC/BCR^{HEL}/sHEL tumors and B220+/BCR^{HEL}+ cells for MMTV-rtTA/TRE-MYC/BCR^{HEL}/sHEL tumors. Anatomical distribution was confirmed by histological examination.
³The tumors in the Eμ-MYC mice consisted of a mixed population of small cleaved and non-cleaved lymphoid cells, as well as larger centrocytes.

TABLE 5
The clonality of tumors

| | Individual | Germ line |
|---|---|---|
| Mouse | Clones¹ | configuration |
| C57/BL-6 (wild type) | TMTC² | + |
| C57/BL-6 (lpr/lpr) | TMTC² | + |
| Eμ-MYC | 1-2 | 0 |
| Eμ-MYC/BCR^{HEL}/sHEL | 20-40 | 0 |
| MMTV-rtTA/TRE-MYC/BCr^{HEL}/sHEL (jaw) | 15-30 | 0 |

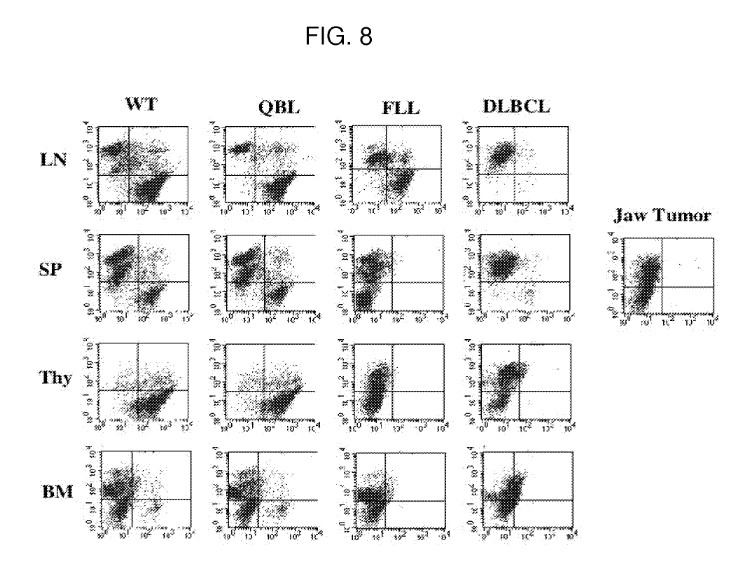

¹Individual clones were represented by clearly distinguishable bands that migrated on an agarose gel differently from the germ line configurations, and appeared only when a specific combination of primers for a V_{H} and a J_{H} segment was used for the PCR reaction (FIG. 8).
²Too many to count. This refers to polyclonal populations, which yielded a smear in an agarose gel, as opposed to clearly distinguishable individual bands (see FIG. 8).

The Response of Tumors to Immunosuppression

To determine if this murine model of lymphomas would be useful for the testing of novel anti-cancer therapeutics, the inventors tested the effect of several immunosuppressants on existing tumors. Cyclosporin A, FK506, and rapamycin were injected to treat well advanced tumors that had been initiated by transplantation. The effects of these agents were compared to that of cyclophosphamide, an agent commonly used to treat human BL (Schiffer, 2001).

10⁶ cells obtained from tumor-bearing spleen or lymph nodes were transplanted into cohorts of 4-10 mice. The recipient mice were held for observation until they developed externally obvious lymphadenopathy (approximately 100 days for the Eμ-MYC tumors, 21 days for the Eμ-MYC/BCR^{HEL}/sHEL tumors, and 14 days for the MMTV-rtTA/TRE-MYC/BCR^{HEL}/sHEL tumors). The tumor bearing and control wild-type mice were then treated daily for seven days with intravenous injections of the various drugs. Mice were either euthanized 24 hours after the last injection of drug, or held indefinitely to ascertain survival. The analysis of tumor burden was performed with cells obtained from lymph nodes and spleens.

The Eμ-MYC tumors did not respond to any of the immunosuppressive drugs tested (FIGS. 4A and 4E). Disease progressed at the same rate in treated and untreated mice. Histological examination of the affected organs also revealed no evidence of therapeutic response (data not shown). However, the transplanted Eμ-MYC tumors showed a strong response to cyclophosphamide, as previously described (Schmitt et al, 2002). Treatment with cyclophosphamide elicited tumor regression in all animals, but also caused a more general cytotoxicity, manifested as a reduction in T-cells, myeloid cells, and non-transgenic B-cells (data not shown). Similar toxicity from cyclophosphamide was also observed in wild type mice.

The tumors from Eμ-MYC/BCR^{HEL}/sHEL mice, as well as tumors from the jaws of MMTV-rtTA/TRE-MYC/BCR^{HEL}/sHEL mice, responded to both cyclophosphamide and all three of the immunosuppressants tested (FIGS. 4C, 4D, 4G, 4H).

Remissions of Eμ-MYC/BCR^{HEL}/sHEL tumors persisted for at least 5 months, following a seven-day course of treatment with immunosuppressants (FIG. 4I and data not shown). In contrast, the animals treated with cyclophosphamide entered a brief remission, but still died more rapidly than did untreated, tumor-bearing mice (FIG. 4I and data not shown), apparently consequent to the toxicity described above.

Establishment and Maintenance of Murine BL by Antigenic Stimulus and MYC Overexpression.

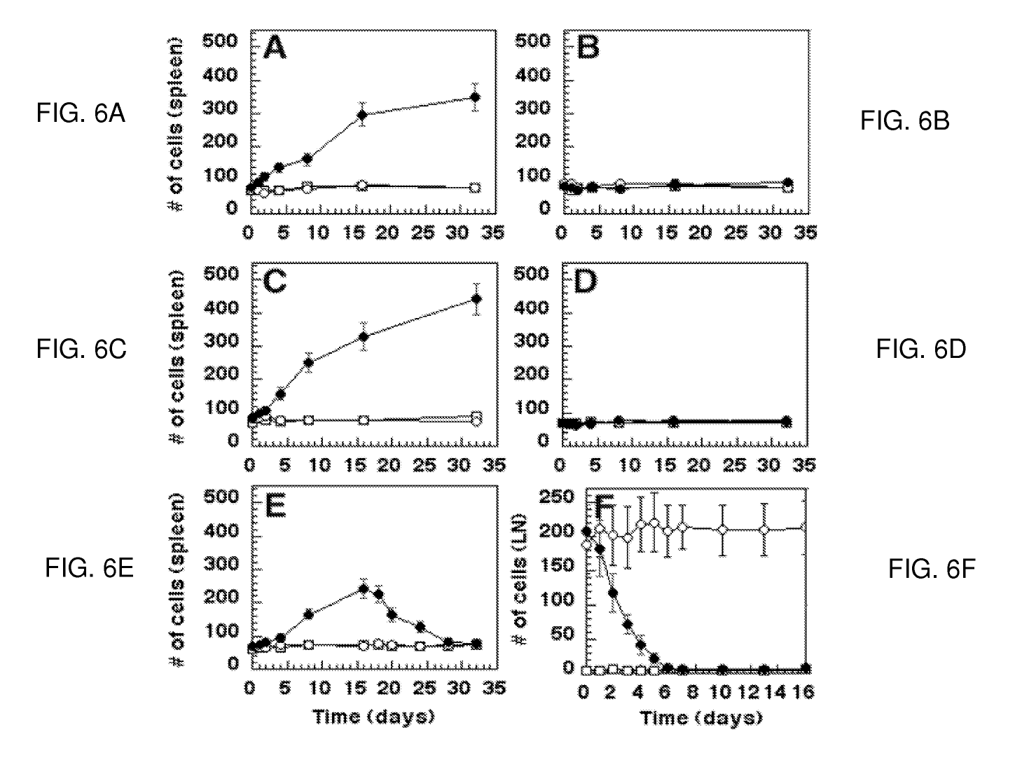

Referring to FIGS. 6A-6B (primary transplants), spleen and lymph node cells were harvested from an MMTV-rtTA/TRE-MYC/BCR^{HEL} mouse at 4 weeks of age. Cells from spleen and lymph nodes were pooled at a 1:1 ratio, and 10⁶ cells were introduced into either syngeneic wild type mice (empty circles) or sHEL transgenic mice (filled circles) by intravenous injection. Cohorts of mice were either kept on regular food (FIG. 6A), or on doxycycline-containing food (FIG. 6B). Tissues were collected at indicated time points from spleens and analyzed for total number of cells. Samples taken from wild type mice were analyzed at the same times (empty squares).

Referring to FIGS. 6C and 6D (secondary transplants), cells were collected from tumorous spleens and lymph nodes represented in FIG. 6A, 16 days after their initiation by transplantation. Cells from spleen and lymph nodes were pooled at a 1:1 ratio, and 10⁵ cells were introduced into either wild type recipients (empty circles) or sHEL transgenic mice (filled circles) by intravenous injection. The empty squares represent wild-type, unmanipulated mice that were analyzed in parallel with the experimental groups. Cohorts of mice were either kept on regular food (FIG. 6C), or on doxycycline-containing food (FIG. 6D). Cells were collected from spleens at the indicated times after the transplantation and analyzed as in FIGS. 6A and 6B.

Referring to FIGS. 6E and 6F, B-cell lymphomas regress after MYC overexpression is extinguished. FIG. 6E shows an experiment where a cohort of mice similar to those described in FIG. 6A was allowed to develop externally visible lymphadenopathy. 16 days later, those mice were switched to a doxycycline-containing diet. The empty circles represent wild type recipient mice that received transplants of MMTV-rtTA/TRE-MYC/BCR^{HEL} cells, the filled circles represent sHEL transgenic mice that received transplants of those cells, the empty squares represent wild-type, unmanipulated mice that were analyzed in these experiments in parallel with the experimental mice. Cells were collected from spleens at the indicated times after the transplantation and analyzed as in FIGS. 6A and 6B. FIG. 6F shows an experiment in which MMTV-rtTA/TRE-MYC/BCR^{HEL}/sHEL mice were allowed to develop tumors spontaneously, as a result of transgene function. Approximately 40 days later, mice with externally apparent lymphadenopathy were given doxycycline containing food (day 0 in figure). Cells were collected from lymph nodes at the indicated times after exposure of the mice to doxycycline and analyzed as in FIGS. 6A and 6B. The empty circles represent MMTV-rtTA/TRE-MYC/BCR^{HEL} mice that were never exposed to doxycycline, the filled circles represent MMTV-rtTA/TRE-MYC/BCR^{HEL} mice that were given doxycycline containing food after THEY developed externally apparent lymphadenopathy, the empty squares represent wild-type, unmanipulated mice that were analyzed in parallel with the experimental mice.

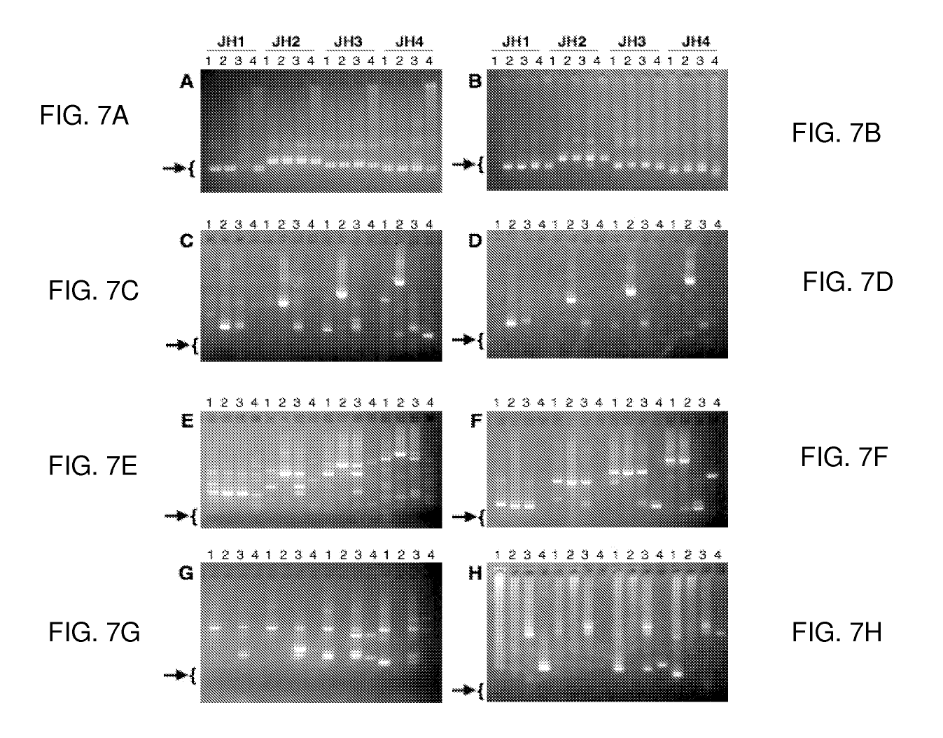

FIGS. 7A-7H are digital images showing the clonality of tumors in the mouse models of the invention. Genomic DNA was analyzed for VH to DJH rearrangements as described in Materials and Methods for these examples. The inventors examined rearrangements of sixteen different combinations of four V region genes (lanes numbered 1-4, as follows: 1 corresponds to 36-6, 2 to 81X, 3 to Q-52, and 4 to J558). These were tested in combination with the four JH genes listed in the figure (JH1-4). The arrows in the lower left corners of the panels indicate the PCR products that resulted from amplification of the germ line configuration. All of the rearranged VDJH products migrated more slowly in the gel. The data are representative of three different matched pairs of primary and transplanted tumors, for each tumor type. See also Table 5.

A Mouse Model for Burkitt Lymphoma

The experiments above describe two mouse models that develop a lymphoma with a close resemblance to human BL. The similarities include anatomical presentation and other clinical manifestations, histological appearance, and immunophenotype. A particularly striking finding was the unilateral occurrence of jaw tumors in the MMTV-rtTA/TRE-MYC/BCR^{HEL}/sHEL mice. This manifestation is characteristic of African BL (Burkitt, 1971), but remains unexplained in both the human and murine setting. Several previous reports have described experimental approaches that were aimed at developing mouse models of Burkitt lymphoma (Adams et al, 1985; Schmidt et al, 1988; Nussenzweig et al, 1988; Huang et al, 1995; Kovalchuk et al, 2000; Ruf et al, 2000). Only one of these attempts, however, produced a tumor with substantive resemblances to BL (Kovalchuk et al, 2000). That model used the control of the Igλ promoter and enhancer elements to express a mutant form of MYC that is found in human NHL. No provision was made for deliberate antigenic stimulation of B-cells, but the tumor cells did show evidence of immune selection, in the form of point mutations in the Ig loci. These findings prompted the authors to invoke antigenic stimulus in the genesis of the BL-like murine tumors as we have demonstrated directly.

BL appears in two major forms: endemic and sporadic. The endemic form is found mainly in Africa and is characterized by infection with Epstein Barr virus (EBV) (Rowe et al, 1985; Okano and Gross, 2001). In contrast, an association with EBV infection is found in only about 20% of sporadic BL, but chronic infection with another, as yet unidentified microbe might well figure in the remainder. Viral infection plays no role in the mouse models of BL described here. The inventors presume that the need for such infection has been circumvented by overexpression of the MYC transgene, which serves as a surrogate for the translocations that are a hallmark of human BL and are thought to occur subsequent to initiation of tumorigenesis by EBV or another agent (Hoffman et al, 2002).

With few exceptions, the tumor cells of BL have been described as monoclonal (Robinson et al, 1980; Magrath et al, 1983; Sklar et al, 1984; Magrath et al, 1989). In contrast, the tumor cells in the two animal models for BL described here are multiclonal. The human tumor presumably arises from a series of rare events, each amplified by clonal selection (Nowell, 1976; reviewed in Hanahan and Weinberg, 2000). The cumulative rarity in this sequence of events dictates that the eventual tumor is likely to be the product of a single clonal lineage. In contrast, the experimental model described here provides at least two potentially tumorigenic influences that are ubiquitous in the B-cell lineage of the transgenic animals: overexpression of MYC and stimulus by an autoantigen. Thus, a vast population of cells may be predisposed to tumor progression. Indeed, it is remarkable that the resulting tumors are composed of only a finite number of clones, suggesting the occurrence of clonal selection for tumorigenic events beyond those imposed experimentally. The results contrast sharply with the innumerable clones that proliferate to produce a relatively indolent disease in MRLlpr/lpr mice, a proliferation that is itself driven by autoimmunity (Watanabe-Fukunaga et al, 1992).

A variety of circumstantial evidence has implicated antigenic stimulus in the genesis of BL (Hecht and Aster, 2000). First, chronic infection with malaria in Africa is associated with an increased incidence of BL and accelerated progression of the disease (Kakufo and Burkitt, 1970; Facer and Khan, 1997). Second, the possibility of sustained antigenic stimulus is raised by the mature, activated immunophenotype characteristic of BL cells (Klein et al, 1995). Third, the sequences for the immunoglobulin molecules in many NHL, including BL, bear somatic mutations of the sort that normally arise during the process of affinity maturation (Chapman et al, 1995; Klein et al, 1995; Tamaru et al, 1995; Ottesmeier et al, 1998; Lossos et al, 2000; Harris et al, 2001; reviewed in Chapman et al, 1998). Whatever the role of antigenic stimulation in the genesis of BL, it would be cooperative with overexpression of MYC, which is a general feature of the tumor (Boxer and Dang, 2001). The present inventors' results with mouse models suggest that the hypothetical role of antigenic stimulus in the pathogenesis of BL should be investigated further.

### Example 3

The following example describes the production of a novel mouse model for the study of lymphomagenesis and for preclinical testing of therapeutics to treat human Follicular-like B-cell Lymphoma (FLL).

Antigenic Stimulation Altered Tumorigenesis by MYC

The inventors bred the BCR^{HEL} and sHEL transgenes into a strain of mice that expresses MYC in the B-cell lineage (MMTV-rtTA/TRE-MYC). This strain allows for the temporal and tissue specific overexpression of MYC in an autoreactive B-cell background. The final composite strain was designated MMTV-rtTA/TRE-MYC/BCR^{HEL}/sHEL. When the mice were maintained on a doxycycline-containing diet (200 mg/kg) (MYC transgenes remain silent) for four months, and then switched on to a diet containing a lower dose of doxycycline (50 mg/kg), the mice developed externally evident lymphadenopathy and splenomegaly accompanied by other clinical signs that are consistent with the onset of lymphoid neoplasia (scruffy fur, gasping, ascending hind limb paralysis, and dehydration). The tumors developed with an average latency of 9 weeks after the de-repression of the MYC transgene in those mice. While these tumors were more indolent than those obtained when we completely withdrew the mice from doxycycline (DLBCL-like), the FLL-like tumors proved to be universally lethal in these mice.

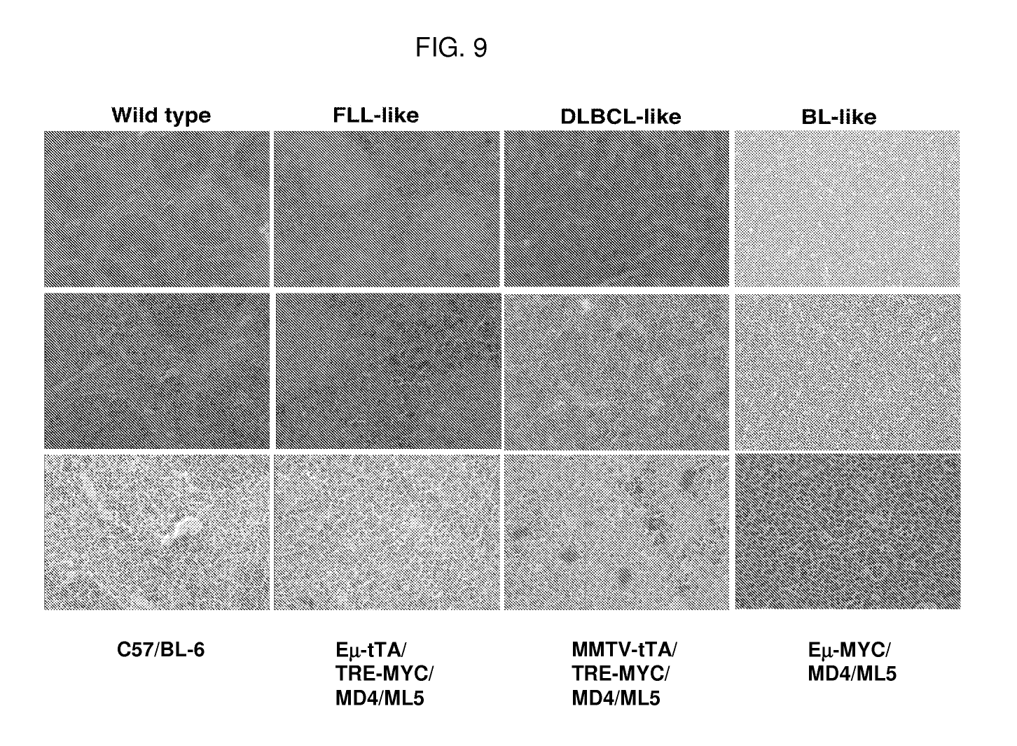

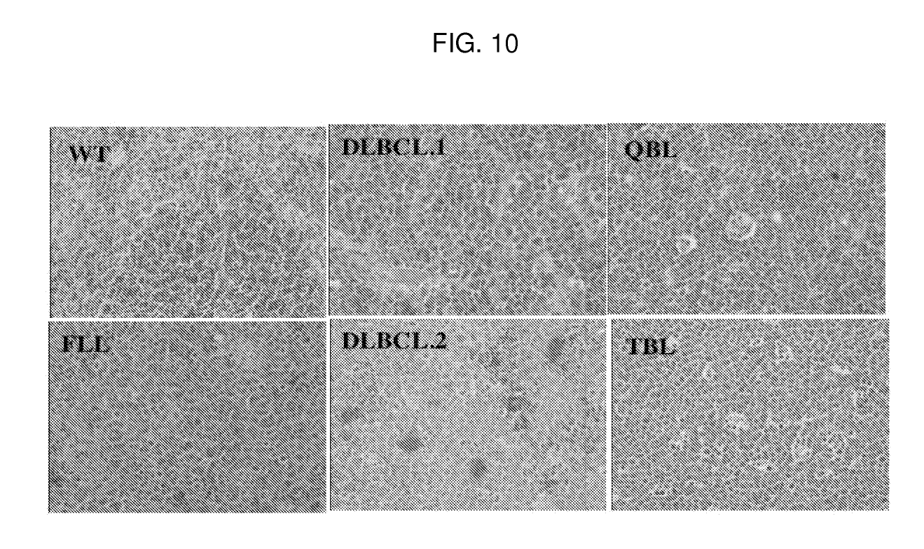

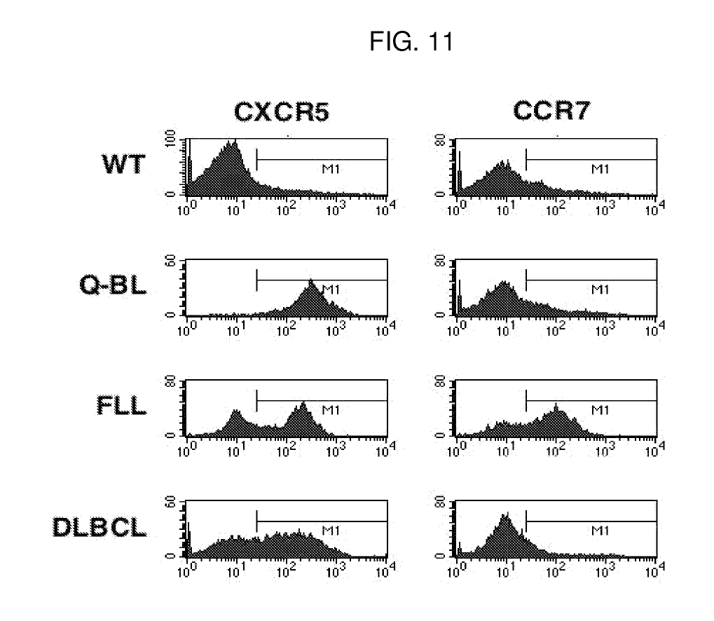

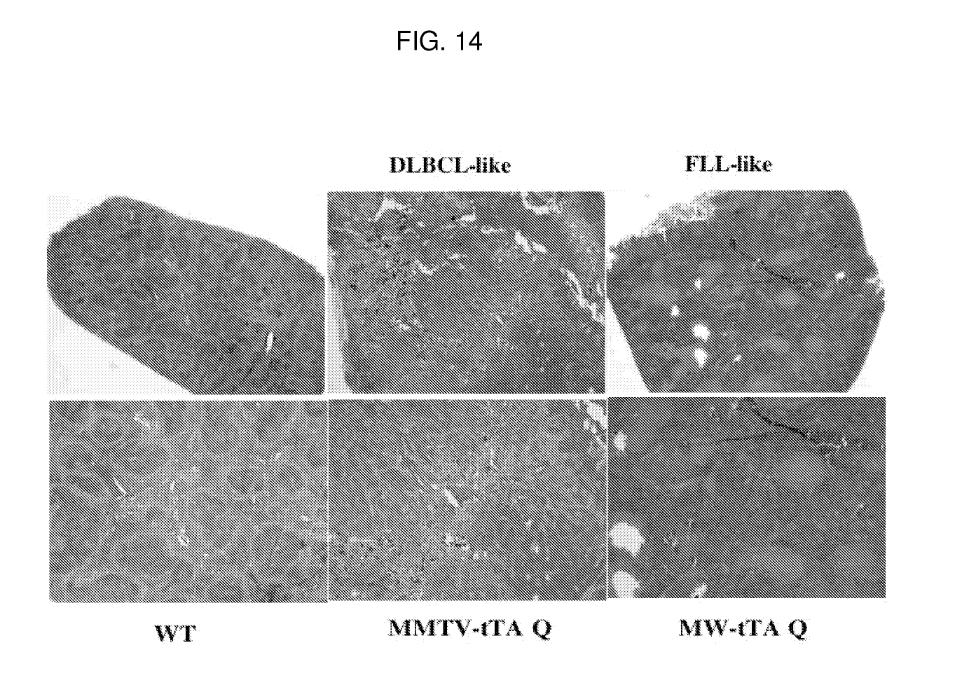

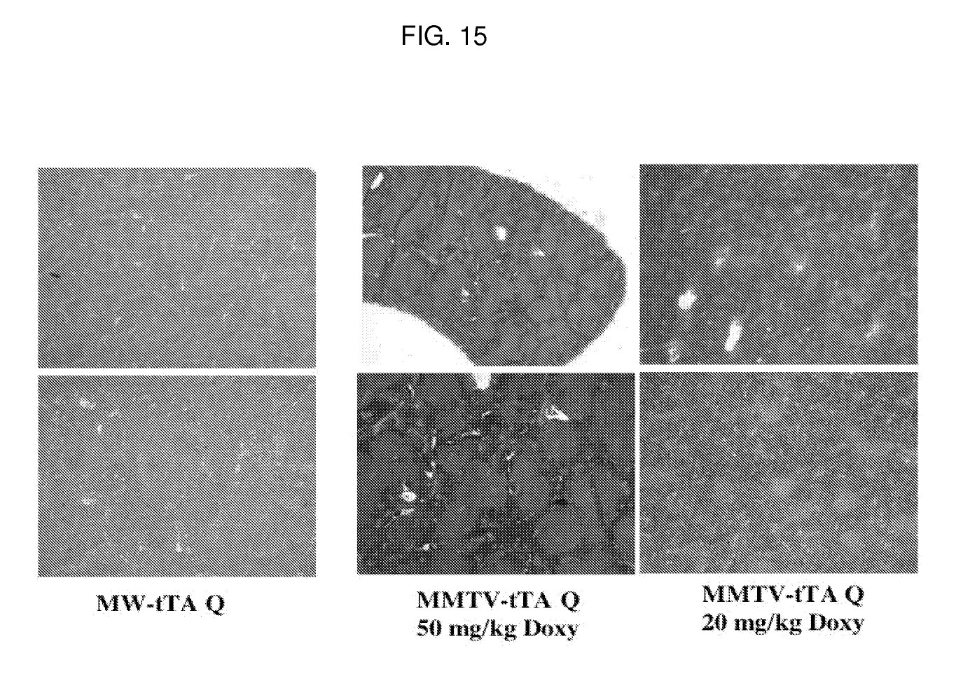

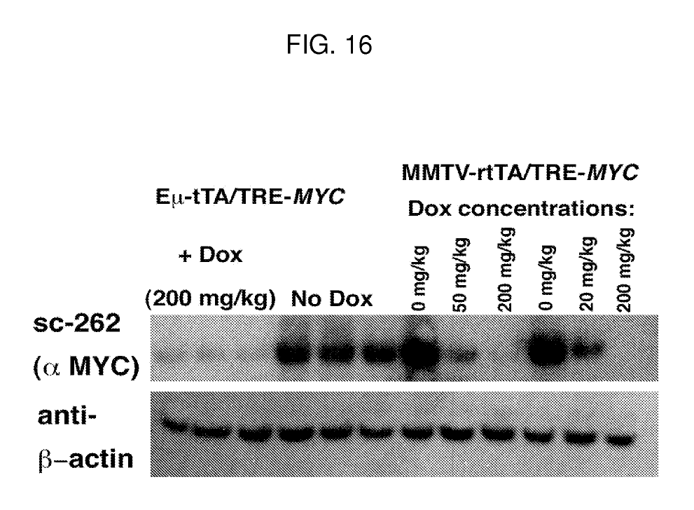

It is noted that this mouse strain is the same genetically as that described above in Example 2 with respect to Burkitt's Lymphoma (BL). However, the inventors have discovered that the overexpression of high levels of MYC in the context of autoreactive B-cells yields a different disease phenotype depending on the timing of transgene function. The overexpression of MYC in mice since birth yields a Burkitt's like tumor, including a unilateral jaw tumor (Example 2). In the FLL mouse model described in this Example, the overexpression of MYC in autoreactive B-cells in adult mice (>4 months of age) yields a tumor with extensive splenomegaly and lymphadenopathy and diffuse infiltration of the liver, but no jaw tumors (data not shown). Overgrowth of B-cells could be detected in the bone marrow, lymph nodes, spleen and thymus (FIG. 8 and Table 1). B-cells also infiltrated the liver, lungs, and central nervous system. Compression and invasion of the spinal cord caused paralysis of the hind and fore limbs. Histological examination revealed a homogeneous population of large lymphocytes in the spleen, lymph nodes, thymus and bone marrow (FIGS. 9 and 10). The histological appearance of the tumors was similar to that observed in human Follicular-like lymphoma (FLL) tumors, including the replacement of complex cell populations contained within the lymphoid follicles with sheets of monomorphic and anaplsatic large B-cells (Table 6). In addition, the inventors also noted a graded increase in the size of the follicular pattern of the B-cell lymphoma, as the tumors progressed, a feature prominently observed in human FLL (FIGS. 9 and 10). Referring to FIG. 10, the DLBCL like tumors exhibited sections which contained the diffuse and anaplastic activated B-cells as well as fibrotic tissue (DLBCL.1) or infiltrating activated T-cells (DLBCL.2). The surface phenotype of MMTV-rtTA/TRE-MYC/BCR^{HEL}/sHEL tumor cells also resembled that of Eμ-MYC/BCR^{HEL}/sHEL tumors, as was also consistent with the phenotypes described for the post-germinal center subtype of FLL (Tables 1 and 7 and FIG. 11). One additional aspect of these studies was the inventors' ability to convert an FLL like tumor to a DLBCL-like tumor by completely withdrawing the mice from doxycycline after the initial FLL like tumor arose (FIGS. 14, 15 and 16; see also Example 4).

In summary, the overexpression of high levels of MYC in the context of autoreactive B-cells in adult mice altered tumorigenesis by MYC in a distinctive manner. The distinctions involved diverse features of the tumors, including rate of appearance, anatomical presentation and progression, histopathology, and immunophenotype (Table 6). The tumors that arose in the presence of antigen stimulation resembled FLL in multiple ways.

TABLE 6
Comparison of human and mouse tumors

| | | | Anatomical | | | |
|---|---|---|---|---|---|---|
| | Latency | | presentation | | | MYC |
| | to onset¹ | Aggressiveness | & evolution | Histopathology | Immunophenotype | overexpression |
| Follicular- | >55 years | Indolent, but can | lymph nodes, | Anaplastic | Mature, | Yes, but not |
| like | of age | convert to an | spleen, bone | blasting cells | activated B | necessarily by |
| B-cell | | aggressive | marrow, | growing in a | cells (post- | chromosomal |
| Lymphoma | | DLBCL form. | central | follicular patter | germinal | translocation |
| (FLL) | | | nervous | in the lymphoid | center | |
| | | | system, | organs | type). | |
| | | | gastrointestinal | | | |
| | | | tract | | | |
| MMTV- | 8 weeks | Indolent, unless | lymph nodes, | Anaplastic | Mature, | Yes, |
| rtTA/ | after | completely | spleen, bone | blasting cells | activated B | MMTV- |
| TREMYC/ | activation | withdrawn from | marrow, liver, | with growing in | cells, post- | rtTA/TRE-MYC |
| BCR^{HEL}/ | of MYC | Doxycycline | CNS, lungs² | a follicular | germinal | transgene |
| sHEL | transgene | | | pattern in the | center | activated in |
| transgenic | in adult | | | lymph nodes | cells. | adult mice |
| mice | mice | | | and spleen³ | | |

¹Mean age of highest incidence in humans (Grogan et al, 1982; Rowe et al, 1985). The latency for the murine tumors is defined as the median of the data.
²Documentation of anatomical locations was determined by flow cytometric analysis for B220+/IgM− cells for Eμ-MYC tumors, B220+/IgM^{a}+ cells for Eμ-MYC/BCR^{HEL}/sHEL tumors and B220+/BCR^{HEL}+ cells for MMTV-rtTA/TRE-MYC/BCR^{HEL}/sHEL tumors. Anatomical distribution was confirmed by histological examination.
³The tumors in the Eμ-MYC mice consisted of a mixed population of small cleaved and non-cleaved lymphoid cells, as well as larger centrocytes.

TABLE 7
Cell surface phenotypes of different murine tumors, as defined
by flow cytometric analysis.
Immunophenotypic characteristics of the murine B-cell
lymphomas developed in quarduply transgenic mice

| Antigen | TBL | QBL | FLL | DLBCL |
|---|---|---|---|---|
| B220 | + | + | + | + |
| IgM | + | + | + | + |
| IgD | + | + | + | +/− |
| Hy-9 | + | + | + | + |
| (HEL) | | | | |
| CD21 | low | − | low | −/low |
| CD23 | + | + | + | + |
| CD69 | + | + | + | + |
| B7-2 | + | + | + | + |
| CD5 | −/low | − | − | −/low |

The Response of Tumors to Immunosuppression

To determine if this murine model of lymphomas would be useful for the testing of novel anti-cancer therapeutics, the inventors tested the effect of several immunosuppressants on existing tumors. Cyclosporin A, FK506, and rapamycin were injected to treat well advanced tumors that had been initiated by transplantation. The effects of these agents were compared to that of cyclophosphamide, an agent commonly used to treat human DLBCL (Schiffer, 2001).

10⁶ cells obtained from tumor-bearing spleen or lymph nodes were transplanted into cohorts of 4-10 mice. The recipient mice were held for observation until they developed externally obvious lymphadenopathy (approximately 100 days for the Eμ-MYC tumors, 21 days for the Eμ-MYC/BCR^{HEL}/sHEL tumors, and 14 days for the MMTV-rtTA/TRE-MYC/BCR^{HEL}/sHEL tumors). The tumor bearing and control wild-type mice were then treated daily for seven days with intravenous injections of the various drugs. Mice were either euthanized 24 hours after the last injection of drug, or held indefinitely to ascertain survival. The analysis of tumor burden was performed with cells obtained from lymph nodes and spleens.

The Eμ-MYC tumors did not respond to any of the immunosuppressive drugs tested. Disease progressed at the same rate in treated and untreated mice. Histological examination of the affected organs also revealed no evidence of therapeutic response (data not shown). In contrast, the transplanted Eμ-MYC tumors showed a strong response to cyclophosphamide, as previously described (Schmitt et al, 2002). Treatment with cyclophosphamide elicited tumor regression in all animals, but also caused a more general cytotoxicity, manifested as a reduction in T-cells, myeloid cells, and non-transgenic B-cells (data not shown). Similar toxicity from cyclophosphamide was also observed in wild type mice.

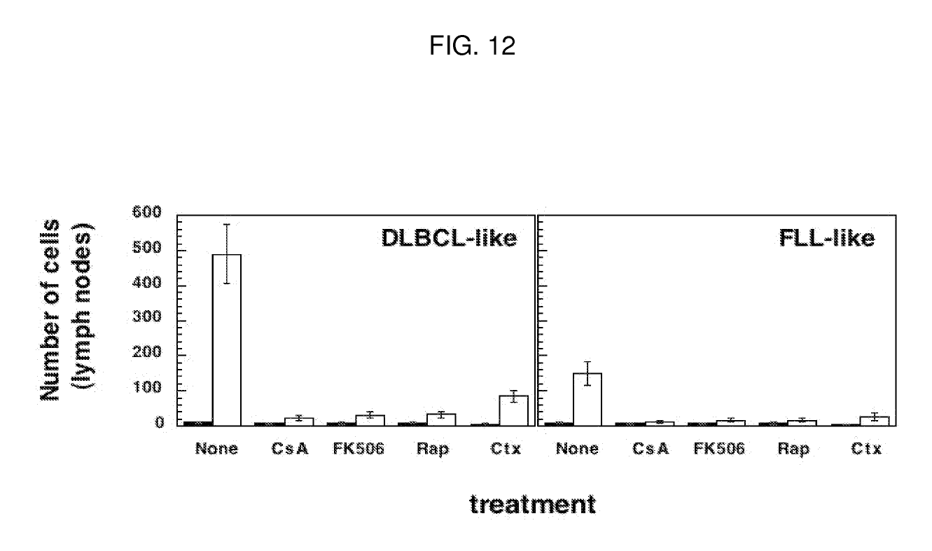

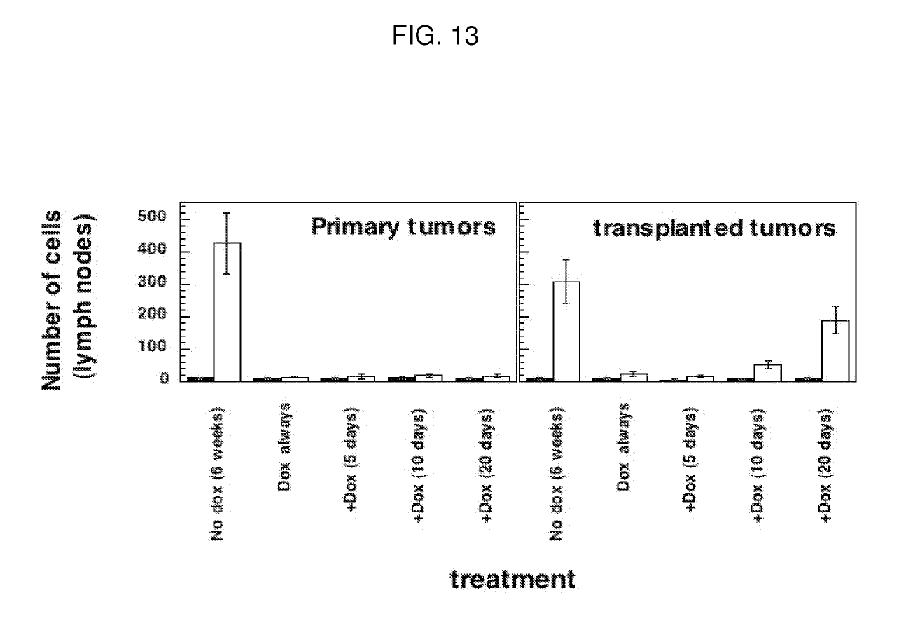

The tumors from Eμ-MYC/BCR^{HEL}/sHEL mice, as well as tumors from MMTV-rtTA/TRE-MYC/BCR^{HEL}/sHEL mice, responded to both cyclophosphamide and all three of the immunosuppressants tested (FIG. 12). In addition, these tumors also went into remission when tumor bearing mice were switched to a doxycycline containing diet, and the TRE-MYC transgene was suppressed again (FIG. 13).

Remissions of MMTV-rtTA/TRE-MYC/BCR^{HEL}/sHEL tumors persisted for at least 6 months, following a seven-day course of treatment with immunosuppressants (data not shown). In contrast, the animals treated with cyclophosphamide entered a brief remission, but still died more rapidly than did untreated, tumor-bearing mice (data not shown), apparently consequent to the toxicity described above.

A Mouse Model for Follicular-Like B-Cell Lymphoma

The experiments above describe a novel mouse model that develops a lymphoma with a close resemblance to human FLL. The similarities include anatomical presentation and other clinical manifestations, histological appearance, and immunophenotype. Several previous reports have described experimental approaches that were aimed at developing mouse models of FLL (Adams et al, 1985; Schmidt et al, 1988; Nussenzweig et al, 1988; Huang et al, 1995; Kovalchuk et al, 2000; Ruf et al, 2000). None of these attempts, however, produced a tumor with substantive resemblances to FLL. Most models aim to overexpress a single oncoprotein, or decrease expression of a single tumor suppressor gene, in the context of the B-cell compartment. No provision has been made for deliberate antigenic stimulation of B-cells, even though some of the rare tumors that result contain cells that show evidence of immune selection, in the form of point mutations in the Ig loci. In addition, most of those models consist of tumors that have a penetrance lower than 20% over an 18-24 month period.

Lymphomas constitute a number of different diseases that have been subdivided into two broad categories, Hodgkin's and Non-Hodgkin's lymphoma (NHL). Hodgkin's disease comprises a uniform set of malignancies primarily defined by the presence of Reed-Sternberg giant cells, whereas NHL is a heterogeneous set of clinical entities. The number of cases of NHL is almost ten fold that of Hodgkin's disease (Staudt, L. M. (2003). N Eng J of Med 348, 1777-1785). In addition, the number of newly diagnosed cases of NHL has increased by almost 80% in the last 25 years. This dramatic increase in newly diagnosed cases does not correlate with age, gender or infectious agents, and cannot be accounted for by the onset of HIV-associated B-cell lymphomas (Staudt, L. M., and Wilson, W. H. (2002) Cancer Cell 2, 363-6). As a result, NHL currently account as the fifth most common form of cancer in the United States, after breast, prostate, lung and colon cancer. NHL is one of the few cancers whose incidence and mortality rates have risen in the past 35 years. Despite the increase in the incidence of NHL, the etiology of these lymphomas remains elusive, and current therapeutic approaches rely on traditional, non-specific chemotherapeutic approaches.

FLL is the second most common type of B-NHL that occurs in adults in the US. It alone accounts for about 25% of all hematological malignancies in adults. FLL has a highly variable clinical course, including some individuals that can live for up to 20 years after diagnosis with indolent tumors and those that succumb to the disease within 1 year. In addition, some of the indolent tumors are known to convert to the aggressive form at some point during the course of the disease. Follicular lymphomas are derived from post-germinal center B-cells and themselves develop in a follicular pattern in the lymphoid organs. One salient characteristic of human FLL is the high prevalence of the translocation t(14;18) in which the Bcl-2 gene is juxtaposed to the IgH locus and is hence overexpressed in all FLL cells. The “conversion” into a DLBCL-like disease is associated with a variety of genetic alterations including the finding of higher levels of MYC transcripts in DLBCL cells when compared to FLL cells. The inventors have been able to establish mouse tumors that have a post-germinal center cell surface phenotype, develop and grow in a follicular pattern in the lymphoid organs as an indolent tumor then change into an aggressive DLBCL-like disease upon an experimentally induced increase in the levels of MYC protein. These characteristics have collectively allowed the inventors to classify the tumors that arise in the MMTV-tTA/TRE-MYC/BCR^{HEL}/sHEL mice activated in adults as a mouse model of FLL. In fact, by manipulating the same three genetic elements in an experimental system (B-cell antigen receptor, cognate antigen and the temporal, tissue specific and magnitude of MYC overexpression), the inventors have been able to give rise to a series of mouse models that recapitulate a spectrum of human disease that encapsulates the large B-cell NHLs as a category. It is believed that these molecular interactions will be critical for the biological basis of the development of these tumors and may even extend to the biology of autoimmune, polyclonal lymphoproliferative diseases.

The diagnosis of NHL encompasses different clinical entities. Approximately 85-90% of all NHL in the U.S. consist of B-cell lymphomas (Harris et al., (1994) Blood 84, 1361-92.). Among the B-cell NHLs, aggressive lymphomas account for 45-50% of new diagnoses. The two most common forms of aggressive NHL are Diffuse Large B-cell lymphoma (DLBCL) and Burkitt's lymphoma (BL). These two types of malignancies involve neoplastic B-cells that have a surface phenotype which is consistent with that of mature, activated B-cells. Specifically, they express B-cell antigen receptors on their surface that contain mutations consistent with the process of affinity maturation during a germinal center reaction (Kuppers et al., (1997) Eur J Immunol 27, 1398-1405.). These cells also express other molecules that are normally expressed by post-germinal B-cells (Morse, H. C. 3^{rd}, Anver, M. R., Fredrickson, T. N., Haines, D. C., Harris, A. W., Harris, N. L., Jaffe, E. S., Kogan, S. C., MacLennan, I. C., Pattengale, P. K., and Ward, J. M. (2002). Blood 100, 246-258.). There are other B-cell NHLs that have a similar cellular composition. These include Follicular lymphomas, Mucosal Associated Lymphoid Tissue (MALT) lymphomas, and mantle-cell lymphomas (Harris, supra). All of these B-cell NHLs share a B-cell surface phenotype, including BCR expression. The nature of additional mutations, possibly involving oncogenes and tumor suppressor genes, has been postulated to explain their biological differences (Pasqualucci et al., (2001) Nature 412, 341-6.). The one characteristic they have in common, namely BCR expression and evidence of antigen-dependent activation, may suggest that the B-cell antigen receptor (BCR) and an antigen may have an important role in the genesis of these tumors.

The notion of a role for chronic inflammation in lymphomagenesis has been popular for many years. There are prior hints that antigenic stimulus can play a role in lymphomagenesis. First, retroviral infection of mice elicits T-cell lymphomas only in those strains of mice that can mount an immune response to the virus (McGrath, M. S., and Weissman, I. L. (1979). Cell 17, 65-75; Lee, J. C., and Ihle, J. N. (1981) Nature 289, 407-9). Second, infection with Helicobacter pylori is an apparent cause of human lymphomas in mucosal associated lymphoid tissue (MALT) and gut associated lymphoid tissue (GALT) (Jones, R. G., Trowbridge, D. B., and Go, M. F. (2001). Front. Biosci. 6, E213-26.). Treatment with antibiotics to eradicate infection elicits remission of these tumors, as if they might have been sustained by antigenic stimulus from the microbe (Casella et al., (2001). Anticancer Res 21, 1499-502; Montalban et al., (2001) Gut 49, 584-7.). Third, mice with graft versus host disease consequent to bone marrow transplantation frequently develop T-cell lymphomas; immunosuppression of the mice prevents the tumors (Gleichmann et al. (1971) Verh. Dtsch. Ges. Inn. Med. 77, 1153-4.). Fourth, chronic antigenic stimulation by infection may contribute to the genesis of Burkitt lymphoma (BL) (Burkitt, D. P. (1971). Epidemiology of Burkitt Lymphoma. Proc. R. Soc. Med. 64, 909-10; Chapman et al., (1995). Blood 85, 2176-81). Fifth, the gene expression profiles of diffuse large B-cell lymphomas resemble those of B-cells that have mounted a response to antigen (Alizadeh et al., (2000). Nature 403, 503-11), and the tumor cells display high affinity antigen receptors on their surface, as if they had been subjected to the selective pressure of an antigen (Kuppers et al., (1997). Eur J Immunol 27, 1398-1405; Ottesmeier et al., (1998). Blood 91, 4292-4299; Lossos et al., (2000). Proc. Natl. Acad. Sci. USA 97, 10209-13; Lossos et al., (2000). Blood 95, 1797-1803.). These findings prompt the hypothesis that an antigenic stimulus may cooperate with other tumorigenic influences in the genesis of lymphoma.

### Example 4

The following example describes the production of a novel mouse model for the study of lymphomagenesis and for preclinical testing of therapeutics to treat human Diffuse Large B-cell Lymphoma (DLBCL).

Antigenic Stimulation Altered Tumorigenesis by MYC

The inventors bred the BCR^{HEL} and sHEL transgenes into a strain of mice that expresses MYC in the B-cell lineage (MMTV-rtTA/TRE-MYC). This strain allows for the temporal and tissue specific overexpression of MYC in an autoreactive B-cell background. The final composite strain was designated MMTV-rtTA/TRE-MYC/BCR^{HEL}/sHEL. When the mice were maintained on a doxycycline-containing diet (MYC transgenes remain silent) for four months, and released from doxycycline, the mice developed externally evident lymphadenopathy and splenomegaly accompanied by other clinical signs that are consistent with the onset of lymphoid neoplasia (scruffy fur, gasping, ascending hind limb paralysis, and dehydration). It is noted that this mouse model is genetically identical to the mouse model of BL and FLL described in Examples 2 and 3, but to produce the mouse model of DLBCL, the mice are initially maintained on doxycycline as for the FLL mouse model, but then are completely released from the doxycycline, as compared to the FLL model, wherein mice are maintained on a low dose of doxycycline after the initial time period.

In this DLBCL model, the tumors developed with an average latency of 6.5 weeks after the de-repression of the MYC transgene in those mice. The tumors proved to be universally lethal in these mice. Overgrowth of B-cells could be detected in the bone marrow, lymph nodes, spleen and thymus (FIG. 8 and Table 1). B-cells also infiltrated the liver, lungs, and central nervous system. Compression and invasion of the spinal cord caused paralysis of the hind and fore limbs. Histological examination revealed a homogeneous population of large lymphocytes in the spleen, lymph nodes, thymus and bone marrow (FIGS. 9, 10 and 14). The histological appearance of the tumors was similar to that observed in human Diffuse Large B-cell lymphoma (DLBCL) tumors, including the complete effacement of the endogenous architecture of the lymphoid organs, and the replacement of complex cell populations with sheets of monomorphic and anaplsatic large B-cells (Table 8 and FIG. 14). In addition, the inventors also noted the infiltration of activated T-cells into those tumors, a feature prominently observed in human DLBCL (FIGS. 9, 10 and 14). The surface phenotype of MMTV-rtTA/TRE-MYC/BCR^{HEL}/sHEL tumor cells also resembled that of Eμ-MYC/BCR^{HEL}/sHEL tumors as was also consistent with the phenotypes described for the post-germinal center subtype of DLBCL (Table 1 and 8, and FIG. 11).

In summary, the overexpression of high levels of MYC in the context of autoreactive B-cells in adult mice altered tumorigenesis by MYC in a distinctive manner. The distinctions involved diverse features of the tumors, including rate of appearance, anatomical presentation and progression, histopathology, and immunophenotype (Table 8). The tumors that arose in the presence of antigen stimulation resembled DLBCL in multiple ways.

TABLE 8
Comparison of human and mouse tumors

| | | | Anatomical | | | |
|---|---|---|---|---|---|---|
| | Latency | | presentation | | | MYC |
| | to onset¹ | Aggressiveness | & evolution | Histopathology | Immunophenotype | overexpression |
| Diffuse | >55 years | Very | lymph nodes, | Anaplastic | Mature, | Yes, but not |
| Large | of age | aggressive | spleen, bone | blasting cells | activated B | necessarily by |
| B-cell | | | marrow, | with infiltrating | cells. ABC, | chromosomal |
| Lymphoma | | | central | activated T-cells | GC or PM | translocation |
| | | | nervous | | subtypes. | |
| | | | system, | | | |
| | | | gastrointestinal | | | |
| | | | tract | | | |
| MMTV- | 6.5 | Very | lymph nodes, | Anaplastic | Mature, | Yes, |
| rtTA/ | weeks | Aggressive | spleen, bone | blasting cells | activated B | MMTV- |
| TREMYC/ | after | | marrow, liver, | with infiltrating | cells, post- | rtTA/TRE-MYC |
| BCR^{HEL}/ | activation | | CNS, lungs² | activated T- | germinal | transgene |
| sHEL | of MYC | | | cells³ | center cells | activated in |
| transgenic | transgene | | | | (GC-like) | adult mice |
| mice | in adult | | | | | |
| | mice | | | | | |

¹Mean age of highest incidence in humans (Grogan et al, 1982; Rowe et al, 1985). The latency for the murine tumors is defined as the median of the data.
²Documentation of anatomical locations was determined by flow cytometric analysis for B220+/IgM− cells for Eμ-MYC tumors, B220+/IgM^{a}+ cells for Eμ-MYC/BCR^{HEL}/sHEL tumors and B220+/BCR^{HEL}+ cells for MMTV-rtTA/TRE-MYC/BCR^{HEL}/sHEL tumors. Anatomical distribution was confirmed by histological examination.
³The tumors in the Eμ-MYC mice consisted of a mixed population of small cleaved and non-cleaved lymphoid cells, as well as larger centrocytes.

The Response of Tumors to Immunosuppression

To determine if this murine model of lymphomas would be useful for the testing of novel anti-cancer therapeutics, the inventors tested the effect of several immunosuppressants on existing tumors. Cyclosporin A, FK506, and rapamycin were injected to treat well advanced tumors that had been initiated by transplantation. The effects of these agents were compared to that of cyclophosphamide, an agent commonly used to treat human DLBCL (Schiffer, 2001).

10⁶ cells obtained from tumor-bearing spleen or lymph nodes were transplanted into cohorts of 4-10 mice. The recipient mice were held for observation until they developed externally obvious lymphadenopathy (approximately 100 days for the Eμ-MYC tumors, 21 days for the Eμ-MYC/BCR^{HEL}/sHEL tumors, and 14 days for the MMTV-rtTA/TRE-MYC/BCR^{HEL}/sHEL tumors). The tumor bearing and control wild-type mice were then treated daily for seven days with intravenous injections of the various drugs. Mice were either euthanized 24 hours after the last injection of drug, or held indefinitely to ascertain survival. The analysis of tumor burden was performed with cells obtained from lymph nodes and spleens.

The Eμ-MYC tumors did not respond to any of the immunosuppressive drugs tested. Disease progressed at the same rate in treated and untreated mice. Histological examination of the affected organs also revealed no evidence of therapeutic response (data not shown). In contrast, the transplanted Eμ-MYC tumors showed a strong response to cyclophosphamide, as previously described (Schmitt et al, 2002). Treatment with cyclophosphamide elicited tumor regression in all animals, but also caused a more general cytotoxicity, manifested as a reduction in T-cells, myeloid cells, and non-transgenic B-cells (data not shown). Similar toxicity from cyclophosphamide was also observed in wild type mice.

The tumors from Eμ-MYC/BCR^{HEL}/sHEL mice, as well as tumors from MMTV-rtTA/TRE-MYC/BCR^{HEL}/sHEL mice, responded to both cyclophosphamide and all three of the immunosuppressants tested (FIG. 12). In addition, these tumors also went into remission when tumor bearing mice were switched to a doxycycline containing diet, and the TRE-MYC transgene was suppressed again (FIG. 13).

Remissions of MMTV-rtTA/TRE-MYC/BCR^{HEL}/sHEL tumors persisted for at least 6 months, following a seven-day course of treatment with immunosuppressants (data not shown). In contrast, the animals treated with cyclophosphamide entered a brief remission, but still died more rapidly than did untreated, tumor-bearing mice (data not shown), apparently consequent to the toxicity described above.

A Mouse Model for Diffuse Large B-Cell Lymphoma

The experiments above describe a novel mouse model that develops a lymphoma with a close resemblance to human DLBCL. The similarities include anatomical presentation and other clinical manifestations, histological appearance, and immunophenotype. Several previous reports have described experimental approaches that were aimed at developing mouse models of DLBCL (Adams et al, 1985; Schmidt et al, 1988; Nussenzweig et al, 1988; Huang et al, 1995; Kovalchuk et al, 2000; Ruf et al, 2000). None of these attempts, however, produced a tumor with substantive resemblance to DLBCL. Most models aim to overexpress a single oncoprotein, or decrease expression of a single tumor suppressor gene, in the context of the germinal center B-cells No provision has been made for deliberate antigenic stimulation of B-cells, even though some of the rare tumors that result contain cells that show evidence of immune selection, in the form of point mutations in the Ig loci. In addition, most of those models consist of tumors that have a penetrance lower than 20% over an 18-24 month period.

DLBCL is the most common type of B-NHL that occurs in adults in the US. It alone accounts for about 40% of all hematological malignancies in adults. There is a heterogeneity in the pathology and biological characteristics of DLBCL as a disease entity, suggesting that a number of different diseases have been grouped into a common diagnostic entity. This would also suggest that the different subgroups would respond to different therapeutic modalities. Gene expression profiling of a number of different DLBCL tumors has revealed three main subcategories that resemble the gene expression profiles associated with a germinal center B-cell (GCB-DLBCL), an acutely activated B-cell (ABC-DLBCL) or primary mediastinal B-cell tumor (PM-DLBCL). The tumors we have obtained in our mouse model have many features that are consistent with the GCB-DLBCL, including cell surface phenotype, histology, presence of somatic mutations in their immunoglobulin gene sequences, presence of high levels of mRNAs for activation-induced cytidine deaminase (AID) and Bcl-6. These characteristics have collectively allowed us to classify the tumors that arise in the MMTV-tTA/TRE-MYC/BCR^{HEL}/sHEL mice activated in adults as a mouse model of GCB-DLBCL.

### Example 5

The following example describes the use of the set of novel mouse models that resemble human B-NHL tumors of the present invention for preclinical testing of drug candidates that target human molecules. The introduction of the human protein in the invention mouse models, along with the inducible abrogation of the endogenous murine orthologue allows for the direct examination of efficacy of a drug targeting a human protein in our mouse model, rather than a proof of concept using the murine proteins.

Model

The inventors' goal was to set up a model that can be used for testing the efficacy of immunotherapy targeted at any of the cell surface molecules (alone, or in combination) described in 5 different mouse models of large B-cell lymphomas. These include: (1) the Eμ-MYC/BCR^{HEL} model of B-CLL (see Example 1) also the MMTV-rtTA/TRE-MYC/BCR^{HEL} model with doxycycline as a positive control); (2) the Eμ-MYC/BCR^{HEL}/sHEL model of Burkitt's lymphoma (BL; see Example 2); (3) the MMTV-rtTA/TRE-MYC/BCR^{HEL}/sHEL (early/high) model of Burkitt's lymphoma (BL; see Example 2); (4) the MMTV-rtTA/TRE-MYC/BCR^{HEL}/sHEL (late/low) model of Follicular-like lymphoma (FLL; see Example 3); and (5) the MMTV-rtTA/TRE-MYC/BCR^{HEL}/sHEL (late/high) model of Diffuse large B-cell lymphoma (DLBCL; see Example 4). In each case, the use of doxycycline-repressible transgenes allows for a built-in positive control in therapeutic trials. The inventors have also tested the responsiveness of these mouse models to a therapeutic agent that is currently used in humans, cyclophosphamide. These two drugs provide a sufficient number of positive controls that can be used to compare the ability of the test agents to cause remission. In addition, the experiments are set up with retrovirally transduced cell lines derived from each of the tumor models discussed above, in order to ask direct questions of tumor maintenance, or as retroviral bone marrow chimaeras, in order to determine whether the antibodies can be used to differentially target the transformed B-cells, sparing the endogenous B-cell compartment. The latter studies will also shed light on the physiological function of the molecular targets in question.

Criteria and Assays to be Used for the Analysis of Therapeutic Potential of Drug Candidates

Gross examination of mice, photographic and filmed evidence of disease progression and/or regression
Necropsy and evaluation of tumor infiltration
Collection of lymph nodes, spleens, thymii and bone marrows for further analysis
Collection of liver, kidney, lung and sternii for histological examination
Determination of cell numbers present in the lymphoid organs collected
Flow cytometric analysis for normal and tumorous B-cells in the LN, SP, Thy and BM
Histopathology of the lymph nodes, spleens, thymii, sternii, livers and kidneys (CNS only if paralysis is observed in non-treated tumor bearing mice)
ELISA on sera samples for total Ig levels, HEL-specific Ig levels
Immunofluorescence of the kidneys for immune-complex deposition.
   Study Design

Analysis of Immunotherapy of Established Murine B-Cell Tumors Expressing a Human Cell Surface Protein.

In order to generate normal and transformed B-cells that express the human forms of cell surface proteins, such as CD22, CD20, DR5, CD79a or CD79b, the given cDNAs are introduced into one of three different variants of pMSCV vectors. The plasmids to be used include pMIG, which uses eGFP as a reporter gene, pMIT, which uses thy1.1 as a reporter gene, and pMICD8w, which uses a GPI-linked form of human CD8 as a reporter gene. The pMIG-based retroviruses express the gene of interest in a bicistronic message as well as a marker gene, GFP. The inventors have previously shown that the levels of GFP expression correlate proportionally with the levels of gene expression for the first cDNA encoded by the virus (Refaeli et al., (1998) Immunity 8:615-23; Refaeli et al., (2002) J. Exp. Med, 196, 999-1005). The viruses can be used to transduce bone marrow derived hematopoietic stem cells (HSCs), as well as mature, primary T and B lymphocytes. The bone marrow derived HSCs can then be used to reconstitute lethally irradiated mice.

Two parallel sets of experiments are performed in order to ascertain the efficacy of immunotherapy to any one of those cell surface molecules expressed on established tumor cells. The first approach involves the transduction of established tumor cell lines derived from MMTV-rtTA/TRE-MYC/BCR^{HEL}/sHEL tumors (either BL, FLL or DLBCL). The retroviral-mediated transduction is carried out in vitro. The level of infection is determined by flow cytometric analysis of reporter gene expression as well as surface expression of the respective cDNA. The ability to compare transduced and non-transduced cells in the same experiment allows for an important built-in negative control. The mixed population is then either maintained in culture, or used for adoptive transfer into cohorts of recipient mice.

The cells kept in culture are subjected to FACS analysis every 48 hours in order to determine if the transduced cDNA has an effect (either confers a competitive advantage, or a disadvantage) on the established tumor cell lines. A portion of these cells are also cultured in the presence of the conjugated antibodies in order to confirm their specific targeting of the transduced cells. In the event that the retroviral mediated expression of a human cell surface protein in a murine tumor cell is deleterious, due to gene-dosage effects, it may be necessary to either screen for retroviral clones that yield a lower level of expression. In addition, one may generate lentiviruses that encode shRNAs for the murine form of the gene. In that instance, the expression of the murine gene can be knocked down, and the expression of a human form of the gene driven by retrovirus mediated methods. This gene-replacement approach eliminates any confounding issues that may arise from having the human and murine form of the genes expressed simultaneously. In addition, BJAB is a cell line that can be used as a benchmark for testing the efficacy of the antibodies and test agents in vitro, as well as compare the levels of human protein expression on the surface of the murine lymphoma cells.

The transplant recipient mice are observed daily until they develop externally evident lymphadenopathy, as well as the clinical signs associated with the onset of hematological malignancies. These include scruffy fur, lethargy and sluggish movement, dehydration, gasping, hind limb paralysis of an ascending nature, externally evident lymphadenopathy and splenomegaly. It typically takes 3-4 weeks for recipient mice to develop clinical signs of disease.

Once the cohorts of mice show signs of illness, the various cohorts are treated as follows. One group will be left untreated, one will receive a previously agreed upon set of injections of the specific conjugated antibody, one will get a similar number of injections of a control antibody. In addition, doxycycline is used as a positive control. This agent will repress MYC expression in these tumors and lead to tumor regression, as the inventors have previously shown. Table 9 summarizes the various groups of mice needed such a study.

TABLE 9
An example of the total number of mice needed to test the efficacy of a
conjugated anti-human CD22 antibody to established B-NHL cell lines.

| | | | | # |
|---|---|---|---|---|
| | | Recipient | Therapeutic | of mice per |
| Donor cells | Retrovirus | mice | administered | group |
| MMTV- | None | Wild Type | None | 10 |
| rtTA/TREMYC/ | | | | |
| BCR^{HEL}/sHEL | | | | |
| (B-cell tumors) | | | | |
| | pMIG | Wild Type | None | 10 |
| | PMIG- | Wild Type | None | 10 |
| | CD22 | | | |
| | None | Wild Type | Conjugated | 10 |
| | | | anti-CD22 | |
| | pMIG | Wild Type | Conjugated | 10 |
| | | | anti-CD22 | |
| | PMIG- | Wild Type | Conjugated | 10 |
| | CD22 | | anti-CD22 | |
| | None | Wild Type | Conjugated | 10 |
| | | | control Ab | |
| | pMIG | Wild Type | Conjugated | 10 |
| | | | control Ab | |
| | PMIG- | Wild Type | Conjugated | 10 |
| | CD22 | | control Ab | |
| | None | Wild Type | +Doxycycline | 10 |
| | pMIG | Wild Type | +Doxycycline | 10 |
| | PMIG- | Wild Type | +Doxycycline | 10 |
| | CD22 | | | |
| Total number | | | | 120 |
| of mice needed | | | | |

The mice are harvested 24 hours after the last injection in order to determine the extent of the regression and ascertain the efficacy of the treatment. The mice are euthanized and the lymph nodes, spleens, thymii and bone marrow from two tibial and two femoral bones are harvested. These organs are used to generate single cell suspensions. The cells present in the cell suspensions are counted in order to estimate the number of cells contained in the various lymphoid organs. In addition, one of the kidneys of each mouse is collected and fixed in formalin, while the other kidney is frozen in OCT. A lobe from the liver is obtained, and the sterni, as well as additional lymph nodes, and a portion of the spleen and these are fixed in formalin. These organs are processed for staining with hematoxyllin and eosin in order to evaluate the histopathology of the lymphoid organs. In addition, the single cell suspensions obtained from the lymphoid organs are stained in order to analyze expression levels of B-cell developmental and activation surface markers. These include B220, thy1.2, CD19, CD21, CD23, CD43, PNA, B7-2 and CD69, in addition to staining for IgM, IgM^{a}, IgD and IgD^{a}. In addition, the human cell surface protein targeted by the specific immunotherapy used in these experiments is stained. The amount and specificity of the immunoglobulins present in the sera of those mice (i.e. whether they react to HEL) is also examined. These combinations of antibodies will allow the determination of the effect of MYC overexpression on HEL-reactive B-cells as well as on the endogenous, non BCR transgene-expressing cells (IgM+/IgM^{a}− cells). In addition, the examination of chromatin-reactive antibodies will allow the definition of whether the non BCR-transgenic B-cells have also circumvented normal tolerogenic mechanisms. The stains that label T-cells will allow the determination of whether these have become activated in the process of the MYC-driven breach of B-cell tolerance. Activated T-cells (defined by staining for CD3+/CD4 or CD8+/CD25, CD44 and CD69) will be identified to determine their appearance and possible increase in numbers during this process. In addition, a fraction of the single cell suspensions is used to generate cell extracts and perform western blots for MYC. It is expected that the remaining cells in any tumors found should be composed almost exclusively of the non-transduced cells.

In order to gain a better understanding of the kinetics and specific nature of each of the forms of immunotherapy to be tested in these systems, mice are bled every 2 days and the peripheral blood cells are stained for B220, IgM^{a}, IgM^{b}, B7-2, CD44, CD69, as well as for the retrovirally transduced human protein and thy1.2. In addition, sera samples are collected in order to determine the level of immunoglobulins in the sera of these mice, and whether they react with HEL and/or chromatin. These initial pieces of data allow for the monitoring of the dynamics of tumor regression in vivo, while enabling the following of a cohort of mice prospectively. It is possible that the recurrent bleeding of tumor-bearing mice may lead to the loss of some of the mice in each cohort. In order to mitigate this issue, the studies are preferably initiated with a larger number of mice in each of the groups (20-30 mice per group, instead of the 10 mice commonly used for single time point studies).

In a second approach, the experiments described in the previous section are mirrored, but rely instead on cells obtained from primary tumors. This will allow the testing of the efficacy of the specific immunotherapeutics in the context of a more heterogeneous population of cells, as is normally the case in human disease.

The experiments discussed thus far will determine the extent of the remission immediately after the end of the therapeutic regimen. In order to ascertain the duration of the remission that can be accomplished by the transient treatment of the tumor-bearing animal, the approach is slightly modified. In order to avoid relapses derived from the tumor cells that do not express the human cell surface molecule in question, a high-speed cell sorter is used in order to isolate the retrovirally transduced population of cells prior to transplantation.

The sorted, transduced tumor cells, or cell lines are then used for transplantation into recipient mice. The transplant recipient mice are allowed to develop lymphomas, as described, and treated as discussed previously and summarized in Table 9. The difference is that 24 hours after the end of the therapeutic regimen, one half of the remaining mice are harvested in order to document the efficacy of the initial treatment. The remaining animals are maintained in the animal facility and monitored daily for clinical signs associated with lymphoid malignancies. Any moribund animals are euthanized and the cause of illness will be evaluated to specifically attempt to determine if it is tumor-related. It will be important to determine if any relapses still express the cell surface protein in question for this therapy. This set of experiments will be greatly assisted by the use of whole-animal bioimaging technologies.

Analysis of the Effect of Immunotherapies on Mice that Express Human Surface Proteins on Normal and Transformed B-Cells.

The next set of experiments will determine whether the antibody based treatment of B-cell malignancies will also affect the endogenous B-cell compartment in mice. In these experiments, the same viral constructs defined in the experiments described above are used. In order to generate mice that express cell surface molecules on their normal B-cells, in the absence of generating a transgenic mouse strain for each one of the molecules in question, a set of bone marrow chimeric mice is used to ask this question. In order to distinguish between normal and transformed tumor cells, retroviral bone marrow chimeric mice are generated using donor cells from wild type, C57/BL6 mice, or MMTV-rtTA/TRE-MYC/BCR^{HEL} transgenic mice, on a C57/B6 background. The transduced BM cells are transplanted into lethally irradiated recipient mice, and allowed to develop, as previously described. All of those bone marrow transplant recipient mice will be maintained on a doxycycline-containing diet throughout their reconstitution period, in order to keep the TRE-MYC transgene repressed.

Analysis of the bone marrow chimaeras begins 8-12 weeks after transplantation. The bone marrow chimeric mice are bled and their blood stained with antibodies to B220, Thy1.2, Mac-1, Gr-1, NK1.1, IgM^{b} (endogenous IgM isoform in C57/B6 mice, and IgM^{a}-isotype specific staining for the BCR^{HEL} transgene). In addition, cells that express the specific lineage markers along with the retrovirally encoded reporter gene (GFP, thy1.1, hCD8) will be identified, if present, as well as the retrovirally encoded cDNA (hCD22, etc.). In cases in which a lentivirus encoding an shRNA specific for the murine orthologs of the human cell surface protein (e.g mCD22) is cotransduced, different reporters are used in the lentivirus and the retrovirus. The inventors currently have lentiviral vectors that express GFP, dsRED or Thy1.1 as reporters.

Once it has been established that the bone marrow cells engrafted appropriately, the bone marrow chimeric mice will be euthanized and their lymph nodes, spleen and bone marrow harvested. The bone marrows are used to reconstitute additional irradiated recipient mice in order to generate additional material for subsequent experiments. The lymph nodes and spleens are used to isolate purified B-cells, as previously described. Those cells are mixed at different ratios that can be followed in vivo by three criteria: a) expression of the retrovirally-encoded reporter genes, b) expression of the retrovirally encoded cDNA for the human cell surface protein and c) expression of IgM^{a} for the tumor-prone cells and IgM^{b} for normal B-cells. The mixtures are produced at four different ratios, based on previous experience of the inventors. 5×10⁶ cells are transferred to each of the mice in the cohort of mice to be generated in this fashion. The recipient mice are either wild type, C57/BL6 mice, or sHEL transgenic mice. In addition, the amount of doxycycline administered in their food, along with the timing of withdrawal of doxycycline from their food, will allow the use of a single pool of donor cells to test the effects of anti-hCD22 in mouse models of B-CLL, BL, FLL and DLBCL. Table 10 summarizes the number of mice needed for these studies.

TABLE 10
Number of mice needed to generate mixed BM chimeric mice.

| | | | Doxy- | # of mice |
|---|---|---|---|---|
| Donor Cells | Virus(es) | Recipients | cycline | per group |
| WT | pMIG | C57/BL6 | No | 10 |
| WT | pMIG-hCD22 | C57/BL6 | No | 10 |
| WT | pMIG-hCD22 + | C57/BL6 | No | 10 |
| | pLL3.77- | | | |
| | sh.mCD22 | | | |
| MMTV-rtTA/TRE- | pMIG | C57/BL6 | No | 10 |
| MYC/BCR^{HEL} | | | | |
| MMTV-rtTA/TRE- | pMIG-hCD22 | C57/BL6 | No | 10 |
| MYC/BCR^{HEL} | | | | |
| MMTV-rtTA/TRE- | pMIG-hCD22 + | C57/BL6 | No | 10 |
| MYC/BCR^{HEL} | pLL3.77- | | | |
| | sh.mCD22 | | | |
| Total number of | | | | 60 |
| mice needed | | | | |

Once the cohorts of mice that harbor normal and tumor-prone B-cells that express the human cell surface protein in question have been generated, they are allowed to develop tumors by withdrawing doxycycline from their diet. These mice will likely develop tumors 4-6 weeks later, as determined by the clinical signs associated with the onset of lymphomas. These mice are treated as described previously, with intravenous injections of the conjugated anti human CD22 antibodies, or other specific immunotherapeutics. The mice are analyzed as described in the experiments above. The additional aspect of this analysis is the inclusion of antibodies to IgM^{a}, IgM^{b} and hCD22, in order to specifically follow the fate of the normal and transformed murine B-cells that express hCD22. In addition, the fate of the IgM^{b}+, hCD22-B-cells will provide a built-in negative control, that consists of the endogenous murine B-cells that are not retrovirally transduced (the presence, or absence of the retrovirally encoded reporter gene will provide an additional level of confidence in this analysis). Table 11 summarizes the number of mice needed for one such experiment.

TABLE 11
Number of mice needed to test anti-hCD22 on endogenous and
tumorous B-cells. This example is for one B-cell tumor type
(DLBCL in this case). Similar number will be required for
testing the efficacy of anti-hCD22 on endogenous and
transformed B-cells in the context of BL and FLL.

| | | | | | # |
|---|---|---|---|---|---|
| | | | | | of mice |
| | | | | | per |
| Donor cells | ratio | recipients | Doxy | treatment | group |
| RV-WT | 100% | WT | No | None | 10 |
| RV-WT + RV- | 25:1 | WT | No | None | 10 |
| MMTV-rtTA/TRE- | | | | | |
| MYC/BCR^{HEL} | | | | | |
| RV-WT + RV- | 5:1 | WT | No | None | 10 |
| MMTV-rtTA/TRE- | | | | | |
| MYC/BCR^{HEL} | | | | | |
| RV-WT + RV- | 1:1 | WT | No | None | 10 |
| MMTV-rtTA/TRE- | | | | | |
| MYC/BCR^{HEL} | | | | | |
| RV-MMTV-rtTA/TRE- | 100% | WT | No | None | 10 |
| MYC/BCR^{HEL} | | | | | |
| RV-WT | 100% | WT | Yes | None | 10 |
| RV-WT + RV- | 25:1 | WT | Yes | None | 10 |
| MMTV-rtTA/TRE- | | | | | |
| MYC/BCR^{HEL} | | | | | |
| RV-WT + RV- | 5.1 | WT | Yes | None | 10 |
| MMTV-rtTA/TRE- | | | | | |
| MYC/BCR^{HEL} | | | | | |
| RV-WT + RV- | 1:1 | WT | Yes | None | 10 |
| MMTV-rtTA/TRE- | | | | | |
| MYC/BCR^{HEL} | | | | | |
| RV-MMTV-rtTA/TRE- | 100% | WT | Yes | None | 10 |
| MYC/BCR^{HEL} | | | | | |
| RV-WT | 100% | sHEL | No | None | 10 |
| RV-WT + RV- | 25:1 | sHEL | No | None | 10 |
| MMTV-rtTA/TRE- | | | | | |
| MYC/BCR^{HEL} | | | | | |
| RV-WT + RV- | 5:1 | sHEL | No | None | 10 |
| MMTV-rtTA/TRE- | | | | | |
| MYC/BCR^{HEL} | | | | | |
| RV-WT + RV- | 1:1 | sHEL | No | None | 10 |
| MMTV-rtTA/TRE- | | | | | |
| MYC/BCR^{HEL} | | | | | |
| RV-MMTV-rtTA/TRE- | 100% | sHEL | No | None | 10 |
| MYC/BCR^{HEL} | | | | | |
| RV-WT | 100% | sHEL | Yes | None | 10 |
| RV-WT + RV- | 25:1 | sHEL | Yes | None | 10 |
| MMTV-rtTA/TRE- | | | | | |
| MYC/BCR^{HEL} | | | | | |
| RV-WT + RV- | 5:1 | sHEL | Yes | None | 10 |
| MMTV-rtTA/TRE- | | | | | |
| MYC/BCR^{HEL} | | | | | |
| RV-WT + RV- | 1:1 | sHEL | Yes | None | 10 |
| MMTV-rtTA/TRE- | | | | | |
| MYC/BCR^{HEL} | | | | | |
| RV-MMTV-rtTA/TRE- | 100% | sHEL | Yes | None | 10 |
| MYC/BCR^{HEL} | | | | | |
| Total number of | | | | | 200 |
| mice needed | | | | | |

### Example 6

The following example describes the identification of the tec tyrosine kinase Syk as a novel molecular target for prophylaxis and treatment of B-cell non-Hodgkin's lymphomas, including B-cell chronic lymphocytic leukemia, Burkitt's lymphomas, Follicular like lymphomas and Diffuse large B-cell lymphomas, and further demonstrates as role for BCR signaling in lymphomagenesis.

Cooperation Between MYC and Constitutive or Cognate-Antigen Triggered BCR Signals in Lymphomagenesis.

The inventors propose herein an important role for BCR signaling in lymphomagenesis. To test this hypothesis, a transgene for BCR^{HEL} (40) was introduced into EμMYC mice (41), creating a strain of mice containing B-cells that both overexpresses MYC and has a known antigenic specificity (see Example 1). The Eμ-MYC/BCR^{HEL} bigenic mice are phenotypically normal at first, but develop lymphomas more rapidly than Eμ-MYC mice (Example 1, FIG. 1). Tumors in these mice differed from each other in their surface phenotypes, histopathology and anatomical distribution. The changes in the phenotype of those tumors are attributed to the contribution of the constitutive, or tonic signals that arise from the expression of the transgenic BCR. In order to examine how antigen stimulation of BCR^{HEL} contributed to lymphomagenesis, the inventors bred a ubiquitously expressed transgene for the cognate antigen, soluble Hen Egg Lysozyme (sHEL) (40) into the Eμ-MYC/BCR^{HEL} background (see Example 2). The triply transgenic mice (Eμ-MYC/BCR^{HEL}/sHEL) develop tumors even more rapidly than Eμ-MYC, or Eμ-MYC/BCR^{HEL} mice. The latter set of tumors also indicated that the overexpression of MYC in the context of a classic model of B-cell anergy (BCR^{HEL}/sHEL) resulted in the breach of immunological tolerance and the development of a lymphoma. The present inventors' now have additional data indicating that the breach of tolerance may result from the ability of elevated levels of MYC to rescue the expression of 10 Ca⁺²-sensitive genes whose expression is altered in anergic B-cells.

The inventors also bred the BCR^{HEL} and sHEL transgenes into a second strain of mice that express MYC in the B-cell lineage (MMTV-rtTA/TRE-MYC) (Hennighausen et al., (1995). J Cell Biochem 59, 463-72; Felsher et al., (1999). Mol Cell. 4, 199-207.)). The resulting strain was designated MMTV-rtTA/TRE-MYC/BCR^{HEL}/sHEL (Example 2, 3, 4). These mice offer the advantage of regulated MYC overexpression following withdrawal of doxycycline from the food of the animals. While the cellular compositions of the tumors developed in these mice is virtually identical to that observed in Eμ-MYC/BCR^{HEL}/sHEL mice, there is a striking difference in the initial presentation of their tumors. MMTV-rtTA/TRE-MYC/BCR^{HEL}/sHEL mice develop neoplasms in the jaw in a randomly unilateral manner (data not shown).

The disease eventually spread, affecting multiple organs. These observations eventually lead the inventors to conclude that the tumors developed in the Eμ-MYC/BCR^{HEL}/sHEL and MMTV-rtTA/TRE-MYC/BCR^{HEL}/sHEL mice resemble Burkitt's lymphoma of humans (BL), based on their histopathology, surface phenotype, anatomical site of presentation, age of onset, aggressiveness and mode of MYC dysregulation. The inventors propose that these mice are useful for preclinical studies of this disease.

Antigen-Dependence of the Murine B-Cell Lymphomas.

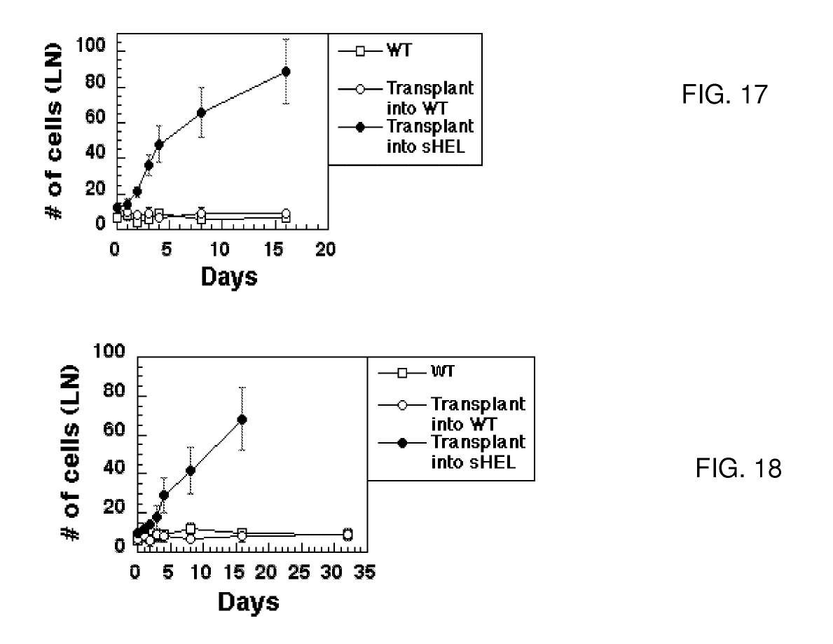

The inventors examined the specific contribution of antigen-dependent signaling to the initiation and maintenance of BL-like B-cell lymphomas by determining whether exogenous antigen could alter phenotypically normal EμMYC/BCR^{HEL} cells to resemble the tumor cells of Eμ-MYC/BCR^{HEL}/sHEL mice. Cells obtained from 4 week old Eμ-MYC/BCR^{HEL} mice, or 4 week old MMTV-rtTA/TRE-MYC/BCR^{HEL} mice were transplanted into either C57BL/6 mice, or age and sex matched sHEL transgenic mice. Donor cells were tracked by staining for B220 and HEL binding. In the absence of HEL, the number of donor cells did not change (FIG. 17), and the mice remained healthy. In contrast, the number of donor cells in sHEL recipients rose continuously over time, until the mice developed overt tumors. The phenotype of the tumor cells was similar to that observed in Eμ-MYC/BCR^{HEL}/sHEL mice (B220+, CD21−, CD23+, PNA+, B7-2+, CD69+, IgM+).

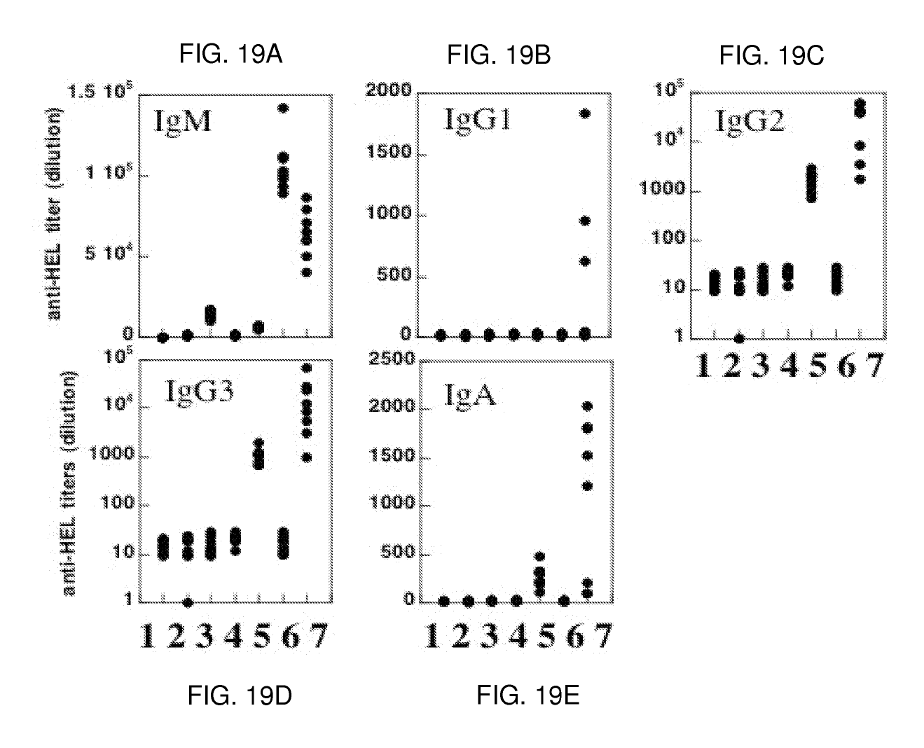

The inventors also tested the durability of the continuous requirement for antigen. For these experiments, tumor cells that had arisen after the initial transplantation of Eμ-MYC/BCR^{HEL} cells into sHEL mice were harvested and introduced into either wild type or a second set of sHEL mice. Donor cells amplified rapidly only in the sHEL mice (FIG. 18). The latency period was appreciably shorter than that observed after the preceding transplantation. In addition, the surface phenotype of the tumors that developed in these mice resembles that observed in Eμ-MYC/BCR^{HEL}/sHEL mice. Previous reports showed that BCR^{HEL} B-cells introduced into sHEL transgenic mice became anergic. The inventors' observations suggest that the overexpression of MYC in the BCR^{HEL} cells allows a response to the antigen, thereby creating a phenocopy of the tumors that developed in Eμ-MYC/BCR^{HEL}/sHEL mice and overcoming the tolerogenic control of autoreactive B-cell expansion.

Recent data provides independent evidence that the BL-like tumors the inventors observed in mice result from an antigen-driven process. Large B-cell lymphomas in humans are composed of B-cells that appear to have undergone a germinal center (GC) reaction. The presence of point mutations in rearranged Ig genes in those cells is consistent with their previous affinity maturation. In addition, cells from large B-cell lymphomas express transcripts of genes that appear to be involved in the GC reaction, such as Bcl-6. The BCR^{HEL} transgene used for the inventors' initial set of experiments has a high affinity for its cognate antigen (10⁻⁹M), and hence could not be used to determine whether the resulting tumor cells observed in Eμ-MYC/BCR^{HEL}/sHEL mice had experienced a GC reaction. In order to address this issue, a strain of mice in which a pre-rearranged VDJ segment, specific for HEL had been knocked into the IgH locus (VDJki), was obtained (Pape et al., (2003) J Exp Med 197, 1677-87). When these mice are used in combination with an Ig-light chain transgene (Lt-tg), the resulting mice (VDJki/Lt-tg) generate a large number of cells with a known specificity (HEL). The affinity of those receptors is low, but can be improved by an immune response (Pape et al., (2003) J Exp Med 197, 1677-87).

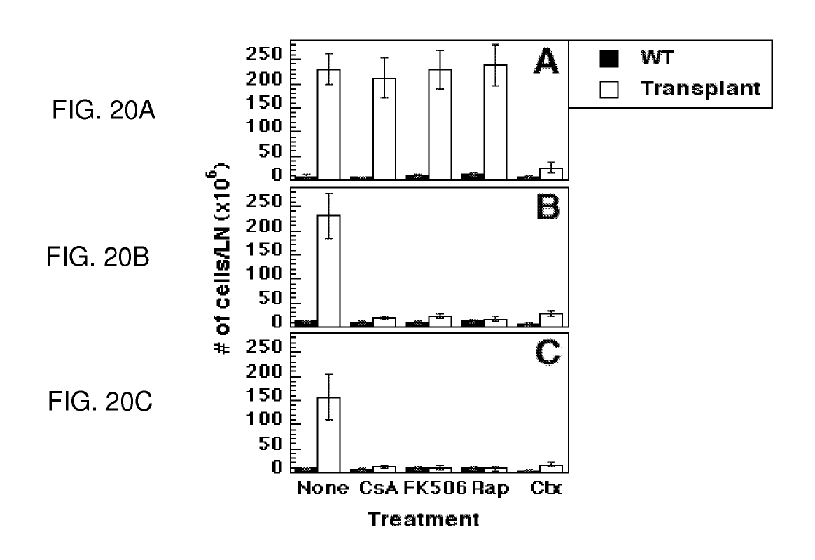

The inventors thus generated a strain of mice that was Eμ-MYC/VDJki/Lt-tg/sHEL, to examine whether the cells that composed the resulting BL-like tumors had undergone a GC reaction. The new strain developed tumors with kinetics similar to Eμ-MYC/BCR^{HEL}/sHEL mice. In addition, those tumors were also composed of mature, activated B-cells, characterized by the binding of PNA, a marker normally observed to bind the surface of GC cells. One important difference observed between the tumors in the new strain of mice vs. Eμ-MYC/BCR^{HEL}/sHEL mice was the secretion of additional Ig types (FIG. 19). The appearance of HEL-specific IgA, IgG1, IgG2 and IgG3, with varying affinities provide supporting evidence to the concept that these cells are derived from a GC reaction. In addition, the inventors PCR-amplified and sequenced the VDJ segment of the VDJki allele in the cells derived from the BL-like tumors observed in the Eμ-MYC/VDJki/Lt-tg/sHEL mice. As shown in Table 12, base pair substitutions were observed in the sequence analyzed in the majority of the clones that were sequenced. In addition, this observation was extended to cells obtained from 8 independent tumors. The inventors did not observe the appearance of base pair substitutions, or other mutations in the VDJ sequences obtained from tumors that arose in Eμ-MYC/VDJki/Lt-tg mice, in the absence of cognate antigen, suggesting that the observations the inventors have described thus far appear to arise as a result of an antigen-driven process (data not shown).

TABLE 12

Sequence analysis of VDJ joint region
cloned from Eμ-MYC/VDJki/Lt-tg/sHEL
tumor cells.

| | Sequence of the knocked-in | # of |
|---|---|---|
| Tumor/Clone | VDJ joint sequence | clones |
| 2918a | TACTCTG CAC ACTGG GACGGTGA | 12 clones |
| | TTACTGCGGCCAACGGTCTCTGGTC | |
| | TCTGTCTCTGCA | |
| | (SEQ ID NO:9) | |
| 2918b | TATTGTG CAA ACTGG GACCGTGA | 7 clones |
| | TTACCGCGGCCAAGGGACTCTGGT | |
| | CCCTGTCTCTGCA | |
| | (SEQ ID NO:10) | |
| 29180 | TACTGTG CAA ACTAA GACGGTGA | 3 clones |
| | TTACTGCGGCTAAGGGACTCTGGT | |
| | CACTGTCTCTGCA | |
| | (SEQ ID NO:11) | |
| 2564a | TACTGTG CAA ACTGG GACGGTGA | 8 clones |
| | TTACTGCAGTCAAGGGCCTCTGGT | |
| | CTCTGTCTCTGCA | |
| | (SEQ ID NO:12) | |
| 2564b | TCCTATG CAA ACTGG GACGGTGA | 2 clones |
| | TTACTGCGGCCAAGGGACTCTGGT | |
| | CACTGTCTCTGCA | |
| | (SEQ ID NO:13) | |
| NJC48a | TACTGTG CAA ACTGG GACGGTGA | 10 clones |
| | TTACTGCGGCCAAGGGACTCTGAT | |
| | CATTGTCTCGGCA | |
| | (SEQ ID NO:14) | |
| NJC48b | TACTGTG CAA ACTGG GACGATGA | 5 clones |
| | TTCCTGCGGCCTTGGGACTCTGGT | |
| | CGCTGTCTCTGCA | |
| | (SEQ ID NO:15) | |
| NJC81a | TACTGTG CAA ACTGG GACGGTGA | 9 clones |
| | TTACTGCTGCCACGGGTCCCTGGT | |
| | CCCTGTCTCTTCA | |
| | (SEQ ID NO:16) | |
| NJC81b | TACTGTG CAA ACTAG AACGGTGA | 6 clones |
| | TTACTGCGGCCAAGGGACCCTGGT | |
| | CACTGTCTCTGCA | |
| | (SEQ ID NO:17) | |
| NJC109 | TACTGTG CAA ACTGG GACGGTGA | 8 clones |
| | TTACTACGTCCAAGGGACTCTGGT | |
| | CACTGTCTCTGCA | |
| | (SEQ ID NO:18) | |
| Parental | V_{H}36-6O′ D N J_{H}3 | 20 clones |
| (Sequence of | TACTGTG CAA ACTGG GACGGTGA | |
| VDJki) | TTACTGCGGCCAAGGGACTCTGGT | |
| | CACTGTCTCTGCA | |
| | (SEQ ID NO:19) | |

Cells were obtained from tumor-bearing Eμ-MYC/VDJki/Lt-Tg/sHEL mice and used to obtain genomic DNA. The DNAs were used to set up a PCR reaction with primers surrounding the IgH VDJ joint region used for generating the knock-in mutation. PCR products were cloned into TOPO cloning vectors, and sequenced. The bold, underlined letters in the sequence show mutations found in the tumors, as opposed to the sequences obtained from a VDJki/Lt-Tg mouse (20 clones), presented at the bottom of the table.

While the class switching and somatic mutation data should suffice to formally prove that the BL-like tumors the inventors have developed in mice are composed of post-GC cells, the inventors also sought to determine whether those cells express transcripts of genes that are normally associated with the GC reaction. Accordingly, assays were developed for real-time, semi-quantitative RT-PCR for Bcl-6 and activation-induced cytidine deaminase (AID). Bcl-6 is highly expressed in human DLBCL, and a loss of function mutant in mice was shown to be defective in the formation of GC (46, 47). AID was shown to be critical for the processes of class-switch recombination and somatic mutation that are carried out during a GC reaction (16). The initial results show that 8/8 tumors analyzed thus far express high levels of Bcl-6 transcripts, and 5/8 tumors express high levels of AID mRNA relative to normal splenic B-cells (not shown). These observations can be verified by quantitating Bcl-6 and AID protein levels in the tumor cells. Taken together, these data show that the tumor cells in the BL-like tumors result from an antigen driven selection process and further support the previous observations concerning an important role for antigenic stimulation for tumor initiation and maintenance.

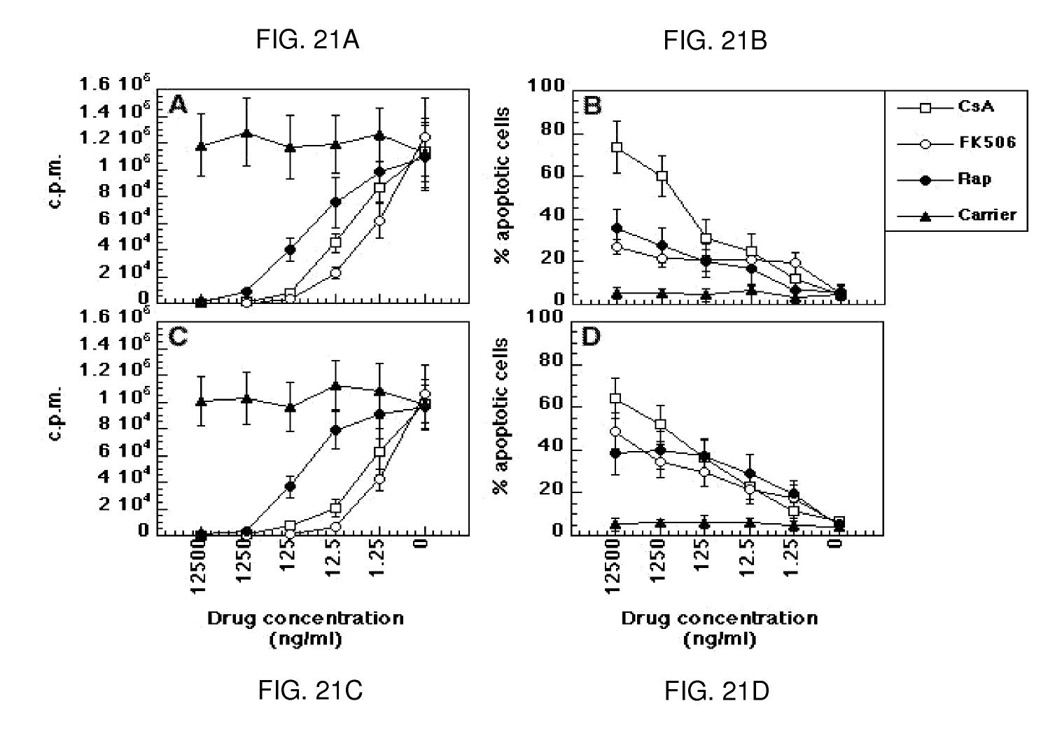

The inventors wanted to independently corroborate the dependence of murine B-cell lymphomas on BCR-dependent signaling pathways. To do so the effects of suppressing BCR signaling on mouse B-cell lymphoma models was determined. Cyclosporin A, FK506, or rapamycin were used to treat advanced tumors that had been initiated by transplantation. Effects of these agents were compared with the effects of cyclophosphamide, an agent commonly used to treat human BL (48). The Eμ-MYC tumors did not respond to any of the immunosuppressive drugs tested (FIGS. 20A-20C) with a similar rate of disease progression in treated or untreated mice. Histological examination of the affected organs also revealed no evidence of therapeutic response (data not shown). The transplanted tumors each showed a strong response to cyclophosphamide, as previously described (49). Strikingly, the tumors from Eμ-MYC/BCR^{HEL}/sHEL mice, as well as tumors from the jaws of MMTV-rtTA/TRE-MYC/BCR^{HEL}/sHEL mice, responded to all three immunosuppressants tested (FIGS. 20A-20C), suggesting that BCR signaling is a significant factor of disease progression. Treatment with cyclophosphamide elicited tumor regression in each instance, but also caused a more general cytotoxicity, manifested as a reduction in T cells, myeloid cells, and non-transgenic B cells (data not shown). Similar toxicity from cyclophosphamide was also observed in wild type mice. In addition, we were able to detect HEL mRNA in tumor cells by RT-PCR (data not shown).

Effects of Immunosuppressive Drugs in Vitro.

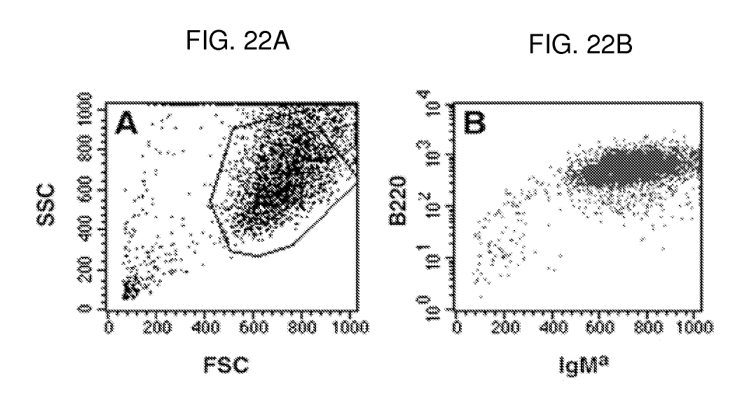

To determine the molecular basis of the interaction between BCR signals and MYC in lymphomagenesis, the inventors sought to establish in vitro systems that mimic relevant signaling elements in vivo. The effects of the three immunosuppressants used in vivo on primary and transformed B-cells were tested in vitro. Normal B-cells require the engagement of their BCR and a CD40 signals for their productive activation and proliferation in vitro. BCR^{HEL} B-cells cultured in vitro after stimulation with antibodies to IgM and CD40 displayed dose-dependent changes in proliferation and survival in the presence of Cyclosporin A, FK506 or Rapamycin (FIGS. 21A and 21B). Cells derived from Eμ-MYC/BCR^{HEL}/sHEL tumors proliferated in vitro without any added stimuli, and were similarly inhibited by the addition of immunosuppressants (FIGS. 21C and 21D). Together, these data indicate that proliferation of Eμ-MYC/BCR^{HEL}/sHEL tumors have retained a requirement for downstream mediators of BCR signaling.

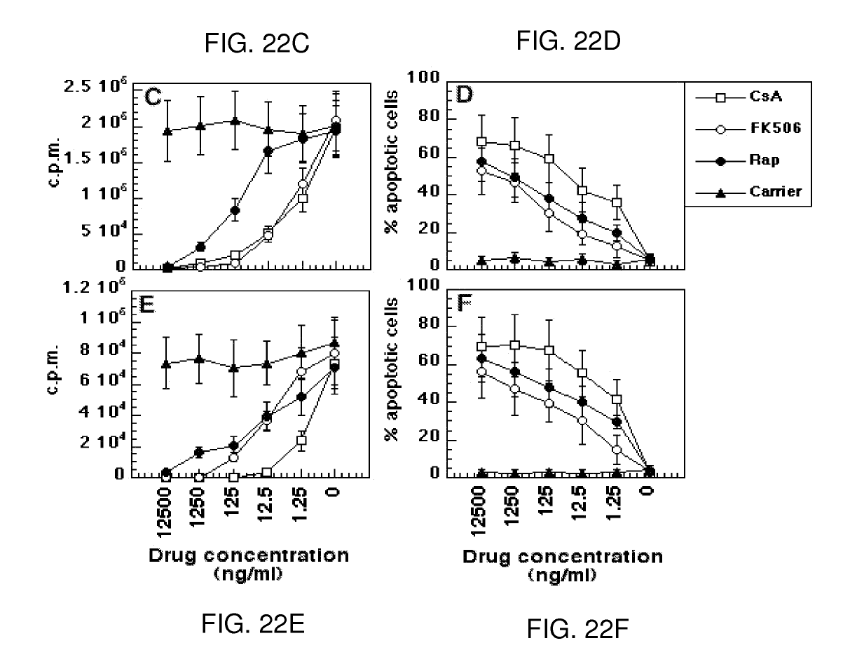

The inventors have established a number of B-cell lines from single cell suspensions obtained from primary malignancies. The tumorous B-cells were simply cultured in lymphocyte medium (RPMI 1640 supplemented with 10% Fetal calf serum, L-glutamine, penicillin/streptomycin, HEPES, sodium pyruvate, 2-mercaptoethanol, and non-essential amino acids) (50) for two weeks in 24-well plates. Resulting populations were expanded and were grown in continuous cultures for up to 6 months. In addition, the inventors have been able to freeze and recover them after thawing. These cell lines express the transgenic BCR (BCR^{HEL}; FIGS. 22A and 22B) and are susceptible to immunosuppression (FIGS. 22C and 22D). The inventors have also introduced some of these lines into C57BL/6 mice, developed tumors resembling those developed by the transplantation of primary malignancies (latency, histopathology, anatomical distribution, surface phenotype). The inventors will use these cell lines to define the mechanisms of BCR-dependent lymphomagenesis. These cell lines have provided the inventors with a system in which initial genetic analysis can be performed prior to carrying out studies in mice.

To validate the observations obtained with the murine tumors and cell lines, the inventors sought to determine whether cells from human Burkitt's lymphomas were also susceptible to the effects of the immunosuppressants. The effects of CsA, FK506 and Rapamycin were tested on three cell lines that were originally derived from Burkitt's lymphoma tumors, Raji, Ramos and Daudi (available from ATCC) (FIGS. 22E and 22F, and data not shown). Importantly, these cell lines share important features with the murine cell lines. First, they express a BCR on their surface. Second, they harbor translocations of MYC (t8;14) and express very high levels of MYC protein. Third, they were derived from the tumors prior to the treatment of these patients with any chemotherapeutics. Treatment with each of the three immunosuppressants resulted in dose-dependent decreases in proliferation and viability in vitro of the three cell lines, (FIGS. 22E and 22F and data not shown). The results generated with murine B-cells can now be validated with these three human cell lines. These preliminary tests may result in promising leads that are expected to prove to be useful for the treatment of primary human tumors.

Biochemical Analysis of Early Events in BCR Signaling in BL-Like Cells Revealed an Important Disconnect at the Level of Expression and Activation of Syk.

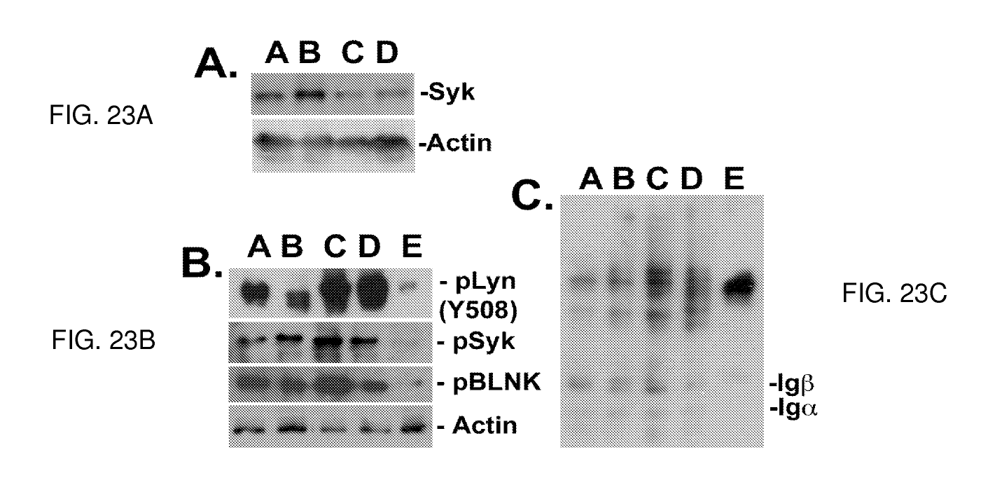

The inventors sought to determine whether there is a difference in the nature of the BCR-derived signals that arise from the BCR of formerly anergic B-cells that overexpress MYC. B-cells obtained from BCR^{HEL} mice were compared with either cells obtained from primary lymphomas (from both, Eμ-MYC/BCR^{HEL}/sHEL and MMTV-rtTA/TRE-MYC/BCR^{HEL}/sHEL mice). The normal B-cells were activated in vitro with antibodies to IgM and CD40, as the inventors have previously described. The inventors focused on the early events in BCR signaling, in order to determine whether the link to the Ca⁺² signaling pathway that was inferred from the genetic and pharmacological approaches described herein could be borne out biochemically. A few striking things were observed at this early stage in the analysis. First, the levels of Syk protein were reduced about three fold in the tumor cells, while the levels of Syk phosphorylation (Y523) were higher (FIGS. 23A and 23B). In addition, it was noted that while the levels of Lyn expression were comparable in normal and transformed B-cells, the levels of phosphorylation of a negative regulatory tyrosine residue (Y508) were much higher in transformed B-cells. These findings indicated that there was an aberration in the BCR-derived signal at the level of Syk. Interestingly, the inventors observed that there were many tyrosine-phosphorylated proteins in tumorous B-cells (FIG. 23C), including Igα and Igβ. Those results provide biochemical support for the biological data described herein that suggests that the BL-like B-cells are continuously receiving a signal derived from the BCR.

Validation of the Syk-Specific shRNA Encoding Lentiviruses.

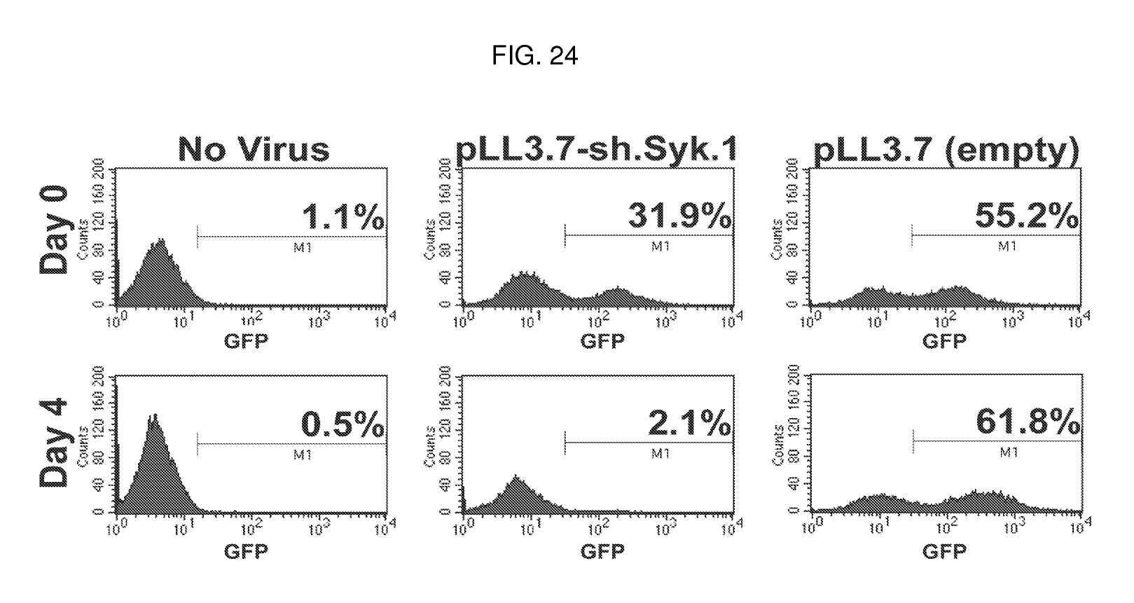

The inventors identified three sequences that met the various criteria defined previously by Angela Reynolds for predicting whether siRNAs were likely to target a specific sequence for degradation and knock down the resulting protein. The three sequences the inventors have identified scored 8 in the Reynolds scale (from 10 possible points). Oligonucleotides for the corresponding shRNA sequences and cloned these into pLL3.7 initially for validation purposes. The intent was to use a simple system for validation, and then clone the best performing sequence into pSICO, and other lentiviral vectors. The inventors initially tried to infect four different B-cell lines derived from murine BL tumors (TBL-1, TBL-8. TBL-9 and TBL-14). It was observed that while some GFP expressing cells could be seen immediately after the infection protocol was completed, the GFP+ fraction of the population was rapidly diluted and lost upon subsequent culture of those cells (FIG. 24). Referring to FIG. 24, TBL-8 cells were transduced with the indicated viruses, and analyzed by FACS on the last day of the transduction (day 0), or 4 days later. The cells harboring Syk-specific shRNAs were promptly out competed by their non-transduced counterparts. The inventors were trying to amplify the cells to a sufficient level that would allow us to perform a western blot for the protein (Syk) and a control (Actin). After repeated attempts, that also involved the use of high speed cell sorters in order to isolate the GFP expressing fraction of the population, it was realized that the reduction of Syk protein from those cell lines was being strongly selected against in vitro. While this was somewhat encouraging, because the shRNAs were exhibiting a biological effect, a better approach was needed to conclusively establish its effects of the levels of Syk protein.

Accordingly, the inventors then cloned the three shRNA encoding sequences into pSICO. GFP expression was initially validated in the four murine BL cell lines. Significant levels of GFP expression were seen that were stable over time in culture (data not shown). These murine BL cell lines were subsequently transduced in vitro with pSICO, or three Syk-specific shRNA encoding variants of pSICO (pSICO-sh.Syk.1, pSICO-sh.Syk.2 and pSICO, sh.Syk.3) as well as a retroviral construct that encodes CRE-ER, and expresses human CD8 as a marker (this is a mutant that has no cytoplasmic tail, and is maintained on the cell surface through a GPI-linked tag). The inventors used a high-speed cell sorter to specifically isolate the GFP+/CD8+ fraction of those cultures. The sorted cells were then incubated in vitro with IL-4 and 40HT for 48 hours, and used to generate cell lysates for western blotting.

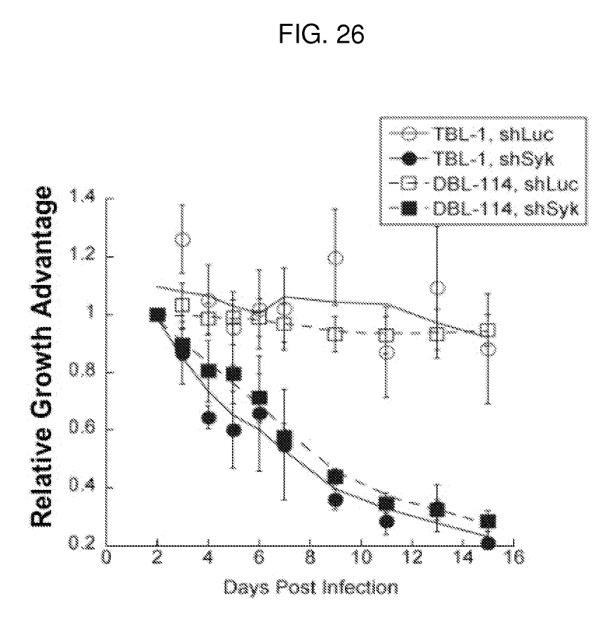

More specifically, referring to FIGS. 26A and 26B, protein concentrations of the lysates were determined using a Bradford assay, and equal protein amounts were loaded on each lane. The blots were probed with a rabbit polyclonal antibody to Syk, kindly provided by Dr. John Cambier, at National Jewish Medical and Research Center. Syk is approximately 72 kD. A second, non-specific band appears in the blot, which is a useful internal loading control. It is estimated the level of knockdown accomplished by the sh.Syk.1 shRNA was approximately 85%, using the NIH-Image program. The inventors generated variants of pSICO that encode the 3 Syk-specific shRNAs. All of these had a Reynolds score of 8. Those plasmids were used to generate infectious lentiviral particles, and used those to transduce TBL-8 cells (murine BL cell line). The cells were cotransduced with pMICD8-Cre.ER (pMSCV based virus encoding Cre.ER-IRES and a human CD8 reporter). The GFP+/hCD8+ cells were isolated using a high-speed cell sorter. These cells were cultured in media alone (pSICO, No 40HT added), or in the presence of 40HT (pSICO, +40HT).

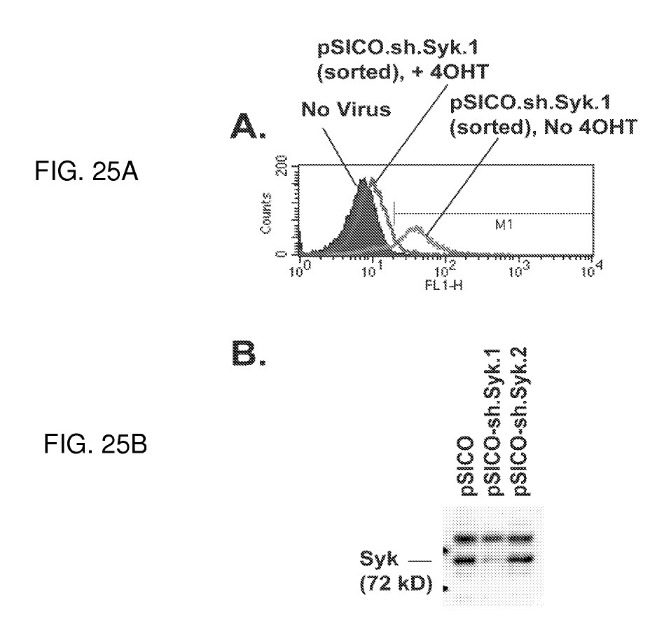

The levels of GFP expression in the sorted cells dropped significantly after their incubation in 40HT for 48 hours (FIG. 25A). Two of the three cultures transduced with Syk-specific shRNA encoding lentiviruses contained a sufficient number of cells for the subsequent analysis. The third construct did not. FIG. 25B shows the western blot data that confirmed that one of the Syk-specific shRNA sequences could very efficiently knock down Syk protein levels.

The inventors have now generated one, and perhaps two highly effective Syk-specific shRNA encoding lentiviruses. These sequences are currently cloned in pLL3.7, pSICO and pSICO-R, allowing for maximum flexibility in the inventors' studies. Using NIH-Image to quantify or western blot data, the inventors currently estimate that the sh.Syk.1 hairpin is able to knockdown 85% of the Syk protein found in B-cells. The ability of the lentivirally encoded shRNA to affect primary tumor development and maintenance is to be tested by generation of the appropriate bone marrow chimeric mice. In addition, the inventors have noted a critical role for Syk expression and function in B-CLL like tumors in mice. In addition, these experiments allow the initiation of studies to define the effect of disrupting Syk expression on the phosphorylation of BLNK, a downstream target. Those studies will be important to define the specific alteration of the activity on a molecular level. The inventors will also be able to compare those studies to other ongoing studies involving the knockdown of the Src-tyrosine kinase Lyn as well as the signaling components of the B-cell antigen receptor, Igα and Igβ.

Table 13 lists the relevant Syk nucleotide sequences used herein for mouse and human cells.

TABLE 13

| | | | |
|---|---|---|---|
| Reynolds | | | |
| score | core oligo | sense | antisense |

| | | | | |
|---|---|---|---|---|
| hSyk | | | | |
| 868 | 8 | GTCGAGCATT | TGTCGAGCATTATTCTTATAT | TCGAGAAAAAAGTCGAGCATT |
| | | ATTCTTATA | TCAAGAGATATAAGAATAATG | ATTCTTATATCTCTTGAATATAA |
| | | (SEQ ID NO:20) | CTCGACTTTTTTC | GAATAATGCTCGACA |
| | | | | (SEQ ID NO:32) |
| 1009 | 8 | GGAATAATCT | TGGAATAATCTCAAGAATCAT | TCGAGAAAAAAGGAATAATCT |
| | | CAAGAATCA | TCAAGAGATGATTCTTGAGAT | CAAGAATCATCTCTTGAATGAT |
| | | (SEQ ID NO:21) | TATTCCTTTTTTC | TCTTGAGATTATTCCA |
| | | | (SEQ ID NO:27) | (SEQ ID NO:33) |
| 2558 | 8 | AAGCTTTCCT | TAAGCTTTCCTGACAATAAAT | TCGAGAAAAAAAAGCTTTCCT |
| | | GACAATAAA | TCAAGAGATTTATTGTCAGGA | GACAATAAATCTCTTGAATTTA |
| | | (SEQ ID NO:22) | AAGCTTTTTTTTC | TTGTCAGGAAAGCTTA |
| | | | (SEQ ID NO:28) | (SEQ ID NO:34) |
| mSyk | | | | |
| | 8 | GGCAGCTAGT | TGGCAGCTAGTGGAACATTA | TCGAGAAAAAAGGCAGCTAGT |
| | | GGAACATTA | TTCAAGAGATAATGTTCCACT | GGAACATTATCTCTTGAATAAT |
| | | (SEQ ID NO:23) | AGCTGCCTTTTTTC | GTTCCACTAGCTGCCA |
| | | | (SEQ ID NO:29) | (SEQ ID NO:35) |
| | 8 | GCAACTTTGT | TGCAACTTTGTGCACAGAGA | TCGAGAAAAAAGCAACTTTGT |
| | | GCACAGAGA | TTCAAGAGATCTCTGTGCAC | GCACAGAGATCTCTTGAATCT |
| | | (SEQ ID NO:24) | AAAGTTGCTTTTTTC | CTGTGCACAAAGTTGCA |
| | | | (SEQ ID NO:30) | (SEQ ID NO:36) |
| | 8 | GTGGAACATT | TGTGGAACATTACTCTTACAT | TCGAGAAAAAAGTGGAACATT |
| | | ACTCTTACA | TCAAGAGATGTAAGAGTAAT | ACTCTTACATCTCTTGAATGTA |
| | | (SEQ ID NO:25) | GTTCCACTTTTTTC | AGAGTAATGTTCCACA |
| | | | (SEQ ID NO:31) | (SEQ ID NO:37) |

The inventors also examined the effects of genetically targeting Syk expression in established tumor cells, over time. An in vitro competition assay was performed to evaluate how targeted knockdown of Syk expression affected the fitness of tumor cell lines. Tumor cell lines derived from an Eμ-MYC/BCR^{HEL} tumor (DBL-114, DBL-120) or an Eμ-MYC/BCR^{HEL}/sHEL tumor (TBL-1, TBL-90) were infected with empty vector shLuciferase or shSyk.1. Initial infection efficiency was determined two days post-infection, and the percent of GFP positive cells relative to the empty vector control was monitored for two weeks. Tumor cell lines expressing shSyk.1 had a growth disadvantage relative to uninfected cells in the same well (FIG. 26, DBL-120 and TBL-90 data not shown). Comparable results were observed with shSyk.2 and shSyk.3 (Data not shown). Expression of the control shRNA, shLuciferase, had no significant effect on growth advantage, demonstrating that shRNA expression alone does not affect tumor B-cells (FIG. 26). Similar rates for loss of shSyk.1 expressing cells were observed for Eμ-MYC/BCR^{HEL} cells and Eμ-MYC/BCR^{HEL}/sHEL cells. Referring to FIG. 26, cells were transduced with either pLL3.7-shSyk or pLL3.7-shLuc. The transduction levels were evaluated by flow cytometry at day 0 and every 24 hours subsequently. The data are presented as the ratio of GFP+ cells in a given day divided by the starting level of GFP+ cells at day 0, in the live population. These results show that for both, DBL-114 and TBL-1 cells, the reduction in the levels of Syk expression result in a significant growth disadvantage in vitro, when compared with the negative control (shRNA for firefly luciferase).

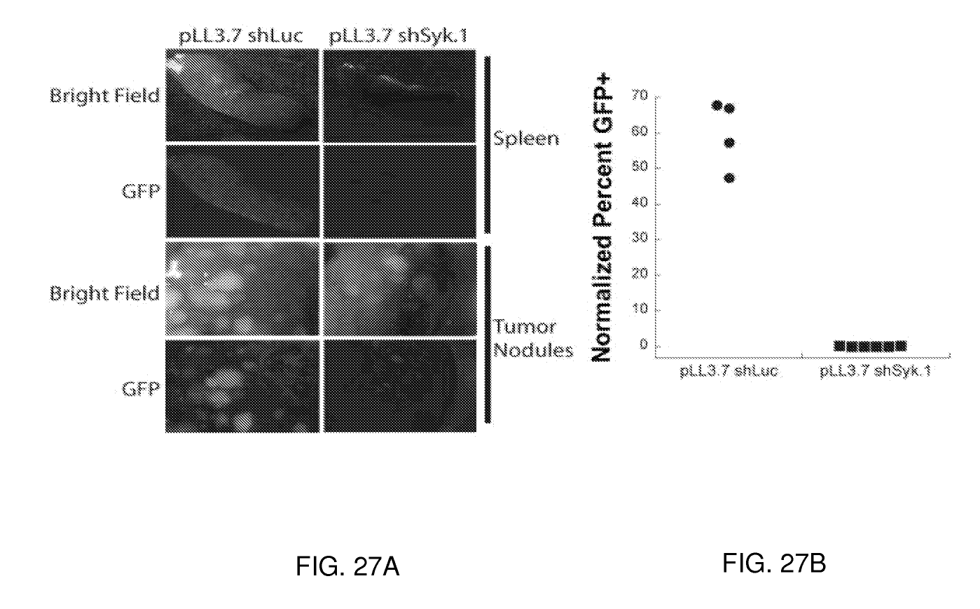

To investigate the validity of conclusions drawn from the in vitro competition assay, the inventors next examined how genetic ablation of Syk affects tumor maintenance in an in vivo competition assay. An Eμ-MYC/BCR^{HEL} tumor cell line (DBL-114) was infected with control shLuciferase or Syk-specific shRNA, shSyk.1. Four days post-infection, a mixture of infected (GFP+) and uninfected (GFP+) cells were adoptively transferred into Rag 1^{-/-} recipient mice. When mice developed externally evident tumors, they were sacrificed and lymphoid tumors harvested. The percent of GFP positive tumor cells from each mouse was determined by FACS analysis. Tumors harvested from mice that received DBL-114 cells expressing the control shLuciferase hairpin retained the majority of their GFP fluorescence (FIG. 27). The inventors routinely have observed some loss of GFP fluorescence with control shRNAs or vector alone, in agreement with other published results (Kissler et al., (2006). Nature Genetics 38, 479-83). In contrast, the inventors observed a paucity of GFP fluorescence in tumors harvested from mice transferred B-cell tumors expressing shSyk.1 (FIG. 27), indicating that Syk expression is necessary for maintenance of mature Eμ-MYC/BCR^{HEL} tumors in vivo. Referring specifically to FIGS. 27A and 27B, the inventors transduced B-CLL cells (DBL-114) with either pLL3.7 or pLL3.7-sh.Syk. The cells were transduced at a frequency of approximately 60%, as determined by flow cytometry, prior to injection into mice. 10⁵ transduced cells were transplanted into cohorts of Rag-1^{-/-} recipient mice, by tail vein injection. The mice were euthanized 21 days later and analyzed for tumor burden and GFP expression, by necropsy and fluorescence microscopy. Tumor masses were used to generate single cell suspensions and flow cytometry for GFP. FIG. 27A shows photographs in either brightfield, or with GFP fluorescence for a representative spleen and tumor nodules that form on the liver of tumor-bearing Rag-1−/− mice. The tumors that developed following the transplantation of DBL-114 cells transduced with pLL3.7 exhibited strong GFP expression and fluorescence. The Rag-1−/− mice that were given transplants of DBL-114 cells transduced with pLL3.7-sh.Syk still developed tumors, although presented with a lower tumor mass, and these tumors showed low levels of GFP expression and fluorescence. The summary of the flow cytometric data is presented in FIG. 27B.

Pharmacological Inhibition of Syk in Murine and Human BL Cell Lines Lead to a Rapid Decrease in Cell Viability, in a Dose-Dependent Manner.

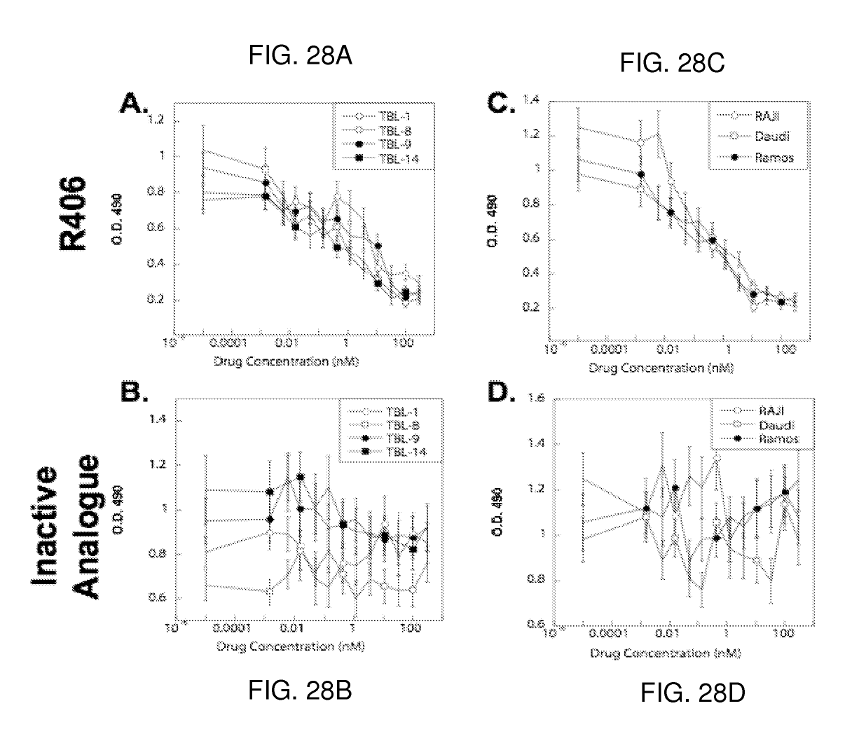

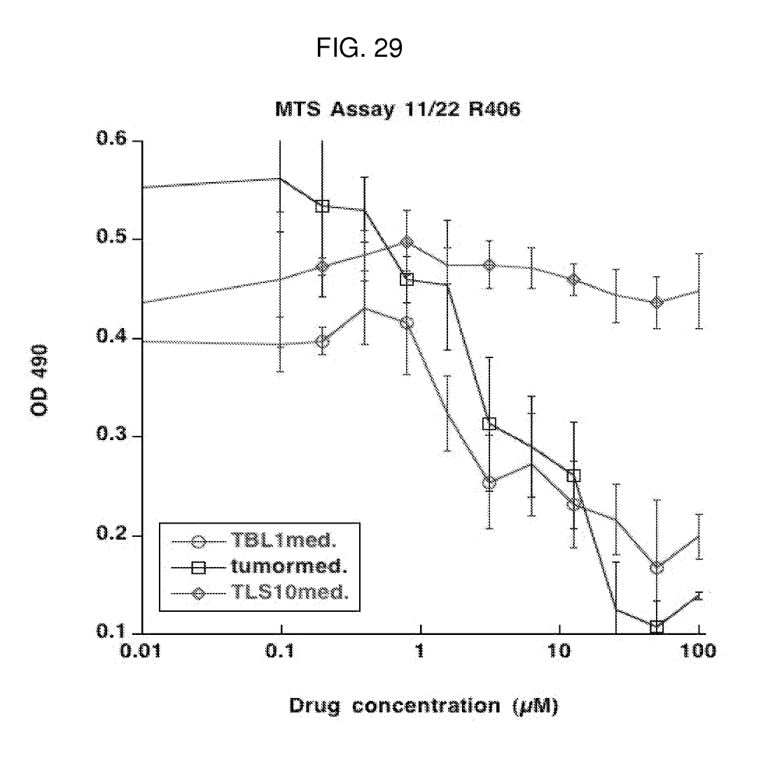

The inventors obtained a highly specific pharmacological inhibitor of Syk through a collaboration with Rigel Pharmaceuticals. The compound is called R406, and was shown to preferentially target the kinase function of Syk. The next kinase that could be inhibited with R406 required a much higher concentration of the compound. The inventors tested the effects of R406 on four murine BL cell lines (TBL-1, TBL-8, TBL-9 and TBL-14). Also tested were the effects of the Syk kinase inhibitor on primary murine BL the inventors obtained from triply transgenic mice (Example 2). In addition, the effects of R408 were tested on three cell lines derived from human BL tumors (Raji, Ramos and Daudi). The effects of R406 on the viability of all of these cells was tested using MTS assays (Promega, WI). 2×10⁴ cells were plated per well, in a 96 well plate, with increasing concentrations of R406, starting at 0.01 nM. The cells and drug were incubated at a final volume of 200 μl in each well. Each condition was set up in triplicate wells, in any one experiment. The cells were incubated in the presence of R406, or an inactive analogue compound, for 22 hours, and pulsed with 10 μl of MTS reagent for 2 hours, at 37° C. The plates were read with a Molecular Dynamics ELISA plate reader, at OD 490. The data presented in FIGS. 29A-29D is from one experiment, representative of three independent assays. The error bars represent the standard deviations. Referring to FIGS. 29A-29D, 2×10⁴ cells from the following murine BL cell lines: TBL-1, TBL-8, TBL-9, TBL-14 (FIGS. 29A and 29B), or the human cell lines (Raji, Ramos, Daudi; FIGS. 29C and 29D) in increasing concentrations of either a pharmacological inhibitor of Syk (R408; FIGS. 29A and 29C), or an inactive chemical analogue (FIGS. 29B and 29D), as a negative control. The cells were cultured for 22 hours with the compound, and pulsed for the last 2 hours with MTS reagent. The O.D.490 absorbance of the wells was determined 24 hours after the beginning of the experiment. All conditions were tested in triplicate wells. The error bars represent the standard deviations of the triplicate wells. These data were obtained from one experiment, representative of three independent assays.

Using this approach, four primary tumors derived from Eμ-MYC/BCR^{HEL}/sHEL mice have been tested, three cell lines derived from such tumors (TBL-1, TBL-8 and TBL-14), as well as a T-cell lymphoma cell line (iTLS-10). The T-cell lymphoma cell line served as a negative control for the Syk compound, since the inventors have shown that it does not express Syk, nor does it respond to other known inhibitors of the kinase (Piceatannol) (FIG. 29). Referring to FIG. 29, In order to determine the specificity of R406, and rule out any possible crossreactivity with the T-cell specific tec tyrosine kinase Zap70, the inventors tested the compound on cells obtained from a primary T-cell lymphoma (TLS10med), a primary B-cell lymphoma (tumormed) or a murine BL cell line we have shown previously to be susceptible to R406 (TBL-1). The cells were incubated for an MTS assay as described elsewhere herein. The cells tested were a cell line derived from a murine Burkitt's lymphoma tumor (TBL-1), a cell line derived from a T-cell lymphoma tumor (TLS10) or cells derived from the spleen and lymph nodes of a primary tumor bearing Eμ-MYC/BCR^{HEL}/sHEL mouse (pooled at a 1:1 ratio).

In order to specifically determine whether the R406 compound is inhibiting cell proliferation, survival, or both, and in what order, tumor cells are labeled with CFSE and exposed to the drug. Those cells are then costained with 7AAD prior to analysis. These experiments will determine whether the R406 compound is affecting cell proliferation, survival, or both, on the BL tumor cells.

### Example 7

The following example describes a general scheme for the testing of drug candidates in vivo, using mouse models of NHL of the invention, via targeting of the exemplary target, Syk.

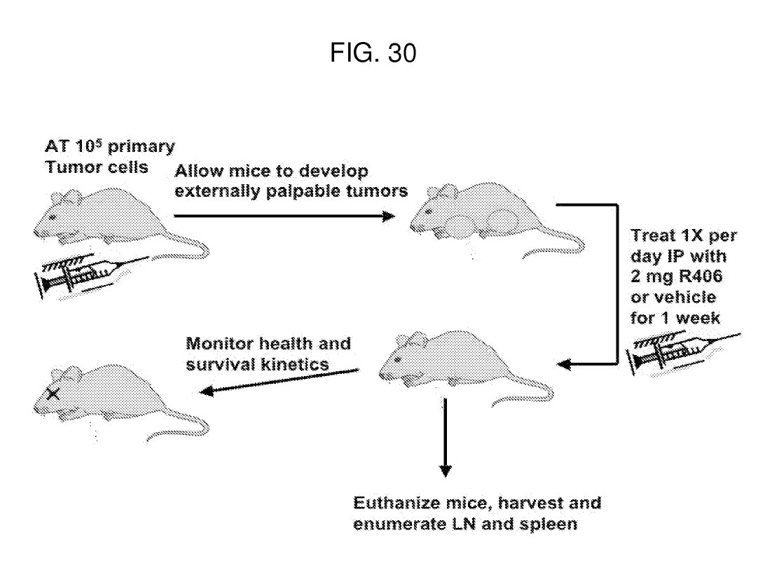

A schematic protocol for testing of drug candidates in vivo, using mouse models of NHL of the invention, is shown in FIG. 30. While the drawing illustrates the testing of the Syk inhibitor, R406, any drug candidate can be substituted into this general protocol. Cohorts of age and sex matched, syngeneic mice (C57/BL6) are given transplants of tumor cells administered by tail vein injection (inoculum ranges from 10²-10⁶ cells, but all of the mice in one cohort usually get the same dose of cells). The mice are observed daily for 14-21 days for presentation of externally evident clinical signs of hematological malignancies. These include splenomegaly and lymphadenopathy, dehydration, scruffy fur, hunched posture, labored breathing, and an ascending hind limb paralysis. When the mice are visibly sick, they begin treatment with the drug candidate. The mice are either analyzed 24 hours after the last dose of drug in order to ascertain the degree of remission caused by the drug, in an acute manner, or left in the vivarium to determine the extent of survival that is afforded by the treatment, over the normal rate of mortality for untreated, tumor-bearing mice.

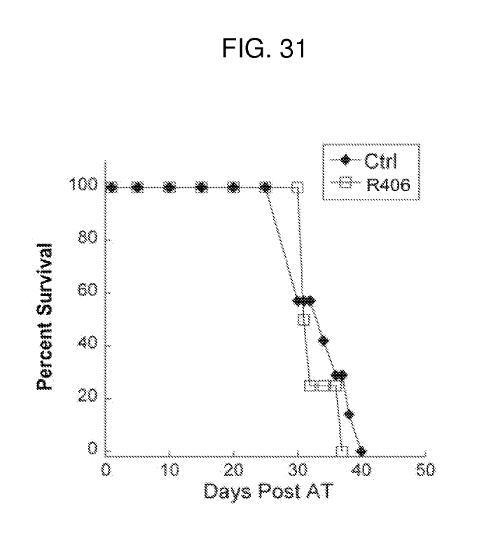

When the Syk inhibitor, R406, was tested in this model, the results showed that pharmacological inhibition of Syk in B-cell lymphomas that do not express a B cell receptor on the surface (resemble pre-B cell Acute lymphocytic leukemia/lymphoma of humans) does not extend the survival of tumor-bearing mice (FIG. 31). In this experiment, cohorts of 10 mice each were given transplants of tumors derived from Eμ-MYC mice. 21 days later, when the mice presented externally evident signs of disease, they were treated for 7 days with R788 (a prodrug called R788 that after being metabolized in the liver yields a compound substantially similar to R406). The mice were then observed for survival, and perished at the same rate and time frame as their untreated, tumor-bearing counterparts.

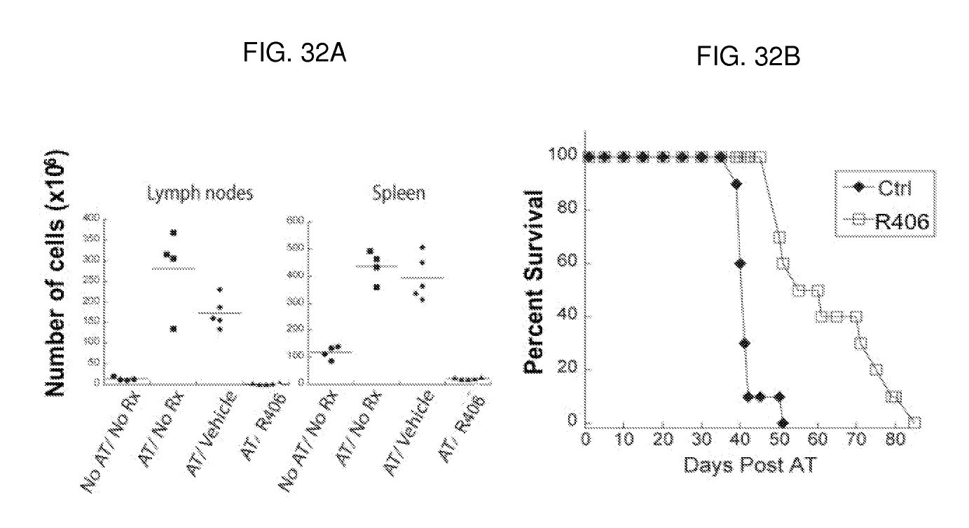

The results shown in FIG. 32 show that pharmacological inhibition of Syk in mice harboring murine B-CLL tumors caused significant remission and extended lifespan of treated tumor-bearing mice. In this experiment, cohorts of 20 mice each were given transplants of tumors derived from Eμ-MYC/BCR^{HEL} (resemble human B-CLL) mice. 24 days later, when the mice presented externally evident signs of disease, they were treated for 7 days with R788. Part of the cohort was euthanized and evaluated for remission acutely (left set of graphs). The transient treatment of B-CLL tumor-bearing mice with R788 significantly decreased the tumor burden in those mice, as determined by counting the number of cells present in the lymph nodes and spleens. In addition, the other half of the cohort was observed over time for kinetics and time frame of mortality following transient treatment with R788. The treated mice survived twice as long as their untreated counterparts (right-side graph).

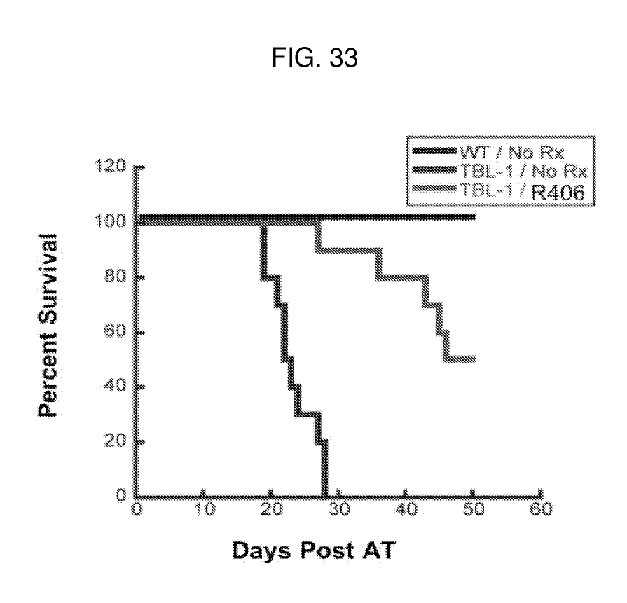

In another experiment, depicted in FIG. 33, cohorts of 10 mice each were given transplants of tumors derived from Eμ-MYC/BCR^{HEL}/sHEL (resemble human BL) mice. 20 days later, when the mice presented externally evident signs of disease, they were treated for 7 days with R788. This cohort was observed over time for kinetics and time frame of mortality following transient treatment with R788. The treated mice survived at least twice as long as their untreated counterparts (right-side graph), showing that pharmacological inhibition of Syk in mice harboring murine BL tumors caused significant remission and extended lifespan of treated tumor-bearing mice.

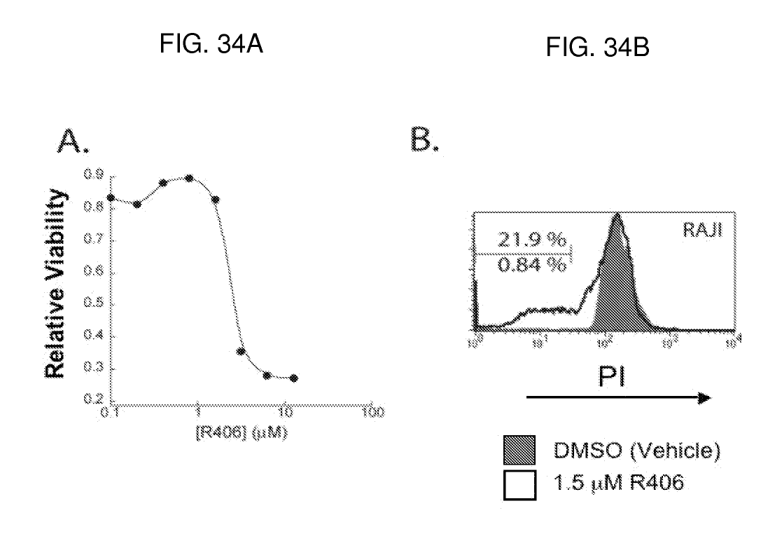

In order to validate the observations obtained in the mouse models of BL, the inventors tested whether the pharmacological inhibition of Syk in a human BL cell line (Raji) also results in the reduced viability of those cells. FIG. 34A shows the dose response decrease in viability of Raji cells to increasing concentrations of R406, as determined with an MTS assay. FIG. 34B shows that the cells undergo apoptosis following exposure to R406 in vitro, as determined by observing the appearance of a subdiploid population in a propidium iodide stain. Similar results are observed upon the shRNA-mediated disruption of Syk expression in Raji cells. Accordingly, these results demonstrate that human BL cell lines undergo apoptosis in vitro following pharmacological inhibition of Syk.

### Example 8

The following example shows that the NHL mouse models described herein can be used to generate cell lines that can be used for high-throughput screening of drug candidates.

Each of the mouse models described herein have yielded cell lines that can be used for high-throughput screening of drug candidates for further development in treatment of tumor bearing individuals. To perform such a screening, cell lines are obtained from primary lymphomas from any one or more of the NHL mouse models of the invention, or one can use cell lines derived from any of the mouse models of disease described herein. For the viability assays, 5×10⁴ cells are cultured in the presence of one of 10 different concentrations of a drug, for 12 hours. After the 12 hour incubation, the wells are pulsed with 10 μl of MTS reagent (Promega) and cultured for another two hours. All plates are read with an ELISA plate reader (O.D. 490). Cultures are set up in a final volume of 200 μl, in 96-well plates. All experimental conditions are set up in duplicate wells, in three independent experiments.

The drug screen demonstrates that the NHL mouse models of the invention, and cell lines derived therefrom, are powerful tools for use in high-throughput screening of drug candidates and can identify both useful drugs and novel targets related to NHL.

Each reference or publication cited herein is incorporated herein by reference in its entirety for all purposes, including U.S. Provisional Application No. 60/820,478.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following claims.

1. A non-human animal model of B cell chronic lymphocytic leukemia/lymphoma (B-CLL), wherein the animal model is a transgenic non-human animal that expresses the following transgenes:
a) a MYC transgene that is overexpressed in the B cell lineage in the animal; and
b) a transgene encoding a pre-rearranged B cell receptor (BCR) that is overexpressed in the B cell lineage of the animal.

2. A non-human animal model of B cell chronic lymphocytic leukemia/lymphoma (B-CLL), wherein the animal model is a transgenic non-human animal that expresses the following transgenes:
a) a MYC transgene that is overexpressed in the B cell lineage in the animal;
b) a transgene encoding a non-rearranged Ig heavy chain of a B cell receptor (BCR) that selectively binds to an antigen;
c) a transgene encoding a lambda light chain of a BCR that binds to the antigen of (b).

3. A non-human animal model of Burkitt's Lymphoma (BL), wherein the animal model is a transgenic non-human animal that expresses the following transgenes:
a) a MYC transgene, wherein the MYC transgene comprises a nucleic acid sequence encoding MYC coupled to an inducible expression control sequence, wherein B cell-specific expression of the MYC transgene can be selectively repressed;
b) a transgene encoding a pre-rearranged B cell receptor (BCR) that is overexpressed in the B cell lineage of the animal and that selectively binds to an antigen; and
c) a transgene encoding a soluble form of the antigen bound by the BCR in (ii), wherein the transgene is expressed so that it is available systemically in the animal; wherein B cell-specific expression of the MYC transgene is not repressed in the animal.

4. A non-human animal model of Burkitt's Lymphoma (BL), wherein the animal model is a transgenic non-human animal that expresses the following transgenes:
a) a MYC transgene that is overexpressed in the B cell lineage in the animal;
b) a pre-rearranged VDJ region of an Ig heavy chain of a B cell receptor (BCR) that selectively binds to an antigen, wherein the transgene is designed to be integrated into the Ig heavy chain locus of the animal;
c) a transgene encoding a lambda light chain of a BCR that binds to the antigen of (ii); and
d) a transgene encoding a soluble form of the antigen in (ii) and (iii), wherein the transgene is expressed so that it is available systemically in the animal.

5. A non-human animal model of Burkitt's Lymphoma (BL), wherein the animal model is a transgenic non-human animal that expresses the following transgenes:
a) a MYC transgene that is overexpressed in the B cell lineage in the animal; and
b) a transgene encoding a pre-rearranged B cell receptor (BCR) that is overexpressed in the B cell lineage of the animal and that binds to an endogenous self-antigen expressed by the animal.

6. A non-human animal model of Follicular Like Lymphoma (FLL), comprising a transgenic non-human animal that expresses:
a) a MYC transgene, wherein the MYC transgene comprises a nucleic acid sequence encoding MYC coupled to an inducible expression control sequence, wherein B cell-specific expression of the MYC transgene can be selectively repressed;
b) a transgene encoding a pre-rearranged B cell receptor (BCR) that is overexpressed in the B cell lineage of the animal and that selectively binds to an antigen; and
c) a transgene encoding a soluble form of the antigen bound by the BCR in (b), wherein the transgene is expressed so that it is available systemically in the animal;
wherein B cell-specific expression of the MYC transgene was repressed in the animal from the birth of the animal until the animal was an adult, followed by a lowered level of continued repression of the expression of the MYC transgene, to induce FLL in the animal.

7. A non-human animal model of Follicular Like Lymphoma (FLL), comprising a transgenic non-human animal that expresses:
a) a MYC transgene, wherein the MYC transgene comprises a nucleic acid sequence encoding MYC coupled to an inducible expression control sequence, wherein B cell-specific expression of the MYC transgene can be selectively repressed;
b) a transgene encoding a pre-rearranged B cell receptor (BCR) that is overexpressed in the B cell lineage of the animal and that binds to an endogenous self-antigen expressed by the animal;
wherein B cell-specific expression of the MYC transgene was repressed in the animal from the birth of the animal until the animal was an adult, followed by a lowered level of continued repression of the expression of the MYC transgene, to induce FLL in the animal.

8. A non-human animal model of Diffuse Large B Cell Lymphoma (DLBCL), comprising a transgenic non-human animal that expresses:
a) a MYC transgene, wherein the MYC transgene comprises a nucleic acid sequence encoding MYC coupled to an inducible expression control sequence, wherein B cell-specific expression of the MYC transgene can be selectively repressed;
b) a transgene encoding a pre-rearranged B cell receptor (BCR) that is overexpressed in the B cell lineage of the animal and that selectively binds to an antigen; and
c) a transgene encoding a soluble form of the antigen bound by the BCR in (b), wherein the transgene is expressed so that it is available systemically in the animal;
wherein the B cell-specific expression of the MYC transgene in the animal was repressed from the birth of the animal until the animal was an adult, followed by cessation of the repression of the expression of the MYC transgene in the animal, to induce DLBCL in the animal.

9. A non-human animal model of Diffuse Large B Cell Lymphoma (DLBCL), comprising a transgenic non-human animal that expresses:
a) a MYC transgene, wherein the MYC transgene comprises a nucleic acid sequence encoding MYC coupled to an inducible expression control sequence, wherein B cell-specific expression of the MYC transgene can be selectively repressed;
b) a transgene encoding a pre-rearranged B cell receptor (BCR) that is overexpressed in the B cell lineage of the animal and that binds to an endogenous self-antigen expressed by the animal;
wherein the B cell-specific expression of the MYC transgene in the animal was repressed from the birth of the animal until the animal was an adult, followed by cessation of the repression of the expression of the MYC transgene in the animal, to induce DLBCL in the animal.

10. The non-human animal model of claim 1, wherein the MYC transgene comprises a nucleic acid sequence encoding MYC coupled to an Ig heavy chain enhancer.

11. The non-human animal model of claim 10, wherein the MYC transgene is Eμ-MYC.

12. The non-human animal model of claim 1, wherein the MYC transgene comprises a nucleic acid sequence encoding MYC coupled to an inducible expression control sequence, wherein B cell-specific expression of the MYC transgene can be selectively repressed.

13. The non-human animal model of claim 3, wherein the MYC transgene comprises a nucleic acid sequence encoding Myc coupled to a tetracycline (TET) responsive expression control element (TRE), the expression of which is controlled by a B-cell specific TET-off repressor.

14. The non-human animal model of claim 13, wherein the B-cell specific TET-off repressor is a mouse mammary tumor virus long term repeat (MMTV-LTR) driving expression of reverse tetracycline-dependent transactivator (rtTA) (MMTV-rtTA).

15. The non-human animal model of claim 13, wherein the MYC transgene is TRE-MYC and wherein the animal also expresses an MMTV-rtTA transgene.

16. The non-human animal model of claim 3, wherein the expression of the MYC transgene can be selectively repressed by administration of tetracycline or doxycycline.

17. The non-human animal model of claim 1, wherein the BCR transgene is expressed under the control of the endogenous immunoglobulin promoter.

18. The non-human animal model of claim 1, wherein the BCR transgene selectively binds to hen egg lysozyme (HEL).

19. The non-human animal model of claim 1, wherein the BCR transgene comprises:
a) a pre-rearranged VDJ region of an Ig heavy chain of a B cell receptor (BCR) that selectively binds to an antigen, wherein the transgene is designed to be integrated into the Ig heavy chain locus of the animal; and
b) a transgene encoding a lambda light chain of a BCR that binds to the antigen of (b).

20. The non-human animal model of claim 19, wherein the BCR transgene selectively binds to hen egg lysozyme (HEL).

21. The non-human animal model of claim 5, wherein the BCR binds to arsenate and to an endogenous self-antigen in the animal.

22. The non-human animal model of claim 21, wherein the BCR transgene is ARS.A1.

23. The non-human animal model of claim 3, wherein the transgene encoding the soluble form of the antigen is sHEL.

24. The non-human animal model of claim 1, wherein the animal is a rodent.

25. The non-human animal model of claim 1, wherein the animal is a mouse.

26. The non-human animal model of claim 1, wherein the animal model of B-CLL is a transgenic mouse that expresses the following transgenes:
a) Eμ-MYC; and
b) BCL^{HEL}.

27. The non-human animal model of claim 1, wherein the animal model of B-CLL is a transgenic mouse that expresses the following transgenes:
a) TRE-MYC;
b) MMTV-rtTA; and
c) BCL^{HEL}.

28. The non-human animal model of claim 2, wherein the animal model of B-CLL is a transgenic mouse that expresses the following transgenes:
a) Eμ-MYC;
b) VDJki; and
c) Lt-tg.

29. The non-human animal model of claim 2, wherein the animal model of B-CLL is a transgenic mouse that expresses the following transgenes:
a) TRE-MYC;
b) MMTV-rtTA;
c) VDJki; and
d) Lt-tg.

30. The non-human animal model of claim 3, wherein the animal model of BL is a transgenic mouse that expresses the following transgenes:
a) TRE-MYC;
b) MMTV-rtTA;
c) BCL^{HEL}; and
d) sHEL;
wherein B cell-specific expression of the TRE-MYC transgene is not repressed in the animal.

31. The non-human animal model of claim 4, wherein the animal model of BL is a transgenic mouse that expresses the following transgenes:
a) Eμ-MYC;
b) VDJki;
c) Lt-tg; and
d) sHEL.

32. The non-human animal model of claim 5, wherein the animal model of BL is a transgenic mouse that expresses the following transgenes:
a) Eμ-MYC; and
b) Ars.A1.

33. The non-human animal model of claim 6, wherein the animal model of FLL is a transgenic mouse that expresses the following transgenes:
a) TRE-MYC;
b) MMTV-rtTA;
c) BCL^{HEL}; and
d) sHEL;
wherein B cell-specific expression of the TRE-MYC transgene was repressed in the animal by administration of tetracycline or doxycycline from the birth of the animal until the animal was an adult, followed by continued administration of a lower amount of tetracycline or doxycycline to the animal, to induce FLL in the animal.

34. The non-human animal model of claim 7, wherein the animal model of FLL is a transgenic mouse that expresses the following transgenes:
a) TRE-MYC;
b) MMTV-rtTA; and
c) ARS.A1;
wherein B cell-specific expression of the TRE-MYC transgene was repressed in the animal by administration of tetracycline or doxycycline from the birth of the animal until the animal was an adult, followed by continued administration of a lower amount of tetracycline or doxycycline to the animal, to induce FLL in the animal.

35. The non-human animal model of claim 8, wherein the animal model of DLBCL is a transgenic mouse that expresses the following transgenes:
a) TRE-MYC;
b) MMTV-rtTA;
c) BCL^{HEL}; and
d) sHEL;
wherein the B cell-specific expression of the TRE-MYC transgene in the animal was repressed by administration of tetracycline or doxycycline from the birth of the animal until the animal was an adult, followed by cessation of the administration of tetracycline or doxycycline to the animal, to induce DLBCL in the animal.

36. The non-human animal model of claim 9, wherein the animal model of DLBCL is a transgenic mouse that expresses the following transgenes:
a) TRE-MYC;
b) MMTV-rtTA; and
c) ARS.A1;
wherein the B cell-specific expression of the TRE-MYC transgene in the animal was repressed by administration of tetracycline or doxycycline from the birth of the animal until the animal was an adult, followed by cessation of the administration of tetracycline or doxycycline to the animal, to induce DLBCL in the animal.

37. A B cell isolated from any the non-human animal model of claim 1.

38. A B cell line produced from a B cell isolated from the non-human animal model of claim 1.

39. B cell tumors isolated from the non-human animal model of claim 1.

40. A panel of transgenic mice for evaluation of non-Hodgkin's lymphomas (NHL), comprising two or more different transgenic mice selected from the group consisting of:
a) a transgenic non-human animal according to claim 1, wherein the non-human animal is a mouse; and
b) a transgenic mouse model of Burkitt's Lymphoma (BL), wherein the transgenic mouse expresses:
i) a MYC transgene that is overexpressed in the B cell lineage in the animal;
ii) a transgene encoding a pre-rearranged B cell receptor (BCR) that is overexpressed in the B cell lineage of the animal; and
iii) a transgene encoding a soluble form of the antigen bound by the BCR in (ii), wherein the transgene is expressed so that it is available systemically in the animal.

41. A panel of transgenic mice for evaluation of non-Hodgkin's lymphomas (NHL), comprising two or more different transgenic mice selected from the group consisting of:
a) a transgenic mouse of claim 26; and
b) a transgenic mouse model of BL, wherein the transgenic mouse expresses the following transgenes:
i) Eμ-MYC;
ii) BCR^{HEL}; and
iii) sHEL.

42. A panel of two or more B cell lines, comprising at least one B cell line was produced from B cells isolated from a transgenic mouse of claim 40.

43. A method for preclinical testing of drug candidates for non-Hodgkin's Lymphoma (NHL), comprising administering to the non-human animal model of claim 1 a candidate drug for NHL, and detecting whether the candidate drug inhibits tumors in the animal model, wherein candidate drugs that inhibit tumors in the animal model are selected for clinical testing.

44. A method for preclinical testing of drug candidates for non-Hodgkin's Lymphoma (NHL), comprising administering to two or more mouse models in the panel of mice of claim 40 a candidate drug for NHL, and detecting whether the candidate drug inhibits tumors in the one or more of the mouse models in the panel of mice, wherein candidate drugs that inhibit tumors in at least one mouse model is selected for clinical testing.

45. The method of claim 44, wherein a candidate drug that inhibits tumors in a first mouse model but not in a second mouse model is selected for clinical testing as a specific inhibitor of the form of NHL exhibited by the first mouse model.

46. The method of claim 43, wherein the mouse model has been further genetically modified to comprise a human nucleic acid molecule of interest or to express a human protein, wherein the nucleic acid molecule or protein is a target for human NHL, and wherein the method includes a step of detecting whether the candidate drug changes the expression or biological activity of the target as compared to in the absence of the candidate drug.

47. The method of claim 46, wherein the target is expressed by the tumors of the mouse model.

48. A method for preclinical testing of drug candidates for non-Hodgkin's Lymphoma (NHL), comprising contacting the B cell line of claim 38 with a candidate drug for NHL, and detecting whether the B cell line is sensitive to the candidate drug, wherein candidate drugs to which the B cell line is sensitive is selected for clinical testing.

49. A method for preclinical testing of drug candidates for non-Hodgkin's Lymphoma (NHL), comprising administering to two or more of the B cell lines in the panel of B cell lines of claim 42 a candidate drug for NHL, and detecting whether one or more of the B cell lines is sensitive to the candidate drug, wherein candidate drugs to which at least one B cell line is sensitive is selected for clinical testing.

50. The method of claim 49, wherein a candidate drug to which a B cell line from a first mouse model is sensitive, but to which a B cell line from a second mouse model is not sensitive, is selected for clinical testing as a specific inhibitor of the form of NHL exhibited by the first mouse model.

51. The method of claim 48, wherein the B cell line has been genetically modified to comprise a human nucleic acid molecule of interest or to express a human protein, wherein the nucleic acid molecule or protein is a target for human NHL, and wherein the method includes a step of detecting whether the candidate drug changes the expression or biological activity of the target as compared to in the absence of the candidate drug.

52. A method to identify a target for use in the diagnosis, study or treatment of a Non-Hodgkin's Lymphoma (NHL) or condition related thereto, comprising comparing the expression of genes by the animal model of claim 1 to the expression of genes by a control animal that does not have an NHL, and identifying genes that are differentially expressed in the NHL animal model, or the proteins encoded by the genes, as targets for use in the diagnosis, study or treatment of NHL.

53. A method to identify a target for use in the diagnosis, study or treatment of a Non-Hodgkin's Lymphoma (NHL) or condition related thereto, comprising comparing the expression of genes by two or more of the mouse models in the panel of mice of claim 40 to each other and to the expression of the genes by a control mouse that does not have an NHL, and identifying genes that are differentially expressed in one or more of the NHL mice models, or the proteins encoded by the genes, as targets for use in the diagnosis, study or treatment of NHL.

54. The method of claim 53, wherein a gene that is differentially expressed in a first mouse model but not in a second mouse model is selected as a specific target for the form of NHL exhibited by the first mouse model.

55. A method to identify a target for use in the diagnosis, study or treatment of a Non-Hodgkin's Lymphoma (NHL) or condition related thereto, comprising comparing the expression of genes by a B cell line of claim 38 to the expression of genes by a control B cell line from an animal that does not have NHL, and identifying genes that are differentially expressed in the B cell line from the NHL animal model, or the proteins encoded by the genes, as targets for use in the diagnosis, study or treatment of NHL.

56. A method to identify a target for use in the diagnosis, study or treatment of a Non-Hodgkin's Lymphoma (NHL) or condition related thereto, comprising comparing the expression of genes by two or more B cell lines from the panel of B cell lines of claim 42 to each other and to the expression of genes by a control B cell line from an animal that does not have NHL, and identifying genes that are differentially expressed in one or more of the B cell line from the NHL mouse models, or the proteins encoded by the genes, as targets for use in the diagnosis, study or treatment of NHL.

57. The method of claim 56, wherein a gene that is differentially expressed in B cell line from a first mouse model but not in a B cell line from a second mouse model is selected as a specific target for the form of NHL exhibited by the first mouse model.

58. A method to identify a target for use in the diagnosis, study or treatment of a Non-Hodgkin's Lymphoma (NHL) or condition related thereto, comprising comparing a biological activity of a gene or protein in the animal model of claim 1 the biological activity of the gene or protein in a control animal, wherein genes or proteins with a difference in biological activity in the animal model as compared to the control animal are selected for use in the diagnosis, study or treatment of NHL.

59. A method to identify a target for use in the diagnosis, study or treatment of a Non-Hodgkin's Lymphoma (NHL) or condition related thereto, comprising comparing a biological activity of a gene or protein two or mice in the panel of mice of claim 40 to each other and to the biological activity of the gene or protein by a control mouse that does not have an NHL, wherein genes or proteins with a difference in biological activity in one or more of the mouse models as compared to the control animal, are selected for use in the diagnosis, study or treatment of NHL.

60. The method of claim 59, wherein genes or proteins having a difference in biological activity in a first mouse model but not in a second mouse model is selected as a specific target for the form of NHL exhibited by the first mouse model.

61. A method to identify a target for use in the diagnosis, study or treatment of a Non-Hodgkin's Lymphoma (NHL) or condition related thereto, comprising comparing a biological activity of a gene or protein in a B cell line of claim 38 to the biological activity of the gene or protein in a control B cell line from an animal that does not have NHL, wherein genes or proteins with a change in biological activity in the B cell line from the animal model as compared to the B cell line from the control animal, are selected for use in the diagnosis, study or treatment of NHL.

62. A method to identify a target for use in the diagnosis, study or treatment of a Non-Hodgkin's Lymphoma (NHL) or condition related thereto, comprising comparing a biological activity of a gene or protein in two or more B cell lines from the panel of B cell lines of claim 42 to each other and to the biological activity of the gene or protein in a control B cell line from an animal that does not have NHL, wherein genes or proteins with a change in biological activity in a B cell line from the animal model as compared to the B cell line from the control animal, are selected for use in the diagnosis, study or treatment of NHL.

63. The method of claim 62, wherein a genes or protein having a difference in biological activity in a B cell line from a first mouse model but not in a B cell line from a second mouse model is selected as a specific target for the form of NHL exhibited by the first mouse model.

64. The method of claim 58, wherein the method comprises a first step of contacting the animal or cells with a test compound, prior to the step of comparing.

65. A method to inhibit a B cell Non-Hodgkin's Lymphoma (NHL), comprising inhibiting tec tyrosine kinase Syk expression or activity.

66. The method of claim 65, wherein the NHL is selected from the group consisting of: B-cell chronic lymphocytic leukemia, Burkitt's lymphoma, Follicular like lymphoma and Diffuse large B-cell lymphoma.

67. The method of claim 65, comprising administering to an NHL an shRNA that selectively binds to Syk and inhibits the expression of Syk.

68. The method of claim 65, comprising administering to an NHL a drug that inhibits the expression or activity of Syk.

69. A non-human animal model of Non-Hodgkin's Lymphoma (NHL), wherein the animal model is a transgenic non-human animal that overexpresses MYC in an autoreactive B cell background.

70. A non-human animal model of Non-Hodgkin's Lymphoma (NHL), wherein the animal model is a transgenic non-human animal that overexpresses MYC in a background where the B cell receptor is tonically or constitutively expressed.

71. The non-human animal model of claim 69, wherein the non-human animal model is a mouse.

72. An isolated non-human animal egg, wherein the egg contains the transgenes expressed by the non-human animal model in claim 1.

73. A part of a non-human animal model in claim 1, selected from the group consisting of: a cell, a tissue, an organ or a bodily fluid.
